(19) United States
(12) Patent Application Publication
(10) Pub. No.: US 2018/0162896 A1
(43) Pub. Date: Jun. 14, 2018

(54) Synthesis of Phosphoramidates

(71) Applicant: Sandoz AG, Basel (CH)

(72) Inventors: Thorsten Wilhelm, Kundl (AT); Olga Schöne, Kundl (AT); Hans-Peter Spitzenstätter, Kundl (AT)

(73) Assignee: Sandoz AG, Basel (CH)

(21) Appl. No.: 15/575,771

(22) PCT Filed: May 26, 2016
(86) PCT No.: PCT/EP2016/061814

§ 371 Date: Nov. 20, 2017

(30) Foreign Application Priority Data
May 26, 2015 (EP) 15169168.0
Feb. 3, 2016 (EP) 16154143.8

(51) Int. Cl. C07H 19/10

(52) U.S. Cl. CPC C07H 19/10

(57) ABSTRACT

A process for preparing a nucleoside phosphoramidate, in particular to a process for preparing sofosbuvir, wherein a phosphoramidate derivative is used as starting material.

The present invention relates to a process for preparing a nucleoside phosphoramidate, in particular to a process for preparing sofosbuvir, wherein a phosphoramidate derivative is used as starting material.

Sofosbuvir according to the following formula

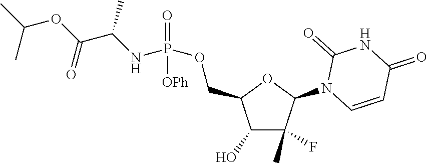 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

with IUPAC name (S)-isopropyl 2-(((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy) (phenoxy)phosphoryl) amino)propanoate is a drug inhibiting the RNA polymerase used by the hepatitis C virus to replicate its RNA.

For preparing sofosbuvir, WO 2008/121634 discloses a process wherein a nucleoside is reacted with a phosphoric acid amide having chloride as leaving group. N-methylimidazole is used in the displacement reaction. Due to the chirality of the phosphorous atom, two diastereoisomers are obtained which have the following formulas (I-1) and (1-2):

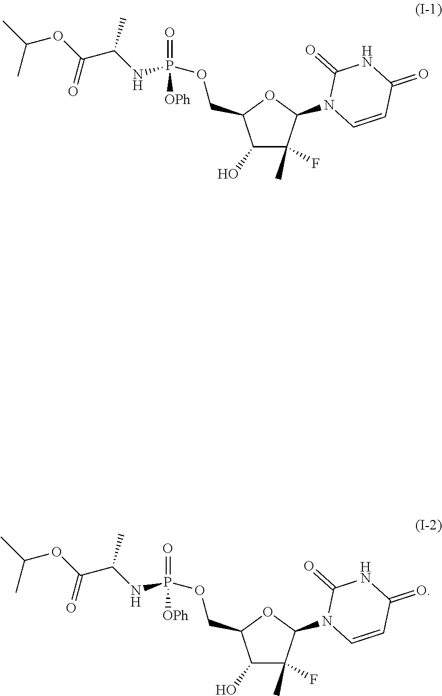 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1 ; CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

This displacement reaction taught in WO 2008/121634 A in the presence of N-methylimidazole is not selective, and a 1:1 mixture of the two diastereomers of formulas (I-1) and (1-2) is obtained. The reaction is further plagued with significant formation of the doubly phosphorylated side product (in positions 5′ and 3′), resulting in a tedious purification process and in low yield.

As an alternative to the phosphorochloridate of WO 2008/121634, WO 2010/135569 A, WO 2011/123668, WO 2012/012465 A, J. Org. Chem. 2011, 76, 8311, WO 2014/047117 A, and WO 2014/164533 A disclose crystalline phosphoramidating reagents which bear electron-poor phenolates and heterocycles as leaving groups. These reagents can be isolated in diastereomerically enriched form by fractional crystallization or chromatography. The process necessitates diastereomer separation of phosphoramidating reagents prior to the coupling reaction, which makes the process less than ideal.

Additionally, leaving groups, such as aryloxide substituted with at least one electron-withdrawing group such as a halogen or a nitro group, lead to the problem of the formation side-products such as 3′-O-phosphoramidate or 3′,5′-bis-O-phosphoramidate. In Example 15 of WO 2010/135569 A, in order to overcome the problem of the formation of 3′-O-phosphoramidate or 3′,5′-bis-O-phosphoramidate side products, it is proposed to protect position 3′ of the ribose ring with a levulinic anhydride and subsequently de-protect said position. Alternatively, position 3′ is protected with a tert-butyl-dimethylsilyl group. Therefore, comparatively complicated methods have to be applied to overcome said problems. WO 2011/123672 A describes a process for preparing nucleoside phosphoramidate compounds wherein the nucleoside phosphoramidate is prepared via displacement of the leaving group on a phosphoramidate to give the corresponding nucleoside-phosphoramidate. The leaving group is either aryloxide substituted with at least one electron-withdrawing group such as halogen or a nitro group or benzo[d]thiazole-2(3H)-thione. WO 2014/047117 A discloses a process for preparing nucleoside phosphoramidate compounds, in particular a complicated two-step process. The first step is the displacement of the leaving group such as p-nitrophenol on a phosphinoborane derivative or on a thio-phosphoramidate compound to give the corresponding nucleoside boran- or thio-phosphoramidate. The displacement occurs in basic conditions (Et₃N, DBU (1,8-Diazabicyclo[5.4.0]undec-7-ene)). In a subsequent step, the nucleoside boran- or thio-phosphoramidate is oxidized to the corresponding nucleoside phosphoramidate. In addition to the problems regarding comparatively complicated process, there is the problem relating to the chemical nature of the leaving groups on the phosphoramidate. These leaving groups remain as trace impurity in the final nucleoside phosphoramidate compounds. However, many leaving groups such as p-nitrophenol or other aryloxide groups substituted with an electron-withdrawing group are considered to be toxic substances, in particular genotoxic substances, by FDA. Generally, difficulties may be encountered to purify the final API from these toxic leaving groups to meet the FDA requirements.

Therefore, the problem underlying the present invention is the provision of a novel process for preparing nucleoside phosphoramidates, in particular sofosbuvir, that starts from a phosphoramidate having the toxicologically harmless succinimide as leaving group, wherein the process exhibits an improved diastereoselectivity to the valuable product, in particular sofosbuvir. The diastereoselectivity achieved with the known process taught in the prior art making use of the phosphoramidates having chloride as leaving group and using N-methylimidazole (NMI) as the base is lower than the diastereoselectivity achieved with the process of the invention.

Further, it was surprisingly found that the problem of providing a diastereoselective process can be solved by a process for preparing nucleoside phosphoramidates, in particular sofosbuvir, which is carried out using a base preferably an organic base, more preferably an organic nitrogenous base in combination with a Lewis acid. In particular, the present inventors have surprisingly found that slightly enriched or completely P-racemic mixtures of the phosphoramidate derivative according to the present invention gave nucleoside phosphoramidate with high diastereoselectivity when reacted in the presence of a Lewis acid and a base according to the invention. Without being bound to any theory, it is believed that the high diastereoselectivity is because the phosphoramidating reagent undergoes dynamic kinetic resolution during the coupling reaction. In this case both diastereoisomers of the phosphoramidating reagent exist in equilibrium and only one goes on to form the desired diastereoisomer of sofosbuvir.

Hence, the process of the present invention advantageously avoids waste of material (such as non-useful diastereoisomers) and translates directly into a faster and more economical process.

It has further been found that the process according to the present invention leads to high yields.

### PROCESS ACCORDING TO THE INVENTION CARRIED OUT IN THE PRESENCE OF A BASE AND A LEWIS ACID

Therefore, the present invention relates to a process for preparing of a compound of formula (I)

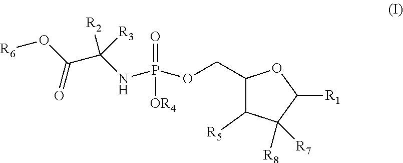

or a salt thereof, the process comprising
a) reacting a compound of formula (II)

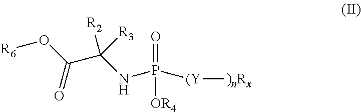

with a compound of formula (III)

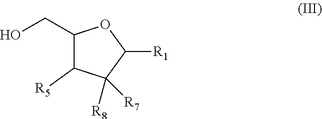

in the presence of a Lewis acid and a base, preferably an organic base according to the present invention, obtaining a mixture comprising the compound of formula (I).

Therefore, the present invention relates to a process for preparing of a compound of formula (I)

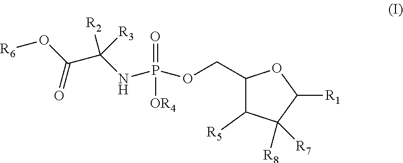

or a salt thereof, the process comprising
a) reacting a compound of formula (II)

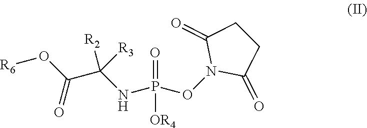

with a compound of formula (III)

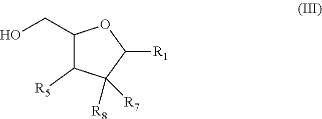

in the presence of a Lewis acid and a base, preferably an organic base according to the present invention, obtaining a mixture comprising the compound of formula (I).

Preferably (Y—)ₙRₓ is a leaving group for nucleophilic substitution reaction, wherein n is 0 or 1 and wherein Y is O, N or S.

Preferably, Rₓ is alkyl, aryl, or heteroaryl, each optionally substituted with one or more electron-withdrawing groups, preferably aryl optionally substituted with one or more electron-withdrawing groups, more preferably phenyl optionally substituted with one or more electron-withdrawing groups, more preferably phenyl substituted with one or more electron-withdrawing groups, wherein the one or more electron-withdrawing groups are preferably F, Cl, Br, I, or NO₂; or

when n is 1, R_{R} is
a residue of formula (A)

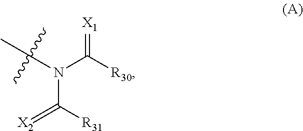

a residue of formula (B)

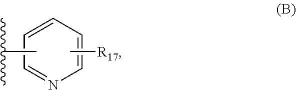

a residue of formula (C)

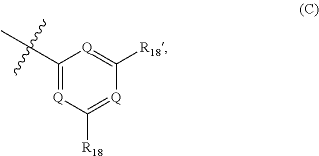

or a residue of formula (D)

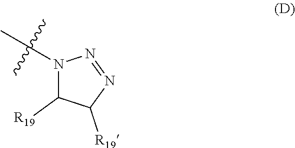

or when n is 0,
Rₓ is a residue of formula (A1)

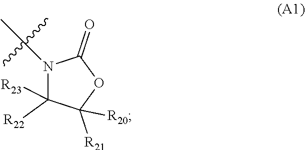

wherein at each occurrence
X₁ and X₂ are independently O or S;
R₃₀ and R₃₁ are independently H, OH, NH₂, C₁-C₆ alkyl or C₁-C₆ alkoxy, or R₃₀ and R₃₁, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group, preferably F, Cl, Br, I, NO₂, CHO, COOH, COO—(C₁-C₆)alkyl, CN, or COCl;
R₁₈ and R₁₈, are independently F, Cl, Br, I, or C₁-C₆ alkoxy;
each Q is independently C or N, wherein at least one Q is N;
R₁₉ and R₁₉, are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl;
R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.

It is further conceivable that when n is 0, Rₓ can also be Cl. In this case in the process according to the invention, it is preferred that the base is not NMI.

More preferably, when n is 1, Rₓ is a residue of formula (A), a residue of formula (B), a residue of formula (C), or a residue of formula (D), or when n is 0, Rₓ is a residue of formula (A1).

More preferably, when n is 0, Rₓ is a residue of formula (A1)

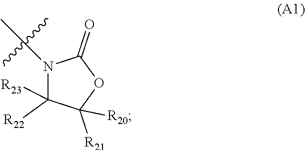

wherein R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.

According to the invention, at each occurrence, the substituent of the optionally substituted 5-, 6-, or 7-membered saturated or partially unsaturated or aromatic ring which is preferably an aryl, preferably benzo, or a heterocycle comprising one or more heteroatoms independently being N, O or S, is at least a substituent, preferably one substituent, selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl. According to the invention, at each occurrence the aromatic ring is a benzo substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.

Preferably, R₂₂ and R₂₃ are each independently H, aryl, or C₁-C₆ alkyl substituted with at least one of C₁-C₆ alkoxy optionally substituted with at least one of OH and NH₂.

Preferably, when n is 1, Rₓ is a residue of formula (A)

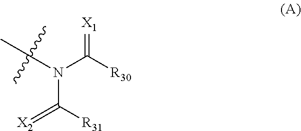

wherein
X₁ and X₂ are independently O or S;
R₃₀ and R₃₁ are independently H, OH, NH₂, C₁-C₆ alkyl or C₁-C₆ alkoxy, or R₃₀ and R₃₁, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S.

More preferably, Rₓ is a residue of formula (IIb)

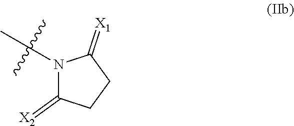

wherein X₁ is 0 and X₂ is O.

More preferably, Rₓ is a residue of formula (IIc)

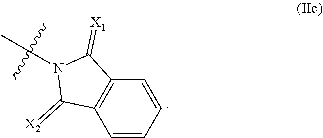

wherein X₁ is O and X₂ is O.

More preferably when n is 1 Rₓ is a residue of formula (B)

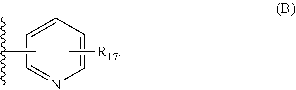

wherein R₁₇ is preferably selected from the group consisting of F, Cl, Br, I, NO₂, CHO, COOH, COO—(C₁-C₆)alkyl, CN and COCl.

More preferably when n is 1, Rₓ is a residue of formula (C)

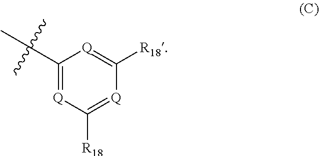

wherein R₁₈ and R₁₈, are preferably independently F, Cl, Br, I, or C₁-C₆ alkoxy and each Q is independently C or N, wherein at least one Q is N.

More preferably, when n is 1, Rₓ is a residue of formula (D)

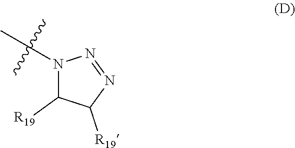

wherein R₁₉ and R_{19′} are preferably independently H, OH, NH₂, C₁-C₆ alkyl optionally substituted with at least one of OH and NH₂, or C₁-C₆ alkoxy optionally substituted with at least one of OH and NH₂; or R₁₉ and R_{19′} taken together form an optionally substituted 5-, 6-, or 7-membered saturated or partially unsaturated or aromatic ring, wherein the aromatic ring is preferably benzo, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl.

More preferably, n=1, Y═O and R_{R} is a residue of formula (IIb)

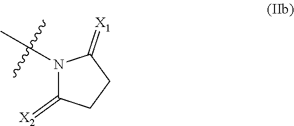

wherein X₁ and X₂ are both O.

Preferably, the residue R₄ is phenyl, naphthyl, quinolinyl, isoquinolinyl, quinazolinyl or quinoxalinyl, each optionally substituted with at least one of C₁-C₆ alkyl, C₁-C₆ alkoxy, C₃-C₆ cycloalkyl, aryl, halogen, C(O)OH, CHO, C(O)(C₁-C₆ alkyl), C(O)(aryl), C(O)O(C₁-C₆ alkyl), C(O)ONH₂, C(O)ONH(C₁-C₆ alkyl) and CN. The term “C₁-C₆ alkyl” as used herein refers to alkyl residues having 1, 2, 3, 4, 5, or 6 carbon atoms. The term “C₁-C₆ alkoxy” as used herein refers to alkoxy residues having 1, 2, 3, 4, 5, or 6 carbon atoms. The term “C₃-C₆ cycloalkyl” as used herein refers to cycloalkyl residues wherein 3, 4, 5, or 6 carbon atoms constitute the ring structure.

Preferably, the residues R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, NO₂, C(O)OH, CHO, C(O)(C₁-C₆ alkyl), C(O)(aryl), C(O)O(C₁-C₆ alkyl), C(O)ONH₂, C(O)ONH(C₁-C₆ alkyl) and CN.

Preferably, the residue R₆ is C₁-C₆ alkyl or C₃-C₁₀ cycloalkyl optionally substituted with at least one of C₁-C₆ alkyl and aryl.

Preferably, R₁ is an optionally derivatized purinyl residue, including an adenine residue and a guanine residue, or an optionally derivatized pyrimidinyl residue, including a cytosine residue, a thymine residue and an uracil residue, linked to the furanose ring according to formula (III) through a carbon or nitrogen atom.

Preferably, R₇ and R₈ are independently H, OH, F, Cl, Br, I, azide, nitrile, NH₂, NHR₂₆, NR₂₆R₂₄, C(O)NH₂, C(O)NHR₂₆, C(O)NR₂₆R₂₄, C₁-C₆ alkyl optionally substituted with C₁-C₆ alkyl, or C₃-C₁₀ cycloalkyl optionally substituted with C₁-C₆ alkyl, wherein R₂₆ and R₂₄ are independently C₁-C₆ alkyl.

Preferably, R₅ is H, OH, C₁-C₆ alkoxy, OC(O)R₂₅, or C₁-C₆ alkyl optionally substituted with C₁-C₆ alkyl or aryl, wherein R₂₅ is C₁-C₆ alkyl or aryl.

Preferably, the compound of formula (II) is

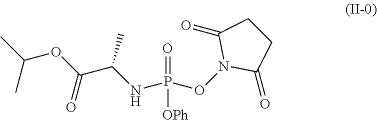 [SMILES: CC(C)OC(=O)[C@H](C)NP(=O)(Oc1ccccc1)ON1C(=O)CCC1=O]

and the compound of formula (III) is

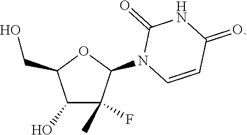 [SMILES: C[C@@]1(F)[C@H](O)[C@@H](CO)O[C@H]1n1ccc(=O)[nH]c1=O]

### Step a)

The P atom is a chirality center of the compound of formula (II) and (II-0). It is preferred that according to a), the compound of formula (II) comprises a compound of formula (II-A),

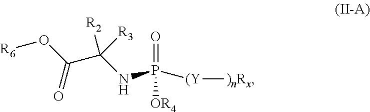

and a compound of formula (II-B),

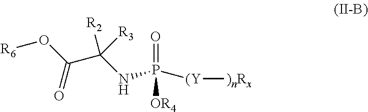

wherein the molar ratio of the compound of formula (II-A) relative the compound of formula (II-B) is preferably in the range of from 45:55 to 72:28, more preferably in the range of from 45:55 to 60:40, more preferably in the range of from 45:55 to 55:45. Preferably, the compound of formula (II) consists of the compound of formula (II-A) and the compound of formula (II-B).

Also preferably, the molar ratio of the compound of formula (II-A) relative the compound of formula (II-B) is in the range of from 55:45 to 45:55, more preferably in the range of from 54:46 to 46:54, more preferably in the range of from 53:47 to 47:53, more preferably in the range of from 52:48 to 48:52, more preferably in the range of from 51:49 to 49:51. More preferably, the molar ratio of the compound of formula (II-A) relative the compound of formula (II-B) is 1:1. Preferably, the compound of formula (II) consists of the compound of formula (II-A) and the compound of formula (II-B).

It is preferred that according to a), the compound of formula (II) comprises a compound of formula (II-a)

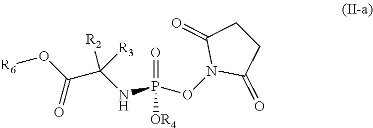

and a compound of formula (II-b)

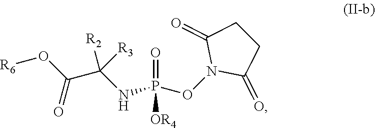

wherein the molar ratio of the compound of formula (II-a) relative the compound of formula (II-b) is in the range of from 45:55 to 72:28, preferably in the range of from 45:55 to 60: 40, more preferably in the range of from 45:55 to 55:45. More preferably, the compound of formula (II) consists of the compound of formula (II-a) and the compound of formula (II-b).

Also preferably, the molar ratio of the compound of formula (II-a) relative the compound of formula (II-b) is in the range of from 55:45 to 45:55, preferably in the range of from 54:46 to 46:54, more preferably in the range of from 53:47 to 47:53, more preferably in the range of from 52:48 to 48:52, more preferably in the range of from 51:49 to 49:51. More preferably, the molar ratio of the compound of formula (II-a) relative the compound of formula (II-b) is 1:1. More preferably, the compound of formula (II) consists of the compound of formula (II-a) and the compound of formula (II-b).

Preferably, from step a) of the process of the present invention, a mixture is obtained which comprises the compound of formula (I) wherein the compound of formula (I) comprises a compound of formula (I-1)

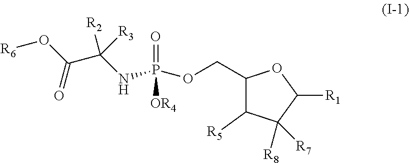

and a compound of formula (I-2)

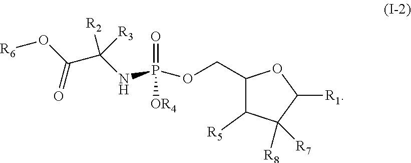

It is preferred that in the mixture obtained from a), the molar ratio of the compound of formula (I-1) relative to the compound of formula (I-2) is at least 65:35, preferably at least 70:30, more preferably at least 75:25, more preferably at least 80:20. It is conceivable that the molar ratio of the compound of formula (I-1) relative to the compound of formula (I-2) is at least 85:15 or at least 90:10 or at least 95:5 or at least 99:1 or at least 99.8:0.2. More preferably, the compound of formula (I) comprised in the mixture obtained from a) consists of the compound of formula (I-1) and the compound of formula (I-2).

It is conceivable that the molar ratio of the compound of formula (I-1) relative to the compound of formula (I-2) obtained in step a) is in the range of from 65:35 to 90:10, preferably in the range of from 75:25 to 90:10. It is conceivable that this molar ratio is further improved by crystallization or crystallization and recrystallization steps according to the present invention up to a molar ratio of the compound of formula (I-1) relative to the compound of formula (I-2) in the range of from 95:5 to 99.8:0.2, preferably in the range of from 99:1 to 99.8:0.2.

Preferably, one of R₇ and R₈ may be C₁-C₆ alkyl, preferably methyl, and one of R₇ and R₈ may be F, Cl, OH, CN, or NH₂. Further preferably, R₁ may be an optionally derivatized pyrimidinyl residue, preferably a uracil residue. Also preferably, R₁ may be an optionally derivatized purinyl residue. Therefore, among others, the following compounds of formula (I) may be preferably prepared by the process of the present invention:

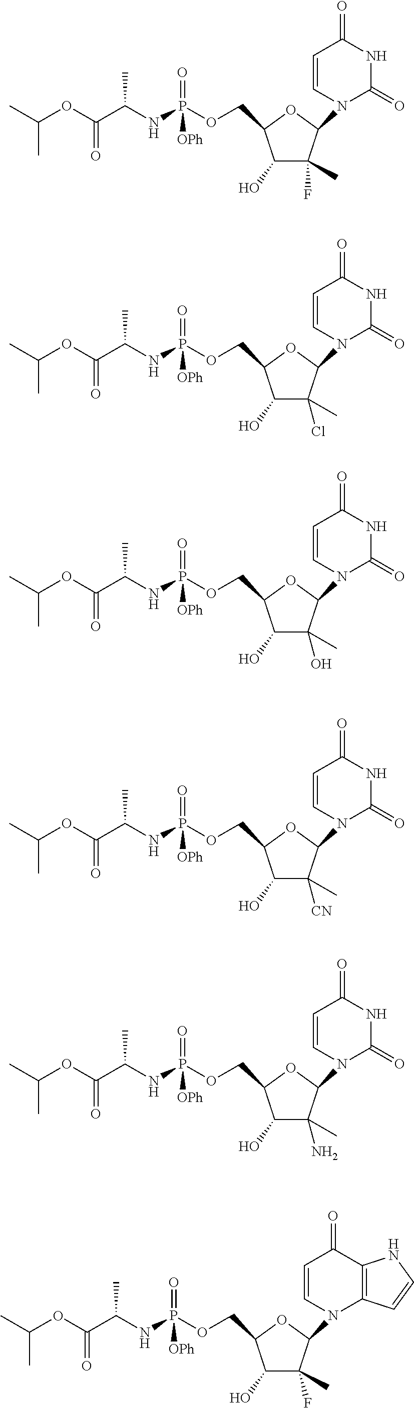

Preferably, regarding the compound of formula (III) employed in a), the residues R₇ and R₈ are independently selected from H, F, methyl or OH, more preferably the residues R₇ and R₈ are independently selected from F and methyl.

Preferably, the compound of formula (III) is

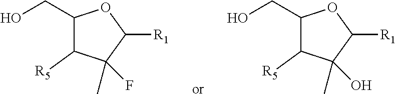

more preferably

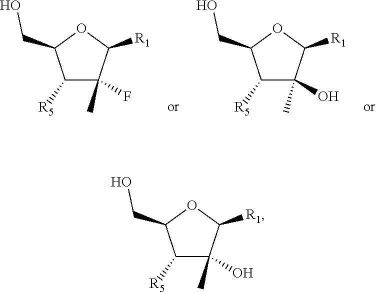

more preferably

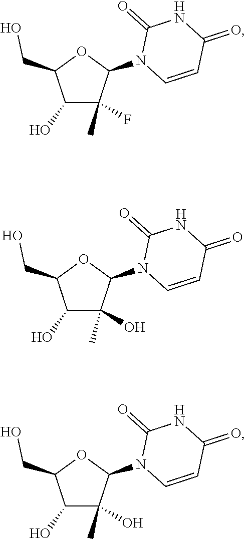 [SMILES: C[C@@]1(F)[C@H](O)[C@@H](CO)O[C@H]1n1ccc(=O)[nH]c1=O ; C[C@]1(O)[C@H](O)[C@@H](CO)O[C@H]1n1ccc(=O)[nH]c1=O ; C[C@@]1(O)[C@H](O)[C@@H](CO)O[C@H]1n1ccc(=O)[nH]c1=O]

more preferably

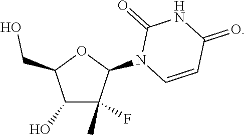 [SMILES: C[C@@]1(F)[C@H](O)[C@@H](CO)O[C@H]1n1ccc(=O)[nH]c1=O]

Preferably, the compound of formula (II-a) is a compound of formula

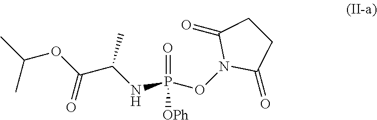 [SMILES: CC(C)OC(=O)[C@H](C)N[P@](=O)(Oc1ccccc1)ON1C(=O)CCC1=O]

and the compound of formula (II-b) is a compound of formula

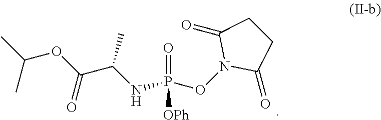 [SMILES: CC(C)OC(=O)[C@H](C)N[P@@](=O)(Oc1ccccc1)ON1C(=O)CCC1=O]

Therefore, it is preferred that the compound of formula (I-1) is one of the compounds

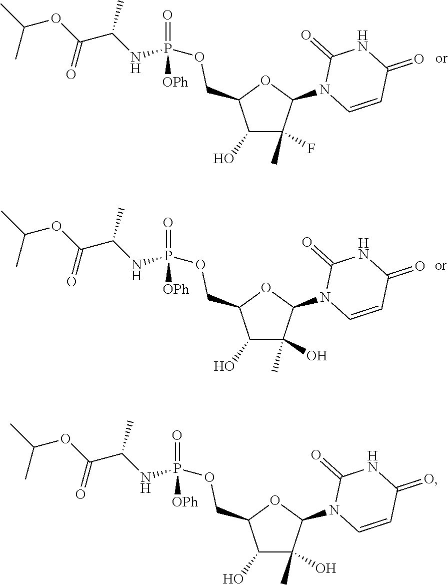 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1 ; CC(C)OC(=O)[C@H](C)N[P@](=O)(OC[C@H]1O[C@@H](n2ccc(=O)[nH]c2=O)[C@@](C)(O)[C@@H]1O)Oc1ccccc1 ; CC(C)OC(=O)[C@H](C)N[P@](=O)(OC[C@H]1O[C@@H](n2ccc(=O)[nH]c2=O)[C@](C)(O)[C@@H]1O)Oc1ccccc1]

more preferably

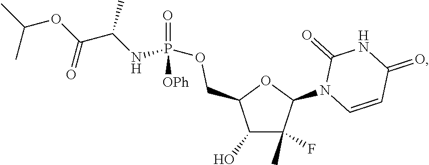 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and the compound of formula (I-2) is one of the compounds

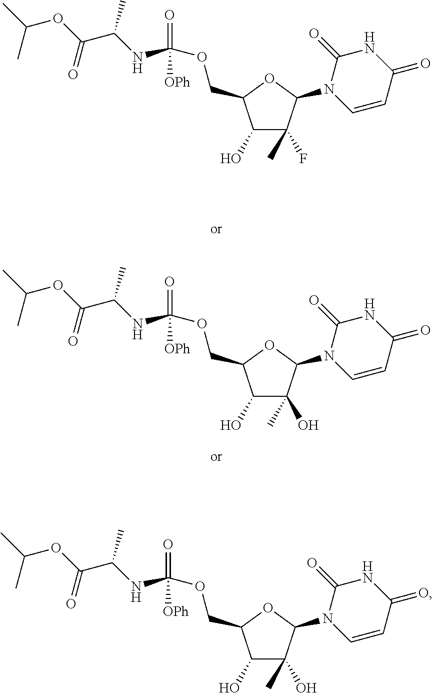

more preferably

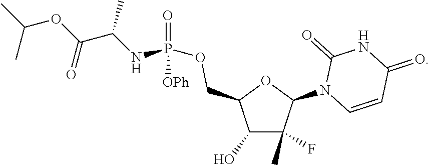 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

More preferably, the compound of formula (III) employed in a) is a compound of formula

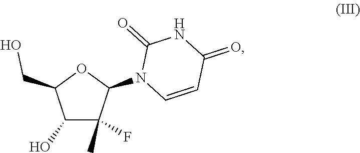 [SMILES: C[C@@]1(F)[C@H](O)[C@@H](CO)O[C@H]1n1ccc(=O)[nH]c1=O]

the compound of formula (II-0) is a compound of formula

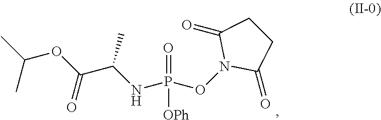 [SMILES: CC(C)OC(=O)[C@H](C)NP(=O)(Oc1ccccc1)ON1C(=O)CCC1=O]

and the compound of formula (I) is a compound of formula

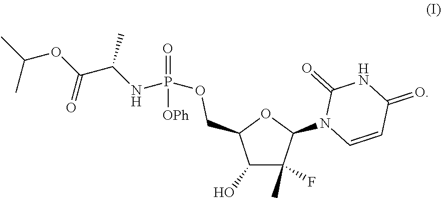 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

Therefore, the present invention also relates to a process for preparing a compound of formula (I)

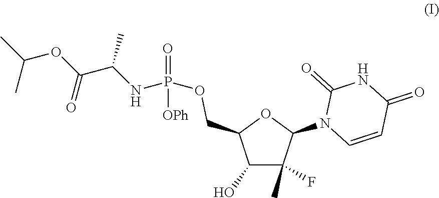 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a) reacting a compound of formula (II-0)

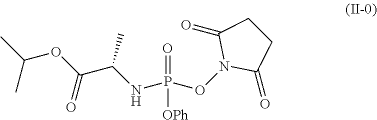 [SMILES: CC(C)OC(=O)[C@H](C)NP(=O)(Oc1ccccc1)ON1C(=O)CCC1=O]

with a compound of formula (III)

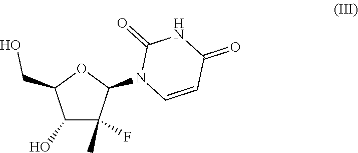 [SMILES: C[C@@]1(F)[C@H](O)[C@@H](CO)O[C@H]1n1ccc(=O)[nH]c1=O]

in the presence of a base and a Lewis acid, obtaining a mixture comprising the compound of formula (I),

wherein the compound of formula (II-0) comprises a compound of formula (II-a)

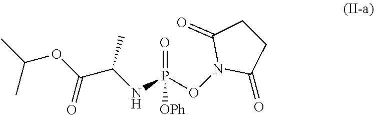 [SMILES: CC(C)OC(=O)[C@H](C)N[P@](=O)(Oc1ccccc1)ON1C(=O)CCC1=O]

and a compound of formula (II-b)

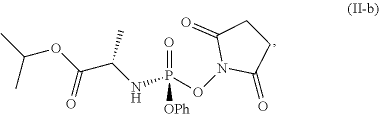 [SMILES: CC(C)OC(=O)[C@H](C)N[P@@](=O)(Oc1ccccc1)ON1C(=O)CCC1=O]

wherein the molar ratio of the compound of formula (II-a) relative the compound of formula (II-b) is in the range of from 45:55 to 72:28, preferably in the range of from 45:55 to 60:40, more preferably in the range of from 45:55 to 55:45.

Also preferably, the molar ratio of the compound of formula (II-a) relative the compound of formula (II-b) is in the range of from 55:45 to 45:55, more preferably in the range of from 54:46 to 46:54, more preferably in the range of from 53:47 to 47:53, more preferably in the range of from 52:48 to 48:52, more preferably in the range of from 51:49 to 49:51. More preferably, the molar ratio of the compound of formula (II-a) relative the compound of formula (II-b) is 1:1. More preferably, the compound of formula (II-0) consists of the compound of formula (II-a) and the compound of formula (II-b).

The base employed in a) is preferably an organic base, more preferably an organic nitrogenous base, more preferably a tertiary organic nitrogenous base. More preferably, the organic base comprises one or more of an amine, an amidine, and a heteroaromatic compound comprising a basic ring-nitrogen atom. More preferably, the organic base comprises, preferably consists of, one or more of ethyldiisopropylamine, triethylamine, diethylamine, 1,8-diazabicycloundec-7-ene, pyridine, quinoline, isoquinoline, acridine, pyrazine, imidazole, benzimidazole, ephedrine, piperidine, tetramethylguanidine, pyrazole. More preferably, the organic base comprises, preferably consists of, one or more of ethyldiisopropylamine, triethylamine, diethylamine, 1,8-diazabicycloundec-7-ene, pyridine, ephedrine, piperidine, tetramethylguanidine. More preferably, the base comprises, preferably consists of, one or more of ethyldiisopropylamine, triethylamine, 1,8-diazabicycloundec-7-ene, ephedrine, piperidine, tetramethylguanidine. More preferably, the base comprises, preferably consists, of ethyldiisopropylamine.

Therefore, the present invention preferably relates to a process for preparing a compound of formula (I)

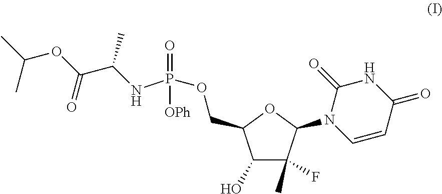 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a) reacting a compound of formula (II-0)

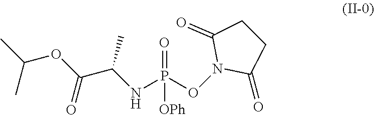 [SMILES: CC(C)OC(=O)[C@H](C)NP(=O)(Oc1ccccc1)ON1C(=O)CCC1=O]

with a compound of formula (III)

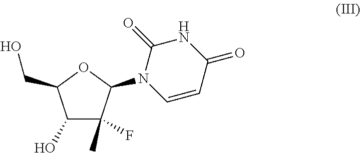 [SMILES: C[C@@]1(F)[C@H](O)[C@@H](CO)O[C@H]1n1ccc(=O)[nH]c1=O]

in the presence of a Lewis acid and a base,
   wherein the base comprises, preferably consists of, one or more of ethyldiisopropylamine, triethylamine, diethylamine, 1,8-diazabicycloundec-7-ene, pyridine, quinoline, isoquinoline, acridine, pyrazine, imidazole, benzimidazole, piperidine, tetramethylguanidine, ephedrine and pyrazole, more preferably consists of ethyldiisopropylamine, obtaining a mixture comprising the compound of formula (I),
   wherein the compound of formula (II-0) comprises a compound of formula (II-a)

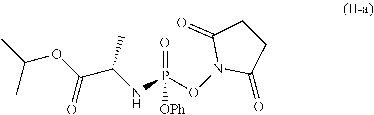 [SMILES: CC(C)OC(=O)[C@H](C)N[P@](=O)(Oc1ccccc1)ON1C(=O)CCC1=O]

and a compound of formula (II-b)

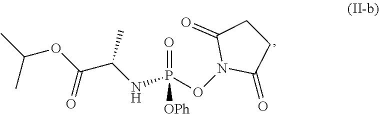 [SMILES: CC(C)OC(=O)[C@H](C)N[P@@](=O)(Oc1ccccc1)ON1C(=O)CCC1=O]

wherein the molar ratio of the compound of formula (II-a) relative the compound of formula (II-b) is in the range of from 45:55 to 72:28, preferably in the range of from 45:55 to 60:40, more preferably in the range of from 45:55 to 55:45. More preferably, the molar ratio of the compound of formula (II-a) relative the compound of formula (II-b) is in the range of from 54:46 to 46:54, more preferably in the range of from 53:47 to 47:53, more preferably in the range of from 52:48 to 48:52, more preferably in the range of from 51:49 to 49:51. More preferably, the molar ratio of the compound of formula (II-a) relative the compound of formula (II-b) is 1:1. More preferably, the compound of formula (II-0) consists of the compound of formula (II-a) and the compound of formula (II-b).

Therefore, the present invention preferably relates to a process for preparing a compound of formula (I)

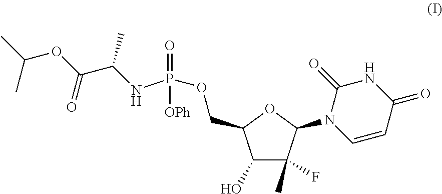 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a) reacting a compound of formula (II-0)

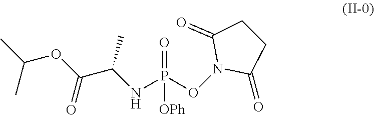 [SMILES: CC(C)OC(=O)[C@H](C)NP(=O)(Oc1ccccc1)ON1C(=O)CCC1=O]

with a compound of formula (III)

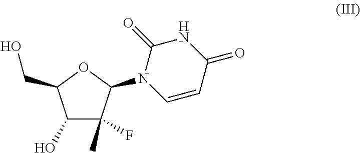 [SMILES: C[C@@]1(F)[C@H](O)[C@@H](CO)O[C@H]1n1ccc(=O)[nH]c1=O]

in the presence of a Lewis acid, preferably ZnBr₂ and a base, the base comprising, preferably consisting of, ethyldiisopropylamine or triethylamine, more preferably consisting of ethyldiisopropylamine and, obtaining a mixture comprising the compound of formula (I),
   wherein the compound of formula (II-0) comprises a compound of formula (II-a)

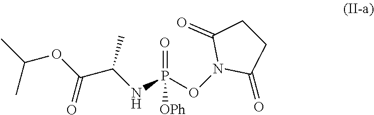 [SMILES: CC(C)OC(=O)[C@H](C)N[P@](=O)(Oc1ccccc1)ON1C(=O)CCC1=O]

and a compound of formula (II-b)

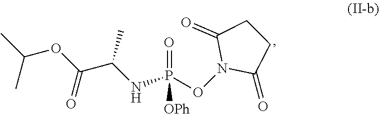 [SMILES: CC(C)OC(=O)[C@H](C)N[P@@](=O)(Oc1ccccc1)ON1C(=O)CCC1=O]

wherein the molar ratio of the compound of formula (II-a) relative the compound of formula (II-b) is in the range of from 45:55 to 72:28, preferably in the range of from 45:55 to 60:40, more preferably in the range of from 45:55 to 55:45. More preferably, the molar ratio of the compound of formula (II-a) relative the compound of formula (II-b) is in the range of from 54:46 to 46:54, more preferably in the range of from 53:47 to 47:53, more preferably in the range of from 52:48 to 48:52, more preferably in the range of from 51:49 to 49:51. More preferably, the molar ratio of the compound of formula (II-a) relative the compound of formula (II-b) is 1:1. More preferably, the compound of formula (II-0) consists of the compound of formula (II-a) and the compound of formula (II-b).

Therefore, the present invention relates to the process as defined above, wherein the compound of formula (II-0) employed in a) consists of the compound of formula (II-a) and the compound of formula (II-b), wherein the molar ratio of the compound of formula (II-a) relative the compound of formula (II-b) is 1:1.

Regarding methods to prepare the compound of formula (II), reference is made to examples 1.1 and 2.1 hereinbelow.

As mentioned above, the process of the present invention is characterized by an advantageous diastereoselectivity to the valuable product, the compound of formula (I-1), in particular the compound known as sofosbuvir. Thus, it is preferred that in the mixture obtained from a), the compound of formula (I) comprises a compound of formula (I-1)

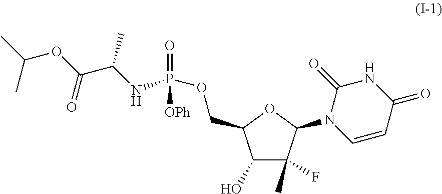 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and a compound of formula (I-2)

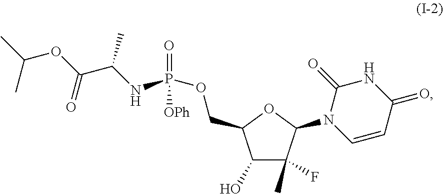 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

wherein the molar ratio of the compound of formula (I-1) relative to the compound of formula (I-2) is at least 65:35, preferably at least 70:30, more preferably at least 75:25, more preferably at least 80:20. It is conceivable that the molar ratio of the compound of formula (I-1) relative to the compound of formula (I-2) is at least 85:15 or at least 90:10 or at least 95:5 or at least 99:1 or at least 99.8:0.2. Preferably, the compound of formula (I) comprised in the mixture obtained from a) consists of the compound of formula (I-1) and the compound of formula (I-2).

It is conceivable that the molar ratio of the compound of formula (I-1) relative to the compound of formula (I-2) obtained in step a) is in the range of from 65:to 35 to 90:10, preferably in the range of from 75:25 to 90:10. It is conceivable that this molar ratio is further improved by crystallization, or crystallization and recrystallization, steps according to the present invention up to a molar ratio of the compound of formula (I-1) relative to the compound of formula (I-2) in the range of from 95:5 to 99.8:0.2, preferably in the range of from 99:1 to 99.8:0.2.

Therefore, the present invention relates to the process as defined above, wherein the compound of formula (II-0) employed in a) consists of the compound of formula (II-a) and the compound of formula (II-b), wherein the molar ratio of the compound of formula (II-a) relative the compound of formula (II-b) is 1:1, wherein the compound of formula (I) comprised in the mixture obtained from a) consists of the compound of formula (I-1) and the compound of formula (I-2), and wherein the molar ratio of the compound of formula (I-1) relative to the compound of formula (I-2) is in the range of from 75:25 to 90:10. It is preferred that after the crystallization of the compound of formula (I), the crystallized compound of formula (I) consists of the compound of formula (I-1) and the compound of formula (I-2), and wherein the molar ratio of the compound of formula (I-1) relative to the compound of formula (I-2) is in the range of from 95:5 to 99.8:0.2, preferably in the range of from 99:1 to 99.8:0.2.

Regarding the amount of the base relative to the amount of the compound of formula (III) employed in a), no specific restrictions exist. Preferably, prior to the reaction according to a), the molar ratio of the base relative to the compound of formula (III) is in the range of from 0.1:1 to 5:1. More preferably, prior to the reaction according to a), the molar ratio of the base relative to the compound of formula (III) is in the range of from 0.5:1 to 5:1, more preferably in the range of from 1:1 to 5:1, more preferably in the range of from 2:1 to 5:1. More preferably, prior to the reaction according to a), the molar ratio of the base relative to the compound of formula (III) is in the range of from 2:1 to 4:1, more preferably in the range of from 2.5:1 to 4:1, more preferably in the range of from 2.5:1 to 3.5:1. Also preferably, prior to the reaction according to a), the molar ratio of the base relative to the compound of formula (III) is in the range of from 1:1 to 3:1.

Therefore, the present invention preferably relates to a process for preparing a compound of formula (I)

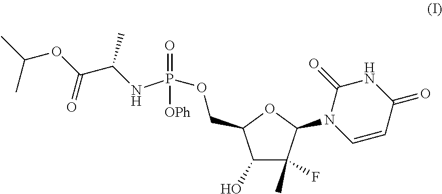 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a) reacting a compound of formula (II-0)

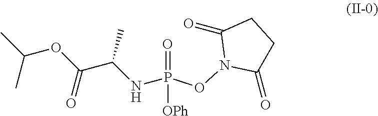 [SMILES: CC(C)OC(=O)[C@H](C)NP(=O)(Oc1ccccc1)ON1C(=O)CCC1=O]

with a compound of formula (III)

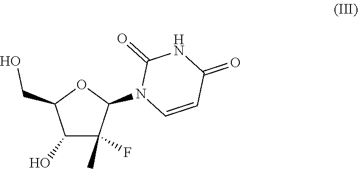 [SMILES: C[C@@]1(F)[C@H](O)[C@@H](CO)O[C@H]1n1ccc(=O)[nH]c1=O]

in the presence of a Lewis acid, preferably ZnBr₂ and a base, the base comprising, preferably consisting of, one or more of ethyldiisopropylamine, triethylamine, diethylamine, 1,8-diazabicycloundec-7-ene, pyridine, quinoline, isoquinoline, acridine, pyrazine, imidazole, benzimidazole, piperidine, tetramethylguanidine, ephedrine and pyrazole, said base more preferably comprising, more preferably consisting of ethyldiisopropylamine or trimethylamine, and obtaining a mixture comprising the compound of formula (I),
   wherein the compound of formula (II-0) comprises a compound of formula (II-a)

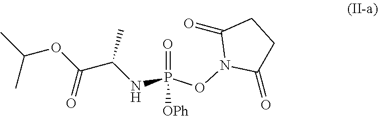 [SMILES: CC(C)OC(=O)[C@H](C)N[P@](=O)(Oc1ccccc1)ON1C(=O)CCC1=O]

and a compound of formula (II-b)

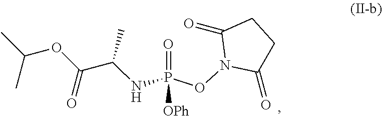 [SMILES: CC(C)OC(=O)[C@H](C)N[P@@](=O)(Oc1ccccc1)ON1C(=O)CCC1=O]

wherein the molar ratio of the compound of formula (II-a) relative the compound of formula (II-b) is in the range of from 45:55 to 72:28, preferably in the range of from 45:55 to 60:40, more preferably in the range of from 45:55 to 55:45.

More preferably, the molar ratio of the compound of formula (II-a) relative the compound of formula (II-b) is in the range of from 55:45 to 45:55, preferably in the range of from 54:46 to 46:54, more preferably in the range of from 53:47 to 47:53, more preferably in the range of from 52:48 to 48:52, more preferably in the range of from 51:49 to 49:51. More preferably, the molar ratio of the compound of formula (II-a) relative the compound of formula (II-b) is 1:1. More preferably, the compound of formula (II-0) consists of the compound of formula (II-a) and the compound of formula (II-b).

According to the present invention, it was found that the diastereoselectivity to the compound of formula (I-1), in particular the compound

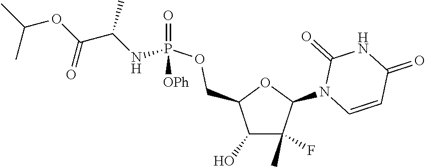 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

is achieved if the compound of formula (III) is reacted with the compound of formula (II-0) in the presence of the base and a Lewis acid.

No specific restrictions exist with regard to the chemical nature of the Lewis acid employed in a). Preferably, the Lewis acid comprises a twice positively charged ion or a three times positively charged ion, more preferably a twice positively charged metal ion or a three times positively charged metal ion. Generally, it is also conceivable that the Lewis acid comprises a twice positively charged ion and a three times positively charged ion, preferably a twice positively charged metal ion and a three times positively charged metal ion. With regard to the twice positively charged ion, it is preferred that it comprises, more preferably is, a Zn ion, a Mg ion, a Cu ion, or an Fe ion. More preferably, the twice positively charged ion comprises, more preferably is, a Zn ion or an Mg ion. More preferably, the twice positively charged ion comprises, more preferably is, a Zn ion. With regard to the three times positively charged ion, it is preferred that it comprises, more preferably is, a Mn ion.

Regarding the Lewis acid comprising a twice positively charged ion comprising, more preferably being, a Zn ion, no specific restrictions exist. Preferred Lewis acids comprise, more preferably are, Zn halides. More preferably, the Lewis acid comprises, preferably is, one or more of ZnBr₂, ZnCl₂, and ZnI₂. More preferably, the Lewis acid comprises, preferably is, ZnBr₂.

It is also conceivable that the Lewis acid is one or more of ZnBr₂, ZnCl₂, ZnI₂, MgBr₂, MgBr₂.OEt₂, CuCl₂, Cu(acetylacetonate)₂, and Fe(II) fumarate.

Regarding the Lewis acid comprising a three times positively charged ion comprising, preferably being, a Mn ion, no specific restrictions exist. Preferred Lewis acids comprise, more preferably are, Mn(acetylacetonate)₃.

Regarding the amount of the Lewis acid relative to the amount of the compound of formula (III) employed in a), no specific restrictions exist. Preferably, prior to the reaction according to a), the molar ratio of the Lewis acid relative to the compound of formula (III) is in the range of from 0.1:1 to 5:1. More preferably, prior to the reaction according to a), the molar ratio of the Lewis acid relative to the compound of formula (III) is in the range of from 0.2:1 to 5:1, preferably in the range of from 0.5:1 to 3:1, more preferably in the range of from 0.75:1 to 1.5:1, more preferably in the range of from 0.75:1 to 1.25:1.

Thus, the present invention preferably relates to a process for preparing a compound of formula (I)

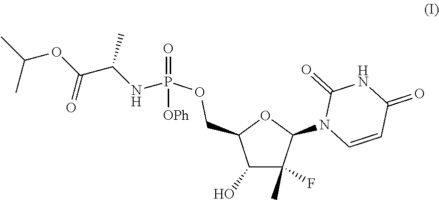 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a) reacting a compound of formula (II-0)

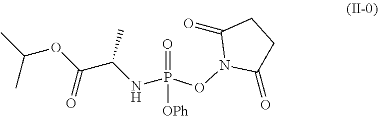 [SMILES: CC(C)OC(=O)[C@H](C)NP(=O)(Oc1ccccc1)ON1C(=O)CCC1=O]

with a compound of formula (III)

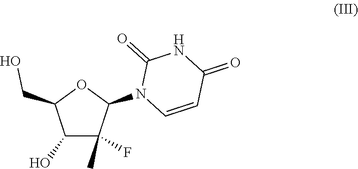 [SMILES: C[C@@]1(F)[C@H](O)[C@@H](CO)O[C@H]1n1ccc(=O)[nH]c1=O]

in the presence of a Lewis acid preferably comprising a Zn ion, more preferably comprising, more preferably being, ZnBr₂, and in the presence of a base comprising, preferably consisting of, one or more of ethyldiisopropylamine, triethylamine, diethylamine, 1,8-diazabicycloundec-7-ene, pyridine, quinoline, isoquinoline, acridine, pyrazine, imidazole, benzimidazole, piperidine, tetramethylguanidine, ephedrine and pyrazole, said base more preferably comprising, more preferably consisting of, ethyldiisopropylamine or trimethylamine, obtaining a mixture comprising the compound of formula (I), wherein the compound of formula (II-0) comprises a compound of formula (II-a)

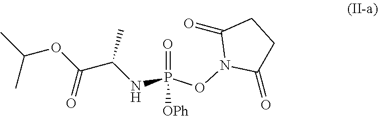 [SMILES: CC(C)OC(=O)[C@H](C)N[P@](=O)(Oc1ccccc1)ON1C(=O)CCC1=O]

and a compound of formula (II-b)

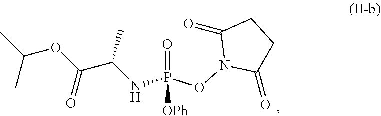 [SMILES: CC(C)OC(=O)[C@H](C)N[P@@](=O)(Oc1ccccc1)ON1C(=O)CCC1=O]

wherein the molar ratio of the compound of formula (II-a) relative the compound of formula (II-b) is in the range of from 45:55 to 72:28, preferably in the range of from 45:55 to 60:40, more preferably in the range of from 45:55 to 55:45. More preferably, the molar ratio of the compound of formula (II-a) relative the compound of formula (II-b) is in the range of from 54:46 to 46:54, more preferably in the range of from 53:47 to 47:53, more preferably in the range of from 52:48 to 48:52, more preferably in the range of from 51:49 to 49:51. More preferably, the molar ratio of the compound of formula (II-a) relative the compound of formula (II-b) is 1:1. More preferably, the compound of formula (II-0) consists of the compound of formula (II-a) and the compound of formula (II-b).

Regarding the amount of the compound of formula (II) employed in a) relative to the amount of the compound of formula (III) employed in a), no specific restrictions exist. Preferably, prior to the reaction according to a), the molar ratio of the compound of formula (II) relative to the compound of formula (III) is in the range of from 0.5:1 to 5:1, preferably in the range of 0.5 to 6:1. More preferably, prior to the reaction according to a), the molar ratio of the compound of formula (II) relative to the compound of formula (III) is in the range of from 0.6:1 to 4:1, preferably in the range of from 0.7:1 to 3:1. More preferably, prior to the reaction according to a), the molar ratio of the compound of formula (II) relative to the compound of formula (III) is in the range of from 0.8:1 to 2:1, preferably in the range of from 0.9:1 to 1.2:1.

It is further conceivable that the molar ratio of the compound of formula (II) relative to the compound of formula (III) is in the range of from 1.4:1 to 6:1, preferably in the range of from 1.4:1 to 4.9:1, more preferably in the range of from 2.1:1 to 5.5:1; more preferably in the range of from 2.1:1 to 4.9:1, more preferably in the range of from 3:1 to 5:1; 3:1 to 4.9:1, more preferably in the range of from 3:1 to 4:1.

It has been seen that a certain excess of the compound of formula (II) relative to the compound of formula (III) may further favour the resolution of compound of formula (I-1) thereby increasing the molar ratio of the compound of formula (I-1) relative to the compound of formula (I-2).

Thus, the present invention preferably relates to a process for preparing a compound of formula (I)

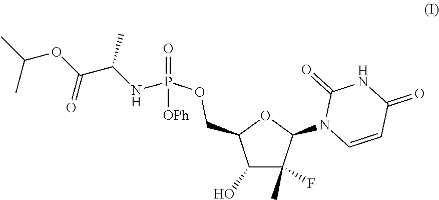 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a) reacting a compound of formula (II-0)

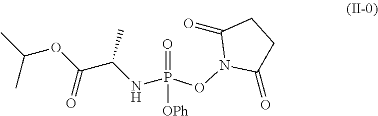 [SMILES: CC(C)OC(=O)[C@H](C)NP(=O)(Oc1ccccc1)ON1C(=O)CCC1=O]

with a compound of formula (III)

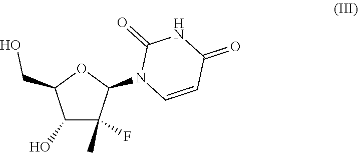 [SMILES: C[C@@]1(F)[C@H](O)[C@@H](CO)O[C@H]1n1ccc(=O)[nH]c1=O]

in the presence of a Lewis acid preferably comprising a Zn ion, more preferably comprising, more preferably being, ZnBr₂, and in the presence of a base comprising, preferably consisting of, one or more of ethyldiisopropylamine, triethylamine, diethylamine, 1,8-diazabicycloundec-7-ene, pyridine, quinoline, isoquinoline, acridine, pyrazine, imidazole, benzimidazole, piperidine, tetramethylguanidine, ephedrine and pyrazole, said base more preferably comprising, more preferably consisting of, ethyldiisopropylamine or trimethylamine, obtaining a mixture comprising the compound of formula (I),
   wherein the compound of formula (II-0) comprises a compound of formula (II-a)

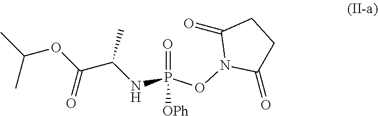 [SMILES: CC(C)OC(=O)[C@H](C)N[P@](=O)(Oc1ccccc1)ON1C(=O)CCC1=O]

and a compound of formula (II-b)

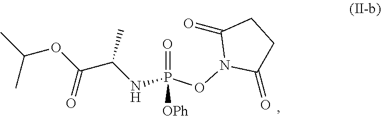 [SMILES: CC(C)OC(=O)[C@H](C)N[P@@](=O)(Oc1ccccc1)ON1C(=O)CCC1=O]

wherein the molar ratio of the compound of formula (II-a) relative the compound of formula (II-b) is in the range of from 45:55 to 55:45, preferably the molar ratio of the compound of formula (II-a) relative the compound of formula (II-b) is 1:1;
wherein prior to the reaction according to a), the molar ratio of the base relative to the compound of formula (III) is in the range of from 0.1:1 to 5:1, preferably in the range of from 2.5:1 to 4.5:1, more preferably in the range of from 2.5:1 to 4:1, more preferably in the range of from 2.5:1 to 3.5:1; more preferably, prior to the reaction according to a), the molar ratio of the base relative to the compound of formula (III) is in the range of from 1:1 to 3:1;
wherein prior to the reaction according to a), the molar ratio of the Lewis acid relative to the compound of formula (III) is in the range of from 0.5:1 to 3:1, preferably in the range of from 0.75:1 to 1.5:1;
wherein prior to the reaction according to a), the molar ratio of the compound of formula (II) relative to the compound of formula (III) is in the range of from 0.9:1 to 1.2:1; wherein the compound of formula (I) comprises a compound of formula (I-1) and a compound of formula (I-2) wherein molar ratio of the compound of formula (I-1) relative to the compound of formula (I-2) is at least 65:35, preferably at least 70:30, more preferably at least 75:25, more preferably at least 80:20. It is conceivable that the molar ratio of the compound of formula (I-1) relative to the compound of formula (I-2) is at least 85:15 or at least 90:10 or at least 95:5 or at least 99:1 or at least 99.8:0.2; more preferably the molar ratio of the compound of formula (I-1) relative to the compound of formula (I-2) in the range of from 95:5 to 99.8:0.2, preferably in the range of from 99:1 to 99.8:0.2; More preferably, the compound of formula (I) comprised in the mixture obtained from a) consists of the compound of formula (I-1) and the compound of formula (I-2).

Thus, the present invention preferably relates to a process for preparing a compound of formula (I)

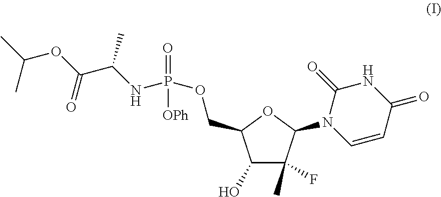 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a) reacting a compound of formula (II-0)

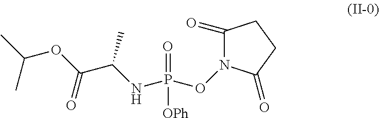 [SMILES: CC(C)OC(=O)[C@H](C)NP(=O)(Oc1ccccc1)ON1C(=O)CCC1=O]

with a compound of formula (III)

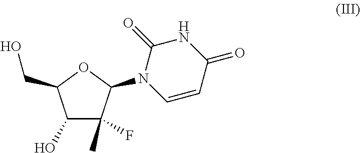 [SMILES: C[C@@]1(F)[C@H](O)[C@@H](CO)O[C@H]1n1ccc(=O)[nH]c1=O]

in the presence of a Lewis acid preferably comprising a Zn ion, more preferably comprising, more preferably being, ZnBr₂, and in the presence of a base comprising, preferably consisting of, one or more of ethyldiisopropylamine, triethylamine, diethylamine, 1,8-diazabicycloundec-7-ene, pyridine, quinoline, isoquinoline, acridine, pyrazine, imidazole, benzimidazole, piperidine, tetramethylguanidine, ephedrine and pyrazole, said base more preferably comprising, more preferably consisting of, ethyldiisopropylamine or trimethylamine, obtaining a mixture comprising the compound of formula (I),
   wherein the compound of formula (II-0) comprises a compound of formula (II-a)

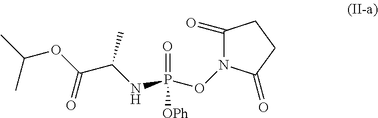 [SMILES: CC(C)OC(=O)[C@H](C)N[P@](=O)(Oc1ccccc1)ON1C(=O)CCC1=O]

and a compound of formula (II-b)

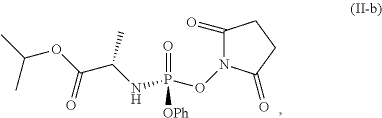 [SMILES: CC(C)OC(=O)[C@H](C)N[P@@](=O)(Oc1ccccc1)ON1C(=O)CCC1=O]

wherein the molar ratio of the compound of formula (II-a) relative the compound of formula (II-b) is in the range of from 45:55 to 55:45, preferably the molar ratio of the compound of formula (II-a) relative the compound of formula (II-b) is 1:1;
wherein prior to the reaction according to a), the molar ratio of the base relative to the compound of formula (III) is in the range of from 0.1:1 to 5:1, preferably in the range of from 2.5:1 to 4.5:1, more preferably in the range of from 2.5:1 to 4:1, more preferably in the range of from 2.5:1 to 3.5:1; more preferably, prior to the reaction according to a), the molar ratio of the base relative to the compound of formula (III) is in the range of from 1:1 to 3:1;
wherein prior to the reaction according to a), the molar ratio of the Lewis acid relative to the compound of formula (III) is in the range of from 0.5:1 to 3:1, preferably in the range of from 0.75:1 to 1.5:1;
wherein prior to the reaction according to a), the molar ratio of the compound of formula (II) relative to the compound of formula (III) is in the range of from 1.4:1 to 6:1, preferably in the range of from 1.4:1 to 4.9:1, more preferably in the range of from 2.1:1 to 5.5:1; more preferably in the range of from 2.1:1 to 4.9:1, more preferably in the range of from 3:1 to 5:1; 3:1 to 4.9:1, more preferably in the range of from 3:1 to 4:1.

Generally, it may be conceivable that the reacting according to a) is carried in the absence of an additional solvent. According to the present invention, it is preferred that according to a), the compound of formula (II-0) is reacted with the compound of formula (III) in the presence of the base, in the presence of the Lewis acid, and in the presence of a solvent.

Preferably, the solvent comprises, preferably is, one or more organic solvents, preferably one or more aprotic organic solvents. Generally, every aprotic organic solvent can be employed which allows to carry out the reacting according to a). Preferably, the aprotic organic solvent Comprises, more preferably consists of, one or more of dichloromethane, methyl tert-butyl ether, tetrahydrofuran, dimethylsulphoxide, and dimethylformamide. More preferably, the solvent comprises, preferably is, tetrahydrofuran.

Thus, the present invention preferably relates to a process for preparing a compound of formula (I)

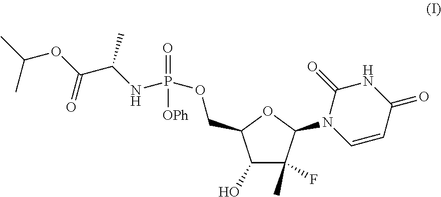 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a) reacting a compound of formula (II-0)

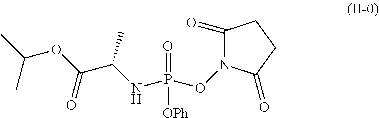 [SMILES: CC(C)OC(=O)[C@H](C)NP(=O)(Oc1ccccc1)ON1C(=O)CCC1=O]

with a compound of formula (III)

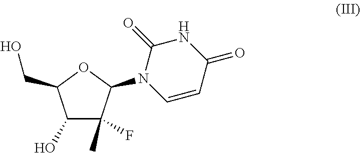 [SMILES: C[C@@]1(F)[C@H](O)[C@@H](CO)O[C@H]1n1ccc(=O)[nH]c1=O]

in the presence of a solvent, preferably being tetrahydrofuran, of a Lewis acid preferably being ZnBr₂ and of a base preferably being ethyldiisopropylamine or trimethylamine, obtaining a mixture comprising the compound of formula (I),
   wherein the compound of formula (II-0) comprises a compound of formula (II-a)

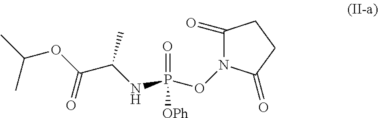 [SMILES: CC(C)OC(=O)[C@H](C)N[P@](=O)(Oc1ccccc1)ON1C(=O)CCC1=O]

and a compound of formula (II-b)

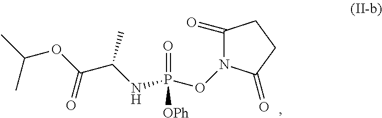 [SMILES: CC(C)OC(=O)[C@H](C)N[P@@](=O)(Oc1ccccc1)ON1C(=O)CCC1=O]

wherein the molar ratio of the compound of formula (II-a) relative the compound of formula (II-b) is 1:1, wherein prior to the reaction according to a), the molar ratio of the base relative to the compound of formula (III) is in the range of from 1:1 to 3:1, the molar ratio of the Lewis acid relative to the compound of formula (III) is in the range of from 0.75:1 to 1.5:1, and the molar ratio of the compound of formula (II) relative to the compound of formula (III) is in the range of from 3:1 to 4.9:1, preferably in the range of from 3:1 to 4:1.

The temperature at which the reacting according to a) is carried out can be suitably chosen, depending on the chemical nature of components of the mixture which is subjected to reaction conditions according to a), and in particular, if present, the chemical nature of the solvent. Preferably, the reacting according to a) is carried out at a temperature in the range of from 0 to 80° C., preferably in the range of from 0 to 70° C., more preferably in the range of from 0 to 60° C., more preferably in the range of from 0 to 50° C., more preferably in the range of from 0 to 50° C., more preferably in the range of from 0 to 40° C., more preferably in the range of from 0 to 30° C., more preferably in the range of from 0 to 25° C. More preferably, the reacting according to a) is carried out at a temperature in the range of from 0 to 20° C., more preferably in the range of from 0 to 15° C., more preferably in the range of from 0 to 10° C., more preferably in the range of from 0 to 5° C.

It has been observed that the dynamic resolution occurs at any of the above disclosed temperatures. Room temperature further favors the dynamic resolution. A temperature lower than 15° C., preferably a temperature of 10° C. or less can further increase the efficiency of the process of dynamic resolution.

Generally, the compounds subjected to reacting in a) can be admixed in any sequence. Preferably, the compound of formula (II) is admixed with the compound of formula (III) wherein, if a solvent is used, the compound of formula (II) can be preferably employed dissolved in this solvent; it is further preferred that to the resulting mixture, the Lewis acid is added; it is further preferred that the resulting mixture is then cooled to a temperature in the range of from 0 to 25° C., preferably in the range of from 0 to 15° C., more preferably in the range of from 0 to 10° C., more preferably in the range of from 0 to 5° C. and to the thus cooled mixture, the base is added. After the addition of the base, the temperature is allowed to rise. For example if the addition of the base occurs at 0° C., the temperature is allowed to rise to reach a temperature in the range of from 15 to 25° C.

The period of time for which the reacting according to a) is carried out can be suitably chosen. Preferably, the reacting according to a) is carried out for a period of time in the range of from 0.5 to 48 h, preferably in the range of from 0.75 to 42 h, more preferably in the range of from 1 to 36 h. More preferably, the reacting according to a) is carried out for a period of time in the range of from 1.5 to 20 h, preferably in the range of from 2 to 24 h. Preferred ranges are from 2 to 6 h or from 6 to 10 h or from 10 to 14 h or from 14 to 19 h or from 19 to 24 h. The period of time for which the reacting according to a) is carried out is more preferably in the range of 15 to 24 h.

Preferably, during reacting according to a), the reaction mixture is agitated, preferably mechanically agitated, more preferably stirred. The term “agitation” as used herein relates to any motion of a macroscopic constituent of the reaction mixture which is induced from outside, relative to another macroscopic constituent of the reaction mixture. The term “mechanical agitation” as used herein relates to any motion of a macroscopic constituent of the reaction mixture which is induced from outside via a device, such as shaking or stirring or sonication, relative to another macroscopic constituent of the reaction mixture. The term “stirring” as used herein relates to any motion of a macroscopic constituent of the reaction mixture which is induced from outside via a stirring device, relative to another macroscopic constituent of the reaction mixture.

### Step b)

Preferably, the compound of formula (I) comprised in the mixture obtained from a) is suitably separated from said mixture. Generally, it is preferred that from said separating, a composition is obtained which comprises the compound of formula (I), in particular a composition comprising the compound of formula (I) comprising the compound of formula (I-1) and the compound of formula (I-1), preferably a composition comprising the compound of formula (I) consisting of the compound of formula (I-1) and the compound of formula (I-2). Therefore, the present invention also relates to the process as defined above, further comprising b) separating the compound of formula (I) from the mixture obtained in a).

It is preferred that the compound of formula (I) is separated from the liquid phase of the mixture obtained in a) wherein the separating preferably includes filtration or centrifugation, more preferably filtration. Further, it is preferred that the compound of formula (I) obtained from filtration or centrifugation, preferably filtration, is washed and/or dried, preferably washed and dried. No specific limitations exist regarding the chemical nature of the washing agent. Preferred washing agents include isopropyl acetate. No specific limitations exist for the drying conditions. Preferred drying conditions include a pressure below 1 bar, preferably drying in vacuo. Further, it is preferred that the compound of formula (I), preferably after drying, is further dissolved in one or more solvents, preferably in a solvent used for crystallization and recrystallization as disclosed below.

The thus dissolved compound of formula (I) can be further subjected to extraction, including, for example, extraction with aqueous sodium chloride, obtaining an organic phase from which the solvent is preferably removed whereafter the solid compound of formula (I) is preferably dissolved in one or more further solvents. If extraction is carried, it is, for example, preferred to dissolve the compound of formula (I), after separation from the liquid phase of the mixture obtained in a) and drying, in a first organic solvent, for example, isopropyl acetate, subject the thus obtained solution to extraction, for example with aqueous sodium chloride, obtaining an organic phase from which the solvent is suitably removed, and dissolve the thus obtained solid compound of formula (I) in a second organic solvent, for example toluene.

Therefore, the present invention also relates to the process as defined above, preferably further comprising
b) separating the compound of formula (I) from the mixture obtained in a), obtaining the compound of formula (I) dissolved in a solvent, preferably an organic solvent.

Further Steps

It is preferred that in the mixture obtained from b) preferably comprising the compound of formula (I) dissolved in a solvent, preferably an organic solvent, the compound of formula (I-1) is suitably crystallized. From this crystallization, the compound of formula (I) (I) is obtained in its mother liquor from which it is preferably suitably separated.

Therefore, the present invention also relates to the process as defined above, further comprising
c) crystallizing the compound of formula (I), preferably from the mixture obtained from b) comprising the compound of formula (I) dissolved in a solvent.

Further, the present invention also relates to the process as defined above, further comprising
c) crystallizing the compound of formula (I), preferably from the mixture obtained from b) comprising the compound of formula (I) dissolved in a solvent, obtaining the crystallized compound of formula (I) comprised in its mother liquor;
d) separating the crystallized compound of formula (I) from its mother liquor; and
e) optionally recrystallizing the compound of formula (I) from d) from a solvent, preferably an organic solvent,
f) optionally separating the crystallized compound of formula (I) from e) from the solvent.

The compound of formula (I) after c) or after e) or after f) comprises a compound of formula (I-1)

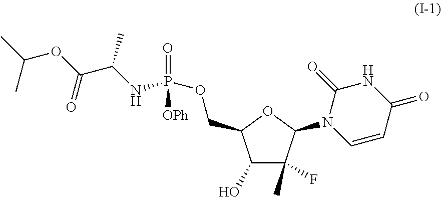 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and a compound of formula (I-2)

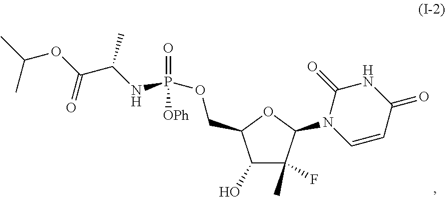 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

wherein the molar ratio of the compound of formula (I-1) relative to the compound of formula (I-2) is at least 65:35, preferably at least 70:30, more preferably at least 75:25, more preferably at least 80:20. It is conceivable that the molar ratio of the compound of formula (I-1) relative to the compound of formula (I-2) is at least 85:15 or at least 90:10 or at least 95:5 or at least 99:1 or at least 99.8:0.2. More preferably, the compound of formula (I) comprised in the mixture obtained from a) consists of the compound of formula (I-1) and the compound of formula (I-2).

It is conceivable that the molar ratio of the compound of formula (I-1) relative to the compound of formula (I-2) obtained in step a) and after b) is in the range of from 65:to 35 to 90:10, preferably in the range of from 75:25 to 90:10. It is conceivable that this molar ratio is further improved by crystallization step c) or crystallization step c) and recrystallization step e) up to a molar ratio of the compound of formula (I-1) relative to the compound of formula (I-2) in the range of from 95:5 to 99.8:0.2, preferably in the range of from 99:1 to 99.8:0.2.

Hence, it is preferred that the molar ratio of the compound of formula (I-1) relative to the compound of formula (I-2) after c) or e) is increased with respect to the molar ratio of the compound of formula (I-1) relative to the compound of formula (I-2) of a) and of b). In other words, the crystallization step and the recrystallization steps may further improve the molar ratio of the compound of formula (I-1) relative to the compound of formula (I-2). The molar ratio of the compound of formula (I-1) relative to the compound of formula (I-2) from d) or from e) i.e. after the crystallization or after recrystallization is at least 95:5, preferably at least 97:3, more preferably at least 99:1, more preferably 99.8:0.2. The molar ratio of the compound of formula (I-1) relative to the compound of formula (I-2) from d) or from e) is in the range of from 95:5 to 99.8:0.2, in the range of from 97:3 to 99.8:0.2, more preferably in the range of from 99:1 to 99.8:0.2, more preferably is 99.8:0.2.

During crystallization in c), it can be preferred that suitable seed crystals are added, preferably seed crystals of the compound of formula (I-1).

During recrystallization in e), it can be preferred that suitable seed crystals are added, preferably seed crystals of the compound of formula (I-1).

After the crystallization in c) from which the crystallized compound of formula (I) comprising compounds (I-1) and (1-2) in the molar ratio disclosed above is obtained in its mother liquor, the crystallized compound of formula (I) comprising compounds (I-1) and (1-2) in the molar ratio disclosed above is preferably suitably separated from its mother liquor in d), for example by filtration or centrifugation. The thus separated crystallized compound of formula (I) comprising compounds (I-1) and (1-2) in the molar ratio disclosed above can be subjected to washing, wherein preferred washing agents include a solvent as disclosed below, and subject the optionally washed crystallized compound of formula (I) to drying. Preferred drying conditions include temperatures in the range of from 10 to 60° C., preferably in the range of from 30 to 50° C., and a pressure below ambient pressure.

Therefore, the present invention also relates to the process as defined above, further comprising
c) crystallizing the compound of formula (I), preferably from the mixture obtained from b) comprising the compound of formula (I) dissolved in a solvent, obtaining the crystallized compound of formula (I) in its mother liquor;
d) separating the crystallized compound of formula (I) from its mother liquor, obtaining the compound of formula (I) in crystalline form, said separating comprising
   d1) subjecting the mother liquor comprising the crystallized compound of formula (I) to filtration, obtaining a filter cake comprising the compound of formula (I);
   d2) optionally washing the filter cake;
   d3) drying the optionally washed filter cake, obtaining the compound of formula (I);
e) optionally recrystallizing the compound of formula (I) from d) from a solvent, preferably an organic solvent;
f) optionally separating the crystallized compound of formula (I) from e) from the solvent.

The compound of formula (I) comprises, preferably consists of a compound of formula (I-1) and a compound of formula (I-2). It is preferred that the molar ratio of the compound of formula (I-1) relative to the compound of formula (I-2) after c) or from d) or from d3) or from e) i.e. after the crystallization or recrystallization step is increased with respect to the molar ratio of the compound of formula (I-1) relative to the compound of formula (I-2) from a). In other words, the crystallization step and the recrystallization step may further improve the molar ratio of the compound of formula (I-1) relative to the compound of formula (I-2). The molar ratio of the compound of formula (I-1) relative to the compound of formula (I-2) after c) or from d) or from d3) or from e) i.e. after the crystallization or recrystallization step is at least 95:5, preferably at least 97:3, more preferably at least 99:1, more preferably 99.8:0.2. The molar ratio of the compound of formula (I-1) relative to the compound of formula (I-2) is in the range of from 95:5 to 99.8:0.2, preferably in the range of from 97:3 to 99.8:0.2, more preferably in the range of from 99:1 to 99.8:0.2, more preferably is 99.8:0.2.

For the purpose of obtaining the crystalline compound of formula (I) comprising compounds (I-1) and (1-2) with the molar ratio as disclosed above, it is preferred that the crystallization step c) and the recrystallization step of e) is carried in a solvent, preferably in an organic solvent, more preferably an organic solvent selected from the group consisting of a ketone, an ester, an ether, a C₁-C₇ alkane, a halo-alkane, a nitrile, an aromatic hydrocarbon solvent, an alcohol or a mixture thereof.

Regarding the ketone, it is preferably selected from the group consisting of acetone, methyl isobutyl ketone, diethyl ketone, methyl propyl ketone, methyl isopropyl ketone, acetophenone, and diethyl butyl ketone, optionally in combination with a solvent selected from the group consisting of methyl tert-butyl ether, isopropyl acetate, and toluene.

Regarding the ether, it is preferably selected from the group consisting of methyl tert-butyl ether and tetrahydrofuran.

Regarding the ester, it is preferably selected from the group consisting of ethyl acetate, isopropyl acetate and butyl acetate.

Regarding the C₁-C₇ alkane, it is preferably selected from a C₅-C₇ alkane wherein the C₅-C₇ alkane is selected from the group consisting of cyclohexane and n-heptane, optionally in combination with a solvent selected from the group consisting of methyl tert-butyl ether, isopropyl acetate and toluene.

Regarding the halo-alkane, it is preferably dichloromethane, optionally in combination with a solvent selected from the group consisting of toluene, tetrahydrofuran, acetone, and methyl isobutyl ketone.

Regarding the nitrile, it is preferably acetonitrile, optionally in combination with a solvent selected from the group consisting of diisopropyl ether and tert-butyl methyl ether.

Regarding the aromatic hydrocarbon solvent, it is preferably selected from the group consisting of anisole and toluene.

Regarding the alcohol it is preferably selected from a C₁-C₈ alcohol, more preferably the alcohol is n-butanol, optionally in combination with heptane.

Hence, it is preferred that the solvent of steps c) and e) is selected from the group consisting of acetone, methyl isobutyl ketone, diethyl ketone, methyl propyl ketone, methyl isopropyl ketone, acetophenone, diethyl butyl ketone, methyl tert-butyl ether, tetrahydrofuran, ethyl acetate, isopropyl acetate, butyl acetate, cyclohexane, a heptane, preferably n-heptane, dichloromethane, acetonitrile, anisole, toluene, n-butanol and a mixture thereof. More preferably the solvent is dichloromethane.

The solvents for crystallization are preferably also used as washing agents.

For the purpose of obtaining the crystalline compound of formula (I) with a molar ratio of the compound of formula (I-1) relative to the compound of formula (I-2) improved with respect to the molar ratio of compound of formula (I-1) relative to the compound of formula (I-2) after a), it preferred that the crystallization step c) and the recrystallization step of e) (step e) if carried out) is carried in a solvent, preferably an organic solvent selected from the group of dichloromethane, acetonitrile and anisole or mixture thereof, wherein more preferably the solvent is dichloromethane. In this case, after c) or after e), the molar ratio of the compound of formula (I-1) relative to the compound of formula (I-2) is preferably at least 95:5, more preferably at least 97:3, more preferably at least 99:1, more preferably 99.8:0.2. In this case after c) or after e) the molar ratio of the compound of formula (I-1) relative to the compound of formula (I-2) is in the range of from 95:5 to 99.8:0.2, preferably in the range of from 97:3 to 99.8:0.2, more preferably in the range of from 99:1 to 99.8:0.2, more preferably is 99.8:0.2. More preferably, the solvent is dichloromethane.

It is preferred that the crystallization step c) is carried out at a temperature range in the range of from −10 to 50° C.

It is preferred that recrystallization step of e) is carried out at a temperature range in the range of from −10 to 50° C.

### Mixtures and Uses

Generally, the present invention also relates to a mixture which is obtainable or obtained by the process of the present invention, preferably obtainable or obtained from step a) of a process of the present invention. More preferably, the present invention relates to a mixture which is obtainable or obtained from step a) of a process as defined above, step a) comprising reacting a compound of formula (II-0)

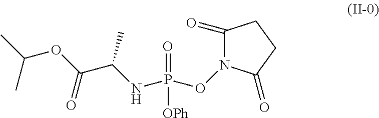 [SMILES: CC(C)OC(=O)[C@H](C)NP(=O)(Oc1ccccc1)ON1C(=O)CCC1=O]

with a compound of formula (III)

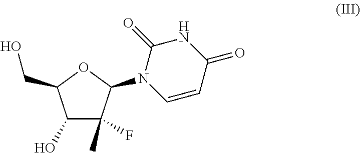 [SMILES: C[C@@]1(F)[C@H](O)[C@@H](CO)O[C@H]1n1ccc(=O)[nH]c1=O]

wherein the compound of formula (II-0) comprises a compound of formula (II-a)

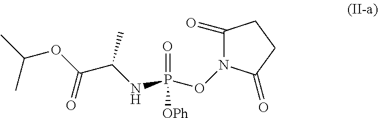 [SMILES: CC(C)OC(=O)[C@H](C)N[P@](=O)(Oc1ccccc1)ON1C(=O)CCC1=O]

and a compound of formula (II-b)

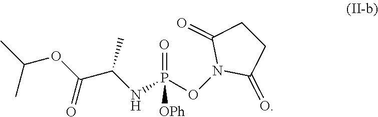 [SMILES: CC(C)OC(=O)[C@H](C)N[P@@](=O)(Oc1ccccc1)ON1C(=O)CCC1=O]

Yet further, the present invention relates to a mixture which may be obtainable or obtained from step a) of the process as defined above, wherein said mixture comprises the compound of formula (I) comprising a compound of formula (I-1)

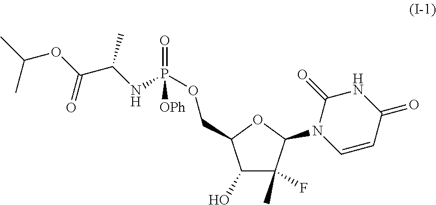 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and a compound of formula (I-2)

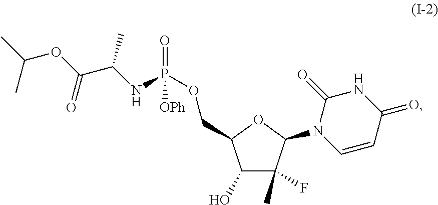 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

said mixture further comprising a base and a Lewis acid and wherein in the mixture, the molar ratio of the compound of formula (I-1) relative to the compound of formula (I-2) is at least 65:35, preferably at least 70:30, more preferably at least 75:25, more preferably at least 80:20. It is conceivable that in the mixture the molar ratio of the compound of formula (I-1) relative to the compound of formula (I-2) is at least 85:15 or at least 90:10 or at least 95:5 or at least 99:1 or at least 99.8:0.2. More preferably, the compound of formula (I) comprised in the mixture obtained from a) consists of the compound of formula (I-1) and the compound of formula (I-2). It is further conceivable that in the mixture the molar ratio of the compound of formula (I-1) relative to the compound of formula (I-2) is in the range of from 65:to 35 to 90:10, preferably in the range of from 75:25 to 90:10, more preferably in the range of from 95:5 to 99.8:0.2, more preferably in the range of from 99:1 to 99.8:0.2.

Regarding further preferred embodiments of said mixture, reference is made to the disclosure above, in particular regarding preferred molar ratios disclosed above and preferred bases and preferred Lewis acid. Further reference is made to the respective disclosure in the embodiment section of the present application. A preferred mixture of the present invention comprises the compound of formula (I) comprising a compound of formula (I-1)

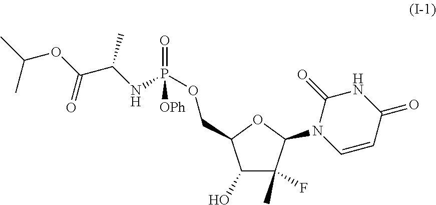 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and a compound of formula (I-2)

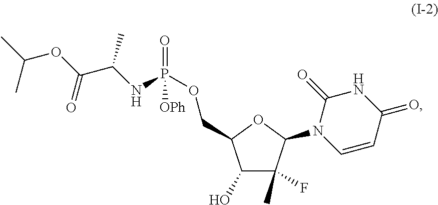 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

wherein in the mixture, the molar ratio of the compound of formula (I-1) relative to the compound of formula (I-2) is at least 85:15, said mixture further comprising a base which is ethyldiisopropylamine, a Lewis acid which is ZnBr₂, and preferably a solvent which is preferably tetrahydrofuran.

Preferably, this mixture is used for obtaining the compound of formula

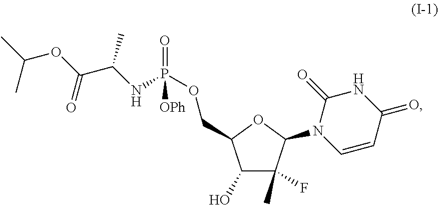 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

wherein obtaining the compound of formula (I-1) preferably comprises separating the compound of formula (I) from the mixture and crystallizing the compound of formula (I), obtaining the crystallized compound of formula (I) in its mother liquor and separating the crystallized compound of formula (I) from its mother liquor, wherein as mentioned above after the crystallization or recrystallization step the molar ratio of compound of formula (I-1) relative to the compound of formula (I-2) may be increased with respect to the molar ratio of the compound of formula (I-1) relative to the compound of formula (I-2) of a). In other words, the crystallization step and recrystallization steps further improve the molar ratio of the compound of formula (I-1) relative to the compound of formula (I-2). The molar ratio of the compound of formula (I-1) relative to the compound of formula (I-2) after the crystallization or recrystallization is at least 95:5, preferably at least 97:3, more preferably at least 99:1, more preferably is 99.8:0.2.

As mentioned above, the process of the present invention allows the diastereoselective preparation of the compound of formula (I-1) based on a phosphoramidate derivative having a succinimide group as the leaving group. This reaction is based on a specific starting mixture which is subjected to reaction conditions. Therefore, the present invention also relates to this novel mixture which comprises a compound of formula (II-0)

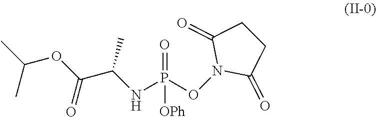 [SMILES: CC(C)OC(=O)[C@H](C)NP(=O)(Oc1ccccc1)ON1C(=O)CCC1=O]

and a compound of formula (III)

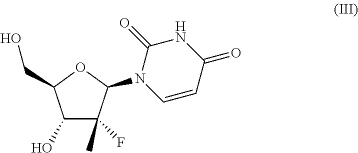 [SMILES: C[C@@]1(F)[C@H](O)[C@@H](CO)O[C@H]1n1ccc(=O)[nH]c1=O]

and a Lewis acid and a base, wherein the compound of formula (II-0) comprises a compound of formula (II-a)

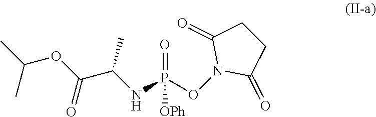 [SMILES: CC(C)OC(=O)[C@H](C)N[P@](=O)(Oc1ccccc1)ON1C(=O)CCC1=O]

and a compound of formula (II-b)

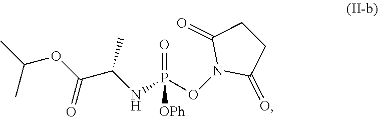 [SMILES: CC(C)OC(=O)[C@H](C)N[P@@](=O)(Oc1ccccc1)ON1C(=O)CCC1=O]

wherein the molar ratio of the compound of formula (II-a) relative the compound of formula (II-b) is in the range of from 45:55 to 72:28, preferably in the range of from 45:55 to 60:40, more preferably in the range of from 45:55 to 55:45.

Preferably, the molar ratio of the compound of formula (II-a) relative the compound of formula (II-b) is in the range of from 55:45 to 45:55, preferably in the range of from 54:46 to 46: 54, more preferably in the range of from 53:47 to 47:53, more preferably in the range of from 52:48 to 48:52, more preferably in the range of from 51:49 to 49:51. More preferably, the molar ratio of the compound of formula (II-a) relative the compound of formula (II-b) is 1:1. More preferably, the compound of formula (II-0) consists of the compound of formula (II-a) and the compound of formula (II-b).

Regarding further preferred embodiments of said mixture, reference is made to the disclosure above, in particular regarding preferred molar ratios disclosed above and preferred bases and preferred Lewis acid. Further reference is made to the respective disclosure in the embodiment section of the present application. A preferred mixture of the present invention comprises a compound of formula (II-0)

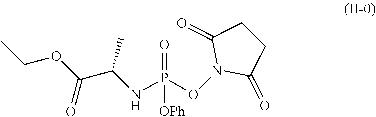 [SMILES: CCOC(=O)[C@H](C)NP(=O)(Oc1ccccc1)ON1C(=O)CCC1=O]

and a compound of formula (III)

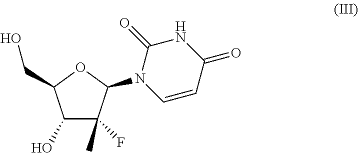 [SMILES: C[C@@]1(F)[C@H](O)[C@@H](CO)O[C@H]1n1ccc(=O)[nH]c1=O]

and a base which is ethyldiisopropylamine and a Lewis acid which is ZnBr₂, wherein the compound of formula (II-0) comprises a compound of formula (II-a)

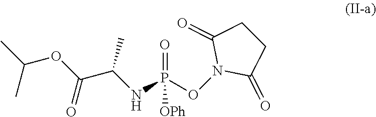 [SMILES: CC(C)OC(=O)[C@H](C)N[P@](=O)(Oc1ccccc1)ON1C(=O)CCC1=O]

and a compound of formula (II-b)

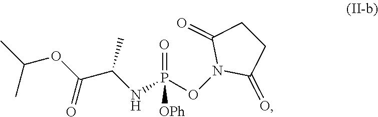 [SMILES: CC(C)OC(=O)[C@H](C)N[P@@](=O)(Oc1ccccc1)ON1C(=O)CCC1=O]

wherein the molar ratio of the compound of formula (II-a) relative the compound of formula (II-b) is in the range of from 45:55 to 72:28, preferably in the range of from 45:55 to 60:40, more preferably in the range of from 45:55 to 55:45; more preferably, the molar ratio of the compound of formula (II-a) relative the compound of formula (II-b) is 1:1; wherein the molar ratio of the base relative to the compound of formula (III) is in the range of from 1.5:1 to 2:1, the molar ratio of the compound of formula (II) relative to the compound of formula (III) is in the range of from 2:1 to 6:1, and the molar ratio of the Lewis acid relative to the compound of formula (III) is in the range of from 0.25 to 1.5:1, the mixture preferably comprising a solvent, more preferably tetrahydrofuran.

Preferably, this mixture is used for obtaining the compound of formula

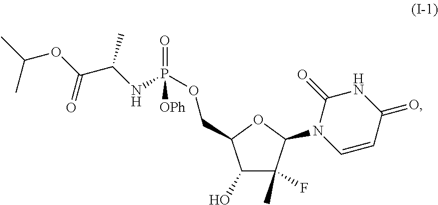 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

wherein obtaining the compound of formula (I-1) preferably comprises subjecting the mixture to reaction conditions obtaining a mixture comprising the compound of formula (I), separating the compound of formula (I) from the mixture and crystallizing the compound of formula (I-1), obtaining the crystallized compound of formula (I-1) in its mother liquor and separating the crystallized compound of formula (I-1) from its mother liquor.

As also mentioned above, the use of this mixture allows improving the diastereoselectivity to the compound of formula (I-1) when using the phosphoramidate derivative having a succinimide group as the leaving group as starting material. Thus, the present invention also relates to the use of this mixture for improving the selectivity of the reaction of a compound of formula (III) with a compound of formula (II-0) to the compound of formula (I-1)

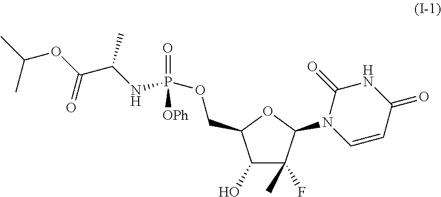 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

obtained from said reaction,
wherein the compound of formula (II-0) comprises a compound of formula (II-a) and a compound of formula (II-b), wherein the molar ratio of the compound of formula (II-a) relative the compound of formula (II-b) is in the range of from 45:55 to 72:28, preferably in the range of from 45:55 to 60:40, more preferably in the range of from 45:55 to 55:45; more preferably, the molar ratio of the compound of formula (II-a) relative the compound of formula (II-b) is in the range of from 55:45 to 45:55, preferably in the range of from 54:46 to 46:54, more preferably in the range of from 53:47 to 47:53, more preferably in the range of from 52:48 to 48:52, more preferably in the range of from 51:49 to 49:51; more preferably, the molar ratio of the compound of formula (II-a) relative the compound of formula (II-b) is 1:1. More preferably, the compound of formula (II-0) consists of the compound of formula (II-a) and the compound of formula (II-b).

Further, the present invention relates to a method for improving the selectivity of the reaction of a compound of formula (III) with a compound of formula (II-0) to the compound of formula (I-1)

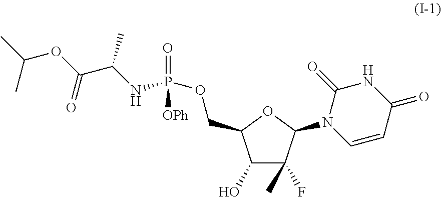 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

obtained from said reaction,
wherein the compound of formula (II-0) comprises a compound of formula (II-a) and a compound of formula (II-b), wherein the molar ratio of the compound of formula (II-a) relative the compound of formula (II-b) is in the range of from 45:55 to 72:28, preferably in the range of from 45:55 to 60:40, more preferably in the range of from 45:55 to 55:45; more preferably, the molar ratio of the compound of formula (II-a) relative the compound of formula (II-b) is in the range of from 55:45 to 45:55, preferably in the range of from 54:46 to 46:54, more preferably in the range of from 53:47 to 47:53, more preferably in the range of from 52:48 to 48:52, more preferably in the range of from 51:49 to 49:51; more preferably, the molar ratio of the compound of formula (II-a) relative the compound of formula (II-b) is 1:1; wherein more preferably, the compound of formula (II-0) consists of the compound of formula (II-a) and the compound of formula (II-b) and
wherein said method comprises employing the above-mentioned mixture as starting material in said reaction.

Generally, it was found that the Lewis acid and the base as disclosed above in combination lead to an increase of the diastereoselectivity of the compound of formula (I-1) relative to the compound of formula (I-2). It has been further seen that an excess of equivalents of compound (II) relative to compound (III) leads to an increase of the diastereoselectivity of the compound of formula (I-1) relative to the compound of formula (I-2). It was further found that a low temperature preferably a temperature lower than 15° C., more preferably in the range of from 10 to 0° C. further increases the diastereoselectivity of the reaction in favor of compound (I-1).

Generally, it was found that in particular an improved selectivity of the reaction of the compound of formula (II-0) with the compound of formula (III) to the compound of formula (I-1) is obtained when using a phosphoramidate derivative having a succinimide group as the leaving group as starting material.

Thus, the present invention also relates to the use of a combination of a Lewis acid and a base for improving the selectivity of the reaction of a compound of formula (III)

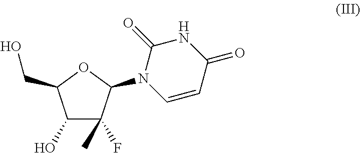 [SMILES: C[C@@]1(F)[C@H](O)[C@@H](CO)O[C@H]1n1ccc(=O)[nH]c1=O]

with a compound of formula (II-0)

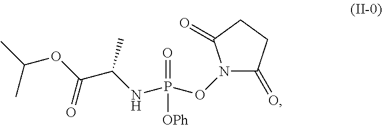 [SMILES: CC(C)OC(=O)[C@H](C)NP(=O)(Oc1ccccc1)ON1C(=O)CCC1=O]

to the compound of formula (I-1)

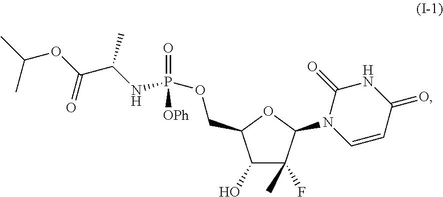 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

obtained from said reaction,
said compound of formula (II-0) comprising a compound of formula (II-a)

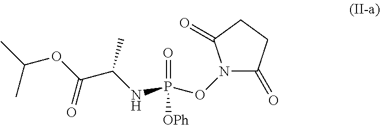 [SMILES: CC(C)OC(=O)[C@H](C)N[P@](=O)(Oc1ccccc1)ON1C(=O)CCC1=O]

and a compound of formula (II-b)

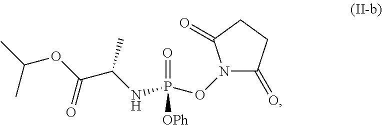 [SMILES: CC(C)OC(=O)[C@H](C)N[P@@](=O)(Oc1ccccc1)ON1C(=O)CCC1=O]

wherein the molar ratio of the compound of formula (II-a) relative the compound of formula (II-b) is in the range of from 45:55 to 72:28, preferably in the range of from 45:55 to 60:40, more preferably in the range of from 45:55 to 55:45; more preferably, the molar ratio of the compound of formula (II-a) relative the compound of formula (II-b) is in the range of from 55:45 to 45:55, preferably in the range of from 54:46 to 46:54, more preferably in the range of from 53:47 to 47:53, more preferably in the range of from 52:48 to 48:52, more preferably in the range of from 51:49 to 49:51; more preferably, the molar ratio of the compound of formula (II-a) relative the compound of formula (II-b) is 1:1; and wherein more preferably, the compound of formula (II-0) consists of the compound of formula (II-a) and the compound of formula (II-b).

Regarding further preferred combinations, reference is made to the disclosure above, in particular regarding preferred molar ratios disclosed above and preferred bases and preferred Lewis acids. Further reference is made to the respective disclosure in the embodiment section of the present application. According to preferred respective use of the present invention, the base is ethyldiisopropylamine and the Lewis acid is ZnBr₂. Further, the present invention relates to a method for improving the selectivity of the reaction of a compound of formula (III)

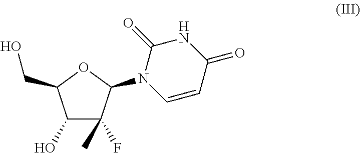 [SMILES: C[C@@]1(F)[C@H](O)[C@@H](CO)O[C@H]1n1ccc(=O)[nH]c1=O]

with a compound of formula (II-0)

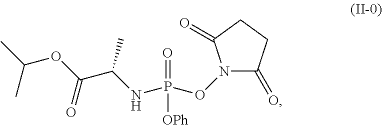 [SMILES: CC(C)OC(=O)[C@H](C)NP(=O)(Oc1ccccc1)ON1C(=O)CCC1=O]

to the compound of formula (I-1)

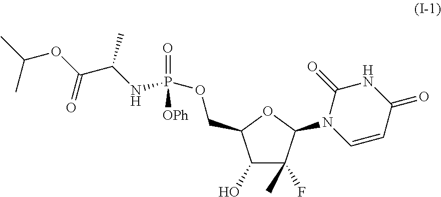 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

obtained from said reaction,
said compound of formula (II-0) comprising a compound of formula (II-a)

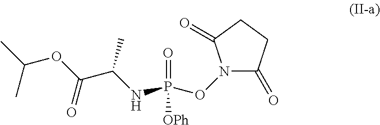 [SMILES: CC(C)OC(=O)[C@H](C)N[P@](=O)(Oc1ccccc1)ON1C(=O)CCC1=O]

and a compound of formula (II-b)

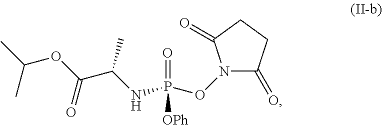 [SMILES: CC(C)OC(=O)[C@H](C)N[P@@](=O)(Oc1ccccc1)ON1C(=O)CCC1=O]

wherein the molar ratio of the compound of formula (II-a) relative to the compound of formula (II-b) is in the range of from 45:55 to 72:28, preferably in the range of from 45:55 to 60:40, more preferably in the range of from 45:55 to 55:45; wherein preferably, the molar ratio of the compound of formula (II-a) relative the compound of formula (II-b) is in the range of from 55:45 to 45:55, preferably in the range of from 54:46 to 46:54, more preferably in the range of from 53:47 to 47:53, more preferably in the range of from 52:48 to 48:52, more preferably in the range of from 51:49 to 49:51; wherein more preferably, the molar ratio of the compound of formula (II-a) relative the compound of formula (II-b) is 1:1 and wherein more preferably, the compound of formula (II-0) consists of the compound of formula (II-a) and the compound of formula (II-b);
wherein said method comprises employing a combination of a Lewis acid and a base as starting material in said reaction.

Further preferred mixtures, compounds, and uses are explicitly mentioned in the embodiment section hereinbelow.

### PROCESS ACCORDING TO THE INVENTION WITH CL AS THE LEAVING GROUP

The present invention further relates to a process for preparing of a compound of formula (I)

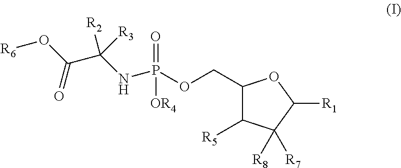

or a salt thereof, the process comprising
a′) reacting a compound of formula (II)

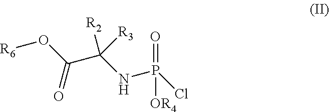

with a compound of formula (III)

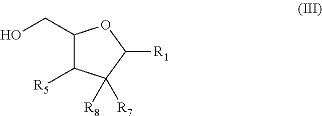

in the presence of a hydrogen chloride binding base, obtaining a mixture comprising the compound of formula (I).

Preferably, in particular in case the compound of formula (II) is

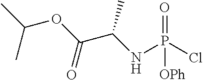 [SMILES: CC(C)OC(=O)[C@H](C)NP(=O)(Cl)Oc1ccccc1]

and the compound of formula (III) is

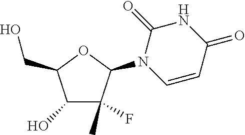 [SMILES: C[C@@]1(F)[C@H](O)[C@@H](CO)O[C@H]1n1ccc(=O)[nH]c1=O]

the hydrogen chloride binding base according to a′) is not, preferably does not comprise, N-methylimidazole with the proviso that if according to a′), a Lewis acid is used in combination with the hydrogen chloride binding base, the hydrogen chloride binding base may comprise or may be N-methylimidazole.

Preferably, the residue R₄ is phenyl, naphthyl, quinolinyl, isoquinolinyl, quinazolinyl or quinoxalinyl, each optionally substituted with at least one of C₁-C₆ alkyl, C₁-C₆ alkoxy, C₃-C₆ cycloalkyl, aryl, halogen, C(O)OH, CHO, C(O)(C₁-C₆ alkyl), C(O)(aryl), C(O)O(C₁-C₆ alkyl), C(O)ONH₂, C(O)ONH(C₁-C₆ alkyl) and CN. The term “C₁-C₆ alkyl” as used herein refers to alkyl residues having 1, 2, 3, 4, 5, or 6 carbon atoms. The term “C₁-C₆ alkoxy” as used herein refers to alkoxy residues having 1, 2, 3, 4, 5, or 6 carbon atoms. The term “C₃-C₆ cycloalkyl” as used herein refers to cycloalkyl residues wherein 3, 4, 5, or 6 carbon atoms constitute the ring structure.

Preferably, the residues R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, NO₂, C(O)OH, CHO, C(O)(C₁-C₆ alkyl), C(O)(aryl), C(O)O(C₁-C₆ alkyl), C(O)ONH₂, C(O)ONH(C₁-C₆ alkyl) and CN.

Preferably, the residue R₆ is C₁-C₆ alkyl or C₃-C₁₀ cycloalkyl optionally substituted with at least one of C₁-C₆ alkyl and aryl.

Preferably, R₁ is an optionally derivatized purinyl residue, including an adenine residue and a guanine residue, or an optionally derivatized pyrimidinyl residue, including a cytosine residue, a thymine residue and an uracil residue, linked to the furanose ring according to formula (III) through a carbon or nitrogen atom.

Preferably, R₇ and R₈ are independently H, OH, F, Cl, Br, I, azide, nitrile, NH₂, NHR₂₆, NR₂₆R₂₄, C(O)NH₂, C(O)NHR₂₆, C(O)NR₂₆R₂₄, C₁-C₆ alkyl optionally substituted with C₁-C₆ alkyl, or C₃-C₁₀ cycloalkyl optionally substituted with C₁-C₆ alkyl, wherein R₂₆ and R₂₄ are independently C₁-C₆ alkyl.

Preferably, R₉ is H, OH, C₁-C₆ alkoxy, OC(O)R₂₅, or C₁-C₆ alkyl optionally substituted with C₁-C₆ alkyl or aryl, wherein R₂₅ is C₁-C₆ alkyl or aryl.

Step a′)

The P atom is a chirality center of the compound of formula (II). It is preferred that according to a′), the compound of formula (II) comprises a compound of formula (II-1)

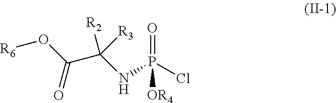

and a compound of formula (II-2)

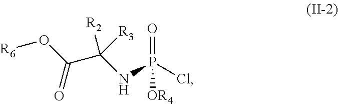

wherein the molar ratio of the compound of formula (II-1) relative the compound of formula (II-2) is in the range of from 55:45 to 45:55, more preferably in the range of from 54:46 to 46:54, more preferably in the range of from 53:47 to 47:53, more preferably in the range of from 52:48 to 48:52, more preferably in the range of from 51:49 to 49:51. More preferably, the molar ratio of the compound of formula (II-1) relative the compound of formula (II-2) is 1:1. More preferably, the compound of formula (II) consists of the compound of formula (II-1) and the compound of formula (II-2).

Preferably, from step a′) of the process of the present invention, a mixture is obtained which comprises the compound of formula (I) wherein the compound of formula (I) comprises a compound of formula (I-1)

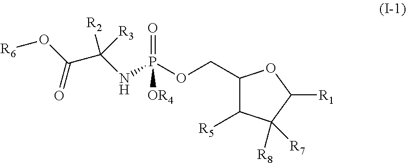

and a compound of formula (I-2)

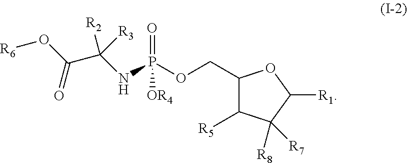

It is preferred that in the mixture obtained from a′), the molar ratio of the compound of formula (I-1) relative to the compound of formula (I-2) is at least 65:35, more preferably at least 70:30, more preferably at least 75:25, more preferably at least 80:20. It is conceivable that the molar ratio of the compound of formula (I-1) relative to the compound of formula (I-2) may be at least 85:15 or at least 90:10 or at least 95:5 or at least 99:1. More preferably, the compound of formula (I) comprised in the mixture obtained from a′) consists of the compound of formula (I-1) and the compound of formula (I-2).

Preferably, one of R₇ and R₈ may be C₁-C₆ alkyl, preferably methyl, and one of R₇ and R₈ may be F, Cl, OH, CN, or NH₂. Further preferably, R₁ may be an optionally derivatized pyrimidinyl residue, preferably a uracil residue. Also preferably, R₁ may be an optionally derivatized purinyl residue. Therefore, among others, the following compounds of formula (I) may be preferably prepared by the process of the present invention:

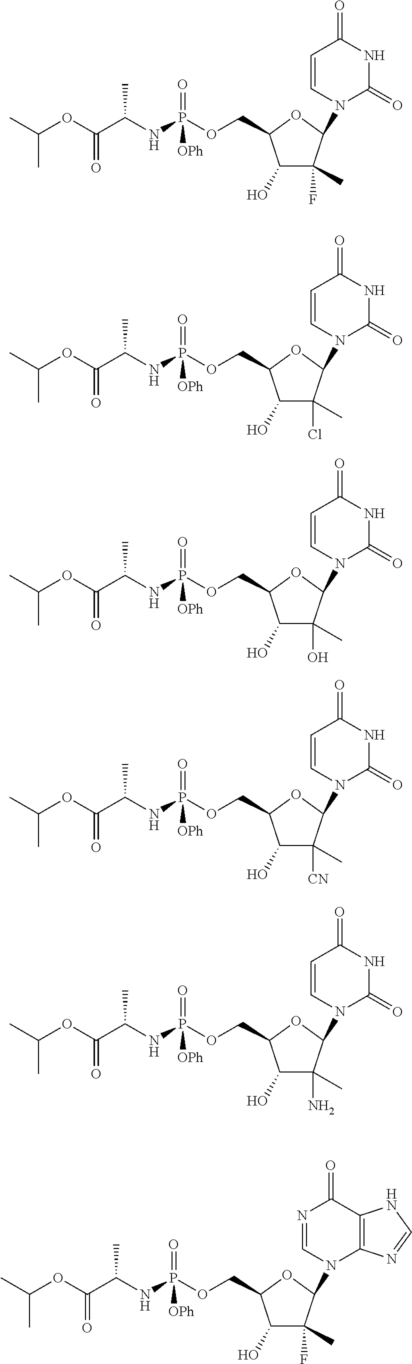

Preferably, regarding the compound of formula (III) employed in a′), the residues R₇ and R₈ are independently H or methyl.

More preferably, the compound of formula (III) is

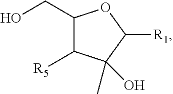

more preferably

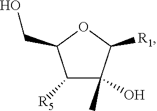

more preferably

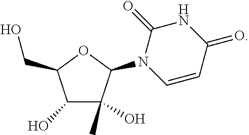 [SMILES: C[C@@]1(O)[C@H](O)[C@@H](CO)O[C@H]1n1ccc(=O)[nH]c1=O]

Preferably, the compound of formula (II-1) is a compound of formula

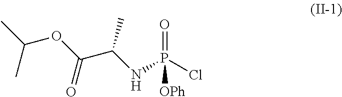 [SMILES: CC(C)OC(=O)[C@H](C)N[P@](=O)(Cl)Oc1ccccc1]

and the compound of formula (II-2) is a compound of formula

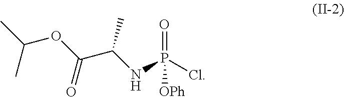 [SMILES: CC(C)OC(=O)[C@H](C)N[P@@](=O)(Cl)Oc1ccccc1]

Therefore, it is preferred that the compound of formula (I-1) is a compound

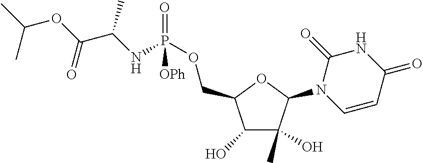 [SMILES: CC(C)OC(=O)[C@H](C)N[P@](=O)(OC[C@H]1O[C@@H](n2ccc(=O)[nH]c2=O)[C@](C)(O)[C@@H]1O)Oc1ccccc1]

and the compound of formula (I-2) is a compound

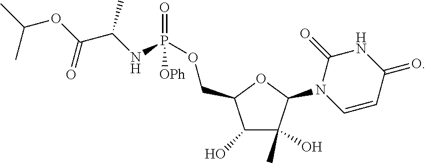 [SMILES: CC(C)OC(=O)[C@H](C)N[P@@](=O)(OC[C@H]1O[C@@H](n2ccc(=O)[nH]c2=O)[C@](C)(O)[C@@H]1O)Oc1ccccc1]

Even more preferably, the compound of formula (III) employed in a′) is a compound of formula

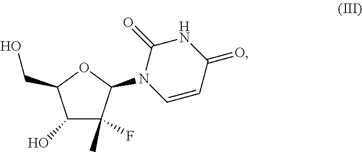 [SMILES: C[C@@]1(F)[C@H](O)[C@@H](CO)O[C@H]1n1ccc(=O)[nH]c1=O]

the compound of formula (II) is a compound of formula

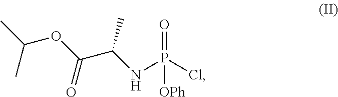 [SMILES: CC(C)OC(=O)[C@H](C)NP(=O)(Cl)Oc1ccccc1]

and the compound of formula (I) is a compound of formula

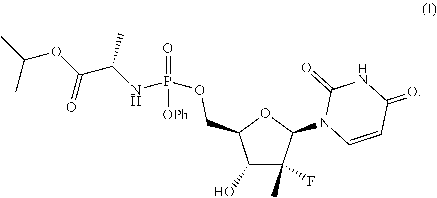 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

Therefore, the present invention relates to a process for preparing a compound of formula (I)

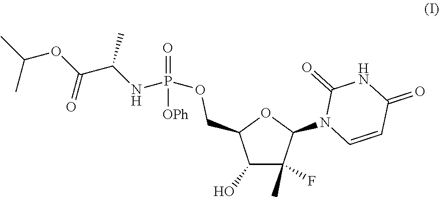 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a′) reacting a compound of formula (II)

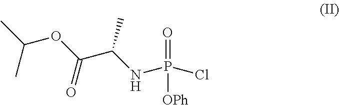 [SMILES: CC(C)OC(=O)[C@H](C)NP(=O)(Cl)Oc1ccccc1]

with a compound of formula (III)

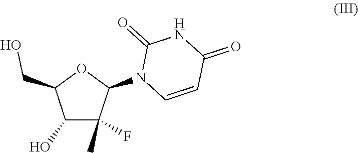 [SMILES: C[C@@]1(F)[C@H](O)[C@@H](CO)O[C@H]1n1ccc(=O)[nH]c1=O]

in the presence of a hydrogen chloride binding base which is not N-methylimidazole, obtaining a mixture comprising the compound of formula (I),
   wherein the compound of formula (II) comprises a compound of formula (II-1)

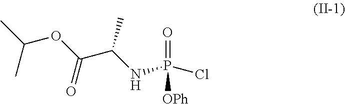 [SMILES: CC(C)OC(=O)[C@H](C)N[P@](=O)(Cl)Oc1ccccc1]

and a compound of formula (II-2)

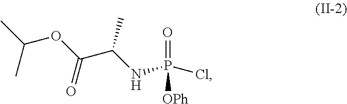 [SMILES: CC(C)OC(=O)[C@H](C)N[P@](=O)(Cl)Oc1ccccc1]

wherein the molar ratio of the compound of formula (II-1) relative the compound of formula (II-2) is in the range of from 55:45 to 45:55, with the proviso that if according to a, a Lewis acid is used in combination with the hydrogen chloride binding base, the hydrogen chloride binding base may comprise or may be N-methylimidazole.

The hydrogen chloride binding base employed in a′) is preferably an organic base, more preferably an organic nitrogenous base, more preferably a tertiary organic nitrogenous base. More preferably, the organic hydrogen chloride binding base comprises one or more of an amine, an amidine, and a heteroaromatic compound comprising a basic ring-nitrogen atom. More preferably, the organic hydrogen chloride binding base comprises one or more of ethyldiisopropylamine, triethylamine, diethylamine, 1,8-diazabicycloundec-7-ene, pyridine, quinoline, isoquinoline, acridine, pyrazine, imidazole, benzimidazole, and pyrazole. More preferably, the organic hydrogen chloride binding base consists of one or more of ethyldiisopropylamine, triethylamine, diethylamine, 1,8-diazabicycloundec-7-ene, pyridine, quinoline, isoquinoline, acridine, pyrazine, imidazole, benzimidazole, and pyrazole. More preferably, the hydrogen chloride binding base comprises triethylamine. More preferably, the hydrogen chloride binding base is triethylamine.

It is further contemplated that in the process as disclosed above, when step a′) is carried out in a solvent, the solvent is not an anhydrous solvents selected from dichloromethane, 2-methyl tetrahydrofuran, tetrahydrofuran, methyl-t-butyl ether, ethyl acetate, acetonitrile, cyclopentyl methylether, 1,4-dioxane, acetone, methyl ethyl ketone, methyl isobutyl ketone, diisopropyl ethyl amine, tripropylamine, tributylamine and their combinations or any functional equivalent thereof. In this embodiment the carrying out of step a′) preferably does not comprise the use of a Lewis acid.

In the process as disclosed above, it is further contemplated that the base is not selected from tripropyl amine, tributyl amine, diisopropyl ethyl amine, and their combinations, or any functional equivalent thereof. In this embodiment the carrying out of step a′) preferably does not comprise the use of a Lewis acid.

In the process as disclosed above, it is further contemplated that that the base selected from tripropylamine, tributylamine, diisopropylethylamine or any functional equivalent bases is not in combination with a solvent selected from dichloromethane, 2-methyl tetrahydrofuran, tetrahydrofuran, methyl-t-butyl ether, ethyl acetate, acetonitrile, cyclopentyl methylether, 1,4-dioxane, acetone, methyl ethyl ketone, methyl isobutyl ketone, diisopropyl ethyl amine, tripropylamine. In this embodiment the carrying out of step a′) preferably does not comprise the use of a Lewis acid.

In the process as disclosed above, it is further contemplated that that when the base is tripropylamine the solvent is not acetone or methyl-isobutyl-ketone or methyl-t-butyl ether or ethyl acetate or that when the base is diisopropylethylamine the solvent is not methyl-t-butyl ether or that when the base is THF the solvent is not tributylamine. In this embodiment the carrying out of step a′) preferably does not comprise the use of a Lewis acid.

Therefore, the present invention preferably relates to a process for preparing a compound of formula (I)

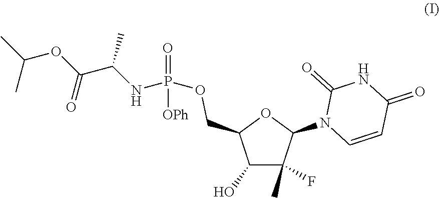 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a′) reacting a compound of formula (II)

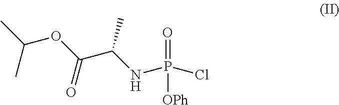 [SMILES: CC(C)OC(=O)[C@H](C)NP(=O)(Cl)Oc1ccccc1]

with a compound of formula (III)

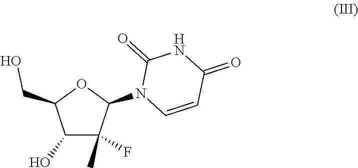 [SMILES: C[C@@]1(F)[C@H](O)[C@@H](CO)O[C@H]1n1ccc(=O)[nH]c1=O]

in the presence of a hydrogen chloride binding base comprising, preferably consisting of, one or more of ethyldiisopropylamine, triethylamine, diethylamine, 1,8-diazabicycloundec-7-ene, pyridine, quinoline, isoquinoline, acridine, pyrazine, imidazole, benzimidazole, and pyrazole, obtaining a mixture comprising the compound of formula (I),
   wherein the compound of formula (II) comprises a compound of formula (II-1)

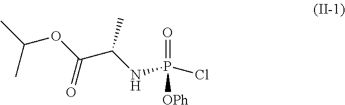 [SMILES: CC(C)OC(=O)[C@H](C)N[P@](=O)(Cl)Oc1ccccc1]

and a compound of formula (II-2)

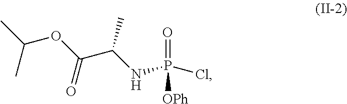 [SMILES: CC(C)OC(=O)[C@H](C)N[P@](=O)(Cl)Oc1ccccc1]

wherein the molar ratio of the compound of formula (II-1) relative the compound of formula (II-2) is in the range of from 55:45 to 45:55, preferably being 1:1.

Therefore, the present invention preferably relates to a process for preparing a compound of formula (I)

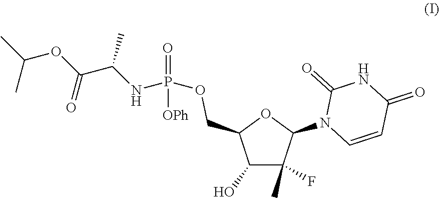 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a′) reacting a compound of formula (II)

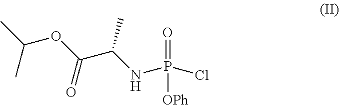 [SMILES: CC(C)OC(=O)[C@H](C)NP(=O)(Cl)Oc1ccccc1]

with a compound of formula (III)

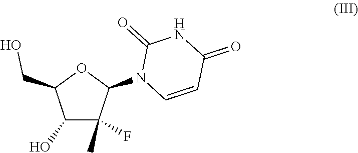 [SMILES: C[C@@]1(F)[C@H](O)[C@@H](CO)O[C@H]1n1ccc(=O)[nH]c1=O]

in the presence of a hydrogen chloride binding base comprising, preferably consisting of, triethylamine, obtaining a mixture comprising the compound of formula (I),
   wherein the compound of formula (II) comprises a compound of formula (II-1)

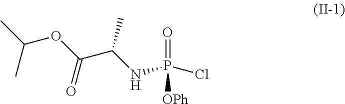 [SMILES: CC(C)OC(=O)[C@H](C)N[P@](=O)(Cl)Oc1ccccc1]

and a compound of formula (II-2)

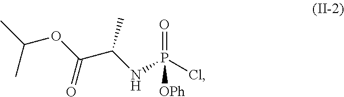 [SMILES: CC(C)OC(=O)[C@H](C)N[P@](=O)(Cl)Oc1ccccc1]

wherein the molar ratio of the compound of formula (II-1) relative the compound of formula (II-2) is in the range of from 55:45 to 45:55, preferably being 1:1.

Preferably, the molar ratio of the compound of formula (II-1) relative the compound of formula (II-2) is in the range of from 54:46 to 46:54, more preferably in the range of from 53:47 to 47:53, more preferably in the range of from 52:48 to 48:52, more preferably in the range of from 51:49 to 49:51. More preferably, the molar ratio of the compound of formula (II-1) relative the compound of formula (II-2) is 1:1. It is preferred that the compound of formula (II) employed in (a′) consists of the compound of formula (II-1) and the compound of formula (II-2).

Therefore, the present invention relates to the process as defined above, wherein the compound of formula (II) employed in a) consists of the compound of formula (II-1) and the compound of formula (II-2), wherein the molar ratio of the compound of formula (II-1) relative the compound of formula (II-2) is 1:1.

Regarding possible methods to prepare the compound of formula (II), reference is made, for example, to WO 2008/121634 A, Example 25, the respective content of which document is included herein by reference. Regarding possible methods to prepare the compound of formula (III), reference is made, for example, to WO 2008/121634 A, Example 4, the respective content of which document is included herein by reference.

As mentioned above, the process of the present invention is characterized by an advantageous diastereoselectivity to the valuable product, the compound of formula (I-1). Thus, it is preferred that in the mixture obtained from a′), the compound of formula (I) comprises a compound of formula (I-1)

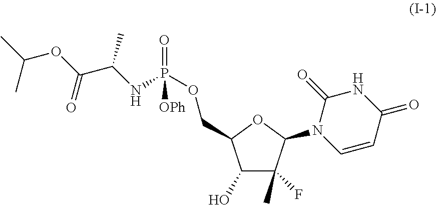 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and a compound of formula (I-2)

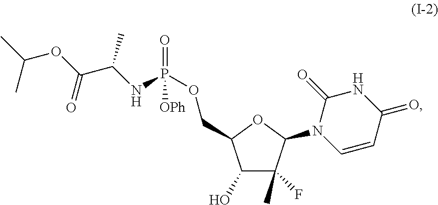 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

wherein the molar ratio of the compound of formula (I-1) relative to the compound of formula (I-2) is greater than 55:45, preferably at least 60:40. More preferably, said molar ratio is at least 65:35, more preferably at least 70:30, more preferably at least 75:25, more preferably at least 80:20. It is conceivable that the molar ratio of the compound of formula (I-1) relative to the compound of formula (I-2) may be at least 85:15 or at least 90:10 or at least 95:5 or at least 99:1. More preferably, the compound of formula (I) comprised in the mixture obtained from a′) consists of the compound of formula (I-1) and the compound of formula (I-2).

Therefore, the present invention relates to the process as defined above, wherein the compound of formula (II) employed in a′) consists of the compound of formula (II-1) and the compound of formula (II-2), wherein the molar ratio of the compound of formula (II-1) relative the compound of formula (II-2) is 1:1, wherein the compound of formula (I) comprised in the mixture obtained from a′) consists of the compound of formula (I-1) and the compound of formula (I-2), and wherein the molar ratio of the compound of formula (I-1) relative to the compound of formula (I-2) is at least 65:35, preferably at least 80:20.

Regarding the amount of the hydrogen chloride binding base relative to the amount of the compound of formula (III) employed in a′), no specific restrictions exist. Preferably, prior to the reaction according to a′), the molar ratio of the hydrogen chloride binding base relative to the compound of formula (III) is in the range of from 0.1:1 to 5:1. More preferably, prior to the reaction according to a′), the molar ratio of the hydrogen chloride binding base relative to the compound of formula (III) is in the range of from 0.5:1 to 5:1, more preferably in the range of from 1:1 to 5:1, more preferably in the range of from 2:1 to 5:1. More preferably, prior to the reaction according to a′), the molar ratio of the hydrogen chloride binding base relative to the compound of formula (III) is in the range of from 2:1 to 4:1, more preferably in the range of from 2.5:1 to 4.5:1, more preferably in the range of from 2.5:1 to 4:1, more preferably in the range of from 2.5:1 to 3.5:1.

Therefore, the present invention preferably relates to a process for preparing a compound of formula (I)

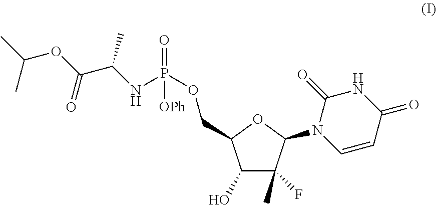 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a′) reacting a compound of formula (II)

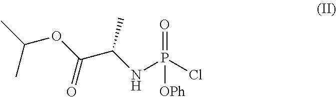 [SMILES: CC(C)OC(=O)[C@H](C)NP(=O)(Cl)Oc1ccccc1]

with a compound of formula (III)

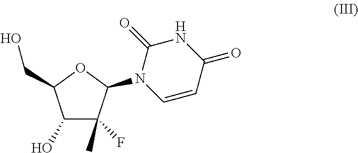 [SMILES: C[C@@]1(F)[C@H](O)[C@@H](CO)O[C@H]1n1ccc(=O)[nH]c1=O]

in the presence of a hydrogen chloride binding base comprising, preferably consisting of, one or more of ethyldiisopropylamine, triethylamine, diethylamine, 1,8-diazabicycloundec-7-ene, pyridine, quinoline, isoquinoline, acridine, pyrazine, imidazole, benzimidazole, and pyrazole, said hydrogen chloride binding base more preferably comprising, more preferably consisting of, trimethylamine, obtaining a mixture comprising the compound of formula (I),
   wherein the compound of formula (II) comprises a compound of formula (II-1)

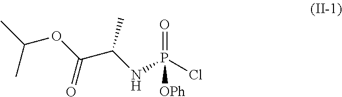 [SMILES: CC(C)OC(=O)[C@H](C)N[P@](=O)(Cl)Oc1ccccc1]

and a compound of formula (II-2)

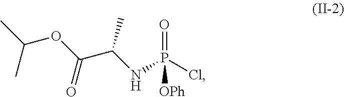 [SMILES: CC(C)OC(=O)[C@H](C)N[P@](=O)(Cl)Oc1ccccc1]

wherein the molar ratio of the compound of formula (II-1) relative the compound of formula (II-2) is in the range of from 55:45 to 45:55, preferably being 1:1, and wherein prior to the reaction according to a′), the molar ratio of the hydrogen chloride binding base relative to the compound of formula (III) is in the range of from 0.5:1 to 5:1, preferably in the range of from 2.5:1 to 3.5:1.

Further according to the present invention, it was found that the diastereoselectivity to the compound of formula (I-1), in particular the compound

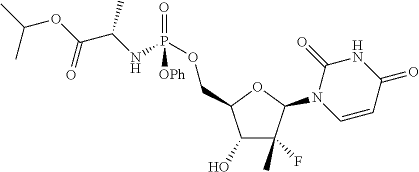 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

can be even more improved if the compound of formula (III) is reacted with the compound of formula (II) in the presence of the hydrogen chloride binding base and a Lewis acid.

No specific restrictions exist with regard to the chemical nature of the Lewis acid employed in a′). Preferably, the Lewis acid comprises a twice positively charged ion or a three times positively charged ion, more preferably a twice positively charged metal ion or a three times positively charged metal ion. Generally, it is also conceivable that the Lewis acid comprises a twice positively charged ion and a three times positively charged ion, preferably a twice positively charged metal ion and a three times positively charged metal ion. With regard to the twice positively charged ion, it is preferred that it comprises, more preferably is, a Zn ion, a Mg ion, a Cu ion, or an Fe ion. More preferably, the twice positively charged ion comprises, more preferably is, a Zn ion or a Mg ion. More preferably, the twice positively charged ion comprises, more preferably is, a Zn ion. With regard to the three times positively charged ion, it is preferred that it comprises, more preferably is, a Mn ion.

Regarding the Lewis acid comprising a twice positively charged ion comprising, more preferably being, a Zn ion, no specific restrictions exist. Preferred Lewis acids comprise, more preferably are, Zn halides. More preferably, the Lewis acid comprises, preferably is, one or more of ZnBr₂, ZnCl₂, and ZnI₂. More preferably, the Lewis acid comprises, preferably is, ZnBr₂.

It is also conceivable that the Lewis acid is one or more of ZnBr₂, ZnCl₂, ZnI₂, MgBr₂, MgBr₂.OEt₂, CuCl₂, Cu(acetylacetonate)₂, and Fe(II) fumarate.

Regarding the Lewis acid comprising a three times positively charged ion comprising, more preferably being, a Mn ion, no specific restrictions exist. Preferred Lewis acids comprise, more preferably are, Mn(acetylacetonate)₃.

Regarding the amount of the Lewis acid relative to the amount of the compound of formula (III) employed in a′), no specific restrictions exist. Preferably, prior to the reaction according to a′), the molar ratio of the Lewis acid relative to the compound of formula (III) is in the range of from 0.1:1 to 5:1. More preferably, prior to the reaction according to a′), the molar ratio of the Lewis acid relative to the compound of formula (III) is in the range of from 0.2:1 to 5:1, preferably in the range of from 0.5:1 to 3:1, more preferably in the range of from 0.75:1 to 1.5:1, more preferably in the range of from 0.75:1 to 1.25:1.

Thus, the present invention preferably relates to a process for preparing a compound of formula (I)

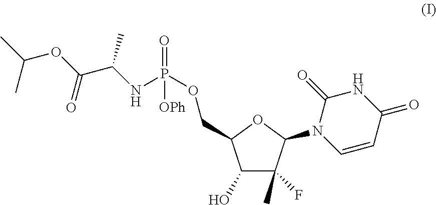 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a′) reacting a compound of formula (II)

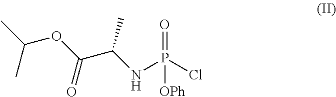 [SMILES: CC(C)OC(=O)[C@H](C)NP(=O)(Cl)Oc1ccccc1]

with a compound of formula (III)

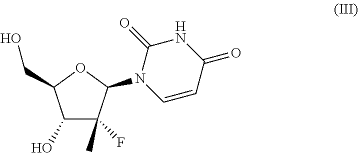 [SMILES: C[C@@]1(F)[C@H](O)[C@@H](CO)O[C@H]1n1ccc(=O)[nH]c1=O]

in the presence of a Lewis acid preferably comprising a Zn ion, more preferably comprising, more preferably being, ZnBr₂, and in the presence of a hydrogen chloride binding base comprising, preferably consisting of, one or more of ethyldiisopropylamine, triethylamine, diethylamine, 1,8-diazabicycloundec-7-ene, pyridine, quinoline, isoquinoline, acridine, pyrazine, imidazole, benzimidazole, and pyrazole, said hydrogen chloride binding base more preferably comprising, more preferably consisting of, trimethylamine, obtaining a mixture comprising the compound of formula (I),
   wherein the compound of formula (II) comprises a compound of formula (II-1)

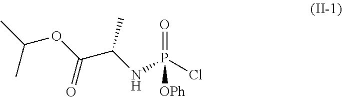 [SMILES: CC(C)OC(=O)[C@H](C)N[P@](=O)(Cl)Oc1ccccc1]

and a compound of formula (II-2)

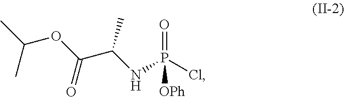 [SMILES: CC(C)OC(=O)[C@H](C)N[P@](=O)(Cl)Oc1ccccc1]

wherein the molar ratio of the compound of formula (II-1) relative the compound of formula (II-2) is in the range of from 55:45 to 45:55, preferably being 1:1, wherein prior to the reaction according to a′), the molar ratio of the hydrogen chloride binding base relative to the compound of formula (III) is preferably in the range of from 0.5:1 to 5:1, more preferably in the range of from 2.5:1 to 3.5:1, and wherein prior to the reaction according to a′), the molar ratio of the Lewis acid relative to the compound of formula (III) is preferably in the range of from 0.1:1 to 5:1, more preferably in the range of from 0.75:1 to 1.5:1.

Regarding the amount of the compound of formula (II) employed in a′) relative to the amount of the compound of formula (III) employed in a′), no specific restrictions exist. Preferably, prior to the reaction according to a′), the molar ratio of the compound of formula (II) relative to the compound of formula (III) is in the range of from 0.5:1 to 5:1. More preferably, prior to the reaction according to a′), the molar ratio of the compound of formula (II) relative to the compound of formula (III) is in the range of from 0.6:1 to 4:1, more preferably in the range of from 0.7:1 to 3:1. More preferably, prior to the reaction according to a′), the molar ratio of the compound of formula (II) relative to the compound of formula (III) is in the range of from 0.8:1 to 2:1, more preferably in the range of from 0.9:1 to 1.2:1.

Thus, the present invention preferably relates to a process for preparing a compound of formula (I)

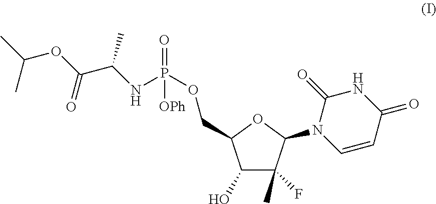 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a′) reacting a compound of formula (II)

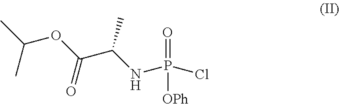 [SMILES: CC(C)OC(=O)[C@H](C)NP(=O)(Cl)Oc1ccccc1]

with a compound of formula (III)

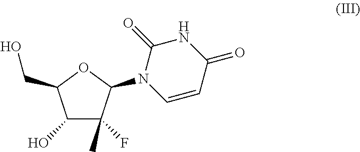 [SMILES: C[C@@]1(F)[C@H](O)[C@@H](CO)O[C@H]1n1ccc(=O)[nH]c1=O]

in the presence of a Lewis acid preferably comprising a Zn ion, more preferably comprising, more preferably being, ZnBr₂, and in the presence of a hydrogen chloride binding base comprising, preferably consisting of, one or more of ethyldiisopropylamine, triethylamine, diethylamine, 1,8-diazabicycloundec-7-ene, pyridine, quinoline, isoquinoline, acridine, pyrazine, imidazole, benzimidazole, and pyrazole, said hydrogen chloride binding base more preferably comprising, more preferably consisting of, trimethylamine, obtaining a mixture comprising the compound of formula (I),
   wherein the compound of formula (II) comprises a compound of formula (II-1)

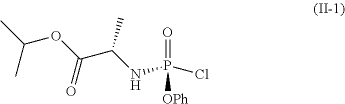 [SMILES: CC(C)OC(=O)[C@H](C)N[P@](=O)(Cl)Oc1ccccc1]

and a compound of formula (II-2)

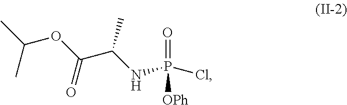 [SMILES: CC(C)OC(=O)[C@H](C)N[P@](=O)(Cl)Oc1ccccc1]

wherein the molar ratio of the compound of formula (II-1) relative the compound of formula (II-2) is in the range of from 55:45 to 45:55, preferably being 1:1, wherein prior to the reaction according to a′), the molar ratio of the hydrogen chloride binding base relative to the compound of formula (III) is preferably in the range of from 0.5:1 to 5:1, more preferably in the range of from 2.5:1 to 3.5:1, wherein prior to the reaction according to a′), the molar ratio of the Lewis acid relative to the compound of formula (III) is preferably in the range of from 0.1:1 to 5:1, more preferably in the range of from 0.75:1 to 1.5:1, and wherein prior to the reaction according to a′), the molar ratio of the compound of formula (II) relative to the compound of formula (III) is preferably in the range of from 0.5:1 to 5:1, more preferably in the range of from 0.9:1 to 1.2:1.

Generally, it may be conceivable that the reacting according to a′) is carried in the absence of an additional solvent. According to the present invention, it is preferred that according to a′), the compound of formula (II) is reacted with the compound of formula (III) in the presence of the hydrogen chloride binding base, preferably in the presence of the Lewis acid, and in the presence of a solvent.

Preferably, the solvent comprises, preferably is, one or more organic solvents, preferably one or more aprotic organic solvents. Generally, every aprotic organic solvent can be employed which allows to carry out the reacting according to a′). Preferably, the aprotic organic solvent Comprises, more preferably consists of, one or more of methylene chloride, methyl tert-butyl ether, tetrahydrofuran, dimethylsulphoxide, and dimethylformamide. More preferably, solvent comprises, preferably is, tetrahydrofuran.

Thus, the present invention preferably relates to a process for preparing a compound of formula (I)

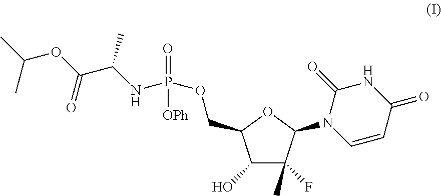 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, wherein the process comprises
a′) reacting a compound of formula (II)

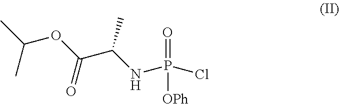 [SMILES: CC(C)OC(=O)[C@H](C)NP(=O)(Cl)Oc1ccccc1]

with a compound of formula (III)

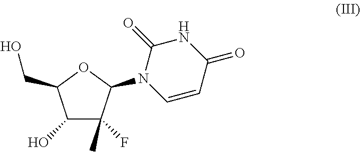 [SMILES: C[C@@]1(F)[C@H](O)[C@@H](CO)O[C@H]1n1ccc(=O)[nH]c1=O]

in the presence of a solvent, preferably tetrahydrofuran, of a Lewis acid being ZnBr₂ and of a hydrogen chloride binding base being trimethylamine, obtaining a mixture comprising the compound of formula (I),
   wherein the compound of formula (II) comprises a compound of formula (II-1)

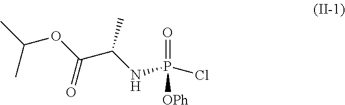 [SMILES: CC(C)OC(=O)[C@H](C)N[P@](=O)(Cl)Oc1ccccc1]

and a compound of formula (II-2)

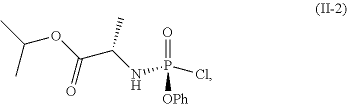 [SMILES: CC(C)OC(=O)[C@H](C)N[P@](=O)(Cl)Oc1ccccc1]

wherein the molar ratio of the compound of formula (II-1) relative the compound of formula (II-2) is 1:1, wherein prior to the reaction according to a′), the molar ratio of the hydrogen chloride binding base relative to the compound of formula (III) is in the range of from 2.5:1 to 3.5:1, the molar ratio of the Lewis acid relative to the compound of formula (III) is in the range of from 0.75:1 to 1.5:1, and the molar ratio of the compound of formula (II) relative to the compound of formula (III) is in the range of from 0.9:1 to 1.2:1.

The temperature at which the reacting according to a′) is carried out can be suitably chosen, depending on the chemical nature of components of the mixture which is subjected to reaction conditions according to a′), and in particular, if present, the chemical nature of the solvent. Preferably, the reacting according to a′) is carried out at a temperature in the range of from 0 to 80° C., more preferably in the range of from 0 to 70° C., more preferably in the range of from 0 to 60° C., more preferably in the range of from 0 to 50° C., more preferably in the range of from 0 to 50° C., more preferably in the range of from 0 to 40° C., more preferably in the range of from 0 to 30° C., more preferably in the range of from 0 to 25° C. More preferably, the reacting according to a′) is carried out at a temperature in the range of from 0 to 20° C., more preferably in the range of from 0 to 15° C., more preferably in the range of from 0 to 10° C., more preferably in the range of from 0 to 5° C.

In particular in case the reaction according to a′) is carried out in the presence of the hydrogen chloride binding base without a Lewis acid, it may be preferred to carry out the reacting at two or more temperatures, preferably at two temperatures. In this case, it is preferred that in a first reacting stage, the reacting is carried out at a temperature in the range of from 0 to 10° C., preferably in the range of from 0 to 5° C., and that in a subsequent second reacting stage, the reacting is carried out at a temperature in the range of from 15 to 40° C., preferably in the range of from 20 to 30° C.

Generally, the compounds subjected to reacting in a′) can be admixed in any sequence. Preferably, in case no Lewis acid is employed, the compound of formula (II) is admixed with the compound of formula (III) wherein, if a solvent is used, the compound of formula (II) can be preferably employed dissolved in this solvent; it is further preferred that the resulting mixture is then cooled to a temperature in the range of from 0 to 15° C., more preferably in the range of from 0 to 10° C., more preferably in the range of from 0 to 5° C. and to the thus cooled mixture, the hydrogen chloride binding base is added. Preferably, in case a Lewis acid is employed, the compound of formula (II) is admixed with the compound of formula (III) wherein, if a solvent is used, the compound of formula (II) can be preferably employed dissolved in this solvent; it is further preferred that to the resulting mixture, the Lewis acid is added; it is further preferred that the resulting mixture is then cooled to a temperature in the range of from 0 to 15° C., more preferably in the range of from 0 to 10° C., more preferably in the range of from 0 to 5° C. and to the thus cooled mixture, the hydrogen chloride binding base is added.

The period of time for which the reacting according to a′) is carried out can be suitably chosen. Preferably, the reacting according to a′) is carried out for a period of time in the range of from 0.5 to 48 h, more preferably in the range of from 0.75 to 42 h, more preferably in the range of from 1 to 36 h. More preferably, the reacting according to a′) is carried out for a period of time in the range of from 1.5 to 20 h, more preferably in the range of from 2 to 24 h. Preferred ranges are from 2 to 6 h or from 6 to 10 h or from 10 to 14 h or from 14 to 19 h or from 19 to 24 h.

Preferably, during reacting according to a′), the reaction mixture is agitated, more preferably mechanically agitated, more preferably stirred. The term “agitation” as used herein relates to any motion of a macroscopic constituent of the reaction mixture which is induced from outside, relative to another macroscopic constituent of the reaction mixture. The term “mechanical agitation” as used herein relates to any motion of a macroscopic constituent of the reaction mixture which is induced from outside via a device, such as shaking or stirring or sonication, relative to another macroscopic constituent of the reaction mixture. The term “stirring” as used herein relates to any motion of a macroscopic constituent of the reaction mixture which is induced from outside via a stirring device, relative to another macroscopic constituent of the reaction mixture.

Step b′)

Preferably, the compound of formula (I) comprised in the mixture obtained from a′) is suitably separated from said mixture. Generally, it is preferred that from said separating, a composition is obtained which comprises the compound of formula (I), in particular a composition comprising the compound of formula (I) comprising the compound of formula (I-1) and the compound of formula (I-2), preferably a composition comprising the compound of formula (I) consisting of the compound of formula (I-1) and the compound of formula (I-2). Therefore, the present invention also relates to the process as defined above, further comprising b′) separating the compound of formula (I) from the mixture obtained in a′).

It is preferred that the compound of formula (I) is separated from the liquid phase of the mixture obtained in a′) wherein the separating preferably includes filtration or centrifugation, more preferably filtration. Further, it is preferred that the compound of formula (I) obtained from filtration or centrifugation, preferably filtration, is washed and/or dried, preferably washed and dried. No specific limitations exist regarding the chemical nature of the washing agent. Preferred washing agents include isopropyl acetate. No specific limitations exist for the drying conditions. Preferred drying conditions include a pressure below 1 bar, preferably drying in vacuo. Further, it is preferred that the compound of formula (I), preferably after drying, is further dissolved in one or more solvents, including, for example, toluene and/or isopropyl acetate.

The thus dissolved compound of formula (I) can be further subjected to extraction, including, for example, extraction with aqueous sodium chloride, obtaining an organic phase from which the solvent is preferably removed whereafter the solid compound of formula (I) is preferably dissolved in one or more further solvents. If extraction is carried, it is, for example, preferred to dissolve the compound of formula (I), after separation from the liquid phase of the mixture obtained in a′) and drying, in a first organic solvent, for example, isopropyl acetate, subject the thus obtained solution to extraction, for example with aqueous sodium chloride, obtaining an organic phase from which the solvent is suitably removed, and dissolve the thus obtained solid compound of formula (I) in a second organic solvent, for example toluene.

Therefore, the present invention also relates to the process as defined above, preferably further comprising
b′) separating the compound of formula (I) from the mixture obtained in a′), obtaining the compound of formula (I) dissolved in a solvent, preferably an organic solvent.

### Further Steps

It is preferred that in the mixture obtained from b′) preferably comprising the compound of formula (I) dissolved in a solvent, preferably an organic solvent, the compound of formula (I-1) is suitably crystallized. From this crystallization, the compound of formula (I-1) is obtained in its mother liquor from which it is preferably suitably separated.

Therefore, the present invention also relates to the process as defined above, further comprising
c′) crystallizing the compound of formula (I-1), preferably from the mixture obtained from b′) comprising the compound of formula (I) dissolved in a solvent.

Further, the present invention also relates to the process as defined above, further comprising
c′) crystallizing the compound of formula (I-1), preferably from the mixture obtained from b) comprising the compound of formula (I) dissolved in a solvent, obtaining the crystallized compound of formula (I-1) in its mother liquor;
d′) separating the crystallized compound of formula (I-1) from its mother liquor, obtaining the compound of formula (I-1) in crystalline form.

During crystallization in c′), it can be preferred that suitable seed crystals are added, preferably seed crystals of the compound of formula (I-1).

After the crystallization in c′) from which the crystallized compound of formula (I-1) is obtained in its mother liquor, the crystallized compound of formula (I-1) is preferably suitably separated from its mother liquor in d′), for example by filtration or centrifugation. The thus separated crystallized compound of formula (I-1) can be subjected to washing, wherein preferred washing agents include methyl tert-butyl ether, dichloromethane and mixtures thereof, and subject the optionally washed crystallized compound of formula (I-1) to drying. Preferred drying conditions include temperatures in the range of from 10 to 60° C., preferably in the range of from 30 to 50° C., and a pressure below ambient pressure.

Therefore, the present invention also relates to the process as defined above, further comprising
c′) crystallizing the compound of formula (I-1), preferably from the mixture obtained from b′) comprising the compound of formula (I) dissolved in a solvent, obtaining the crystallized compound of formula (I-1) in its mother liquor;
d′) separating the crystallized compound of formula (I) from its mother liquor, obtaining the compound of formula (I) in crystalline form, said separating comprising
   d1′) subjecting the mother liquor comprising the crystallized compound of formula (I-1) to filtration, obtaining a filter cake comprising the compound of formula (I-1);
   d2′) optionally washing the filter cake;
   d3′) drying the optionally washed filter cake, obtaining the compound of formula (I-1).

### Mixtures and Uses

Generally, the present invention also relates to a mixture which is obtainable or obtained by the process of the present invention, preferably obtainable or obtained from step a′) of a process of the present invention. More preferably, the present invention relates to a mixture which is obtainable or obtained from step a′) of a process as defined above, step a′) comprising reacting a compound of formula (II)

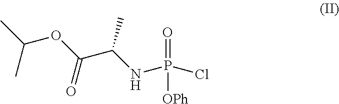 [SMILES: CC(C)OC(=O)[C@H](C)NP(=O)(Cl)Oc1ccccc1]

with a compound of formula (III)

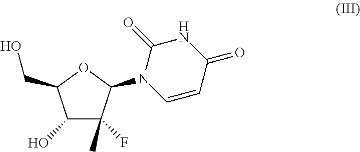 [SMILES: C[C@@]1(F)[C@H](O)[C@@H](CO)O[C@H]1n1ccc(=O)[nH]c1=O]

wherein the compound of formula (II) comprises a compound of formula (II-1)

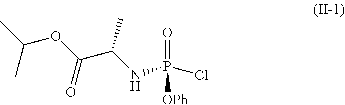 [SMILES: CC(C)OC(=O)[C@H](C)N[P@](=O)(Cl)Oc1ccccc1]

and a compound of formula (II-2)

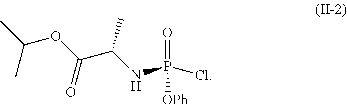 [SMILES: CC(C)OC(=O)[C@H](C)N[P@@](=O)(Cl)Oc1ccccc1]

Yet further, the present invention relates to a mixture which may be obtainable or obtained from step s) of the process as defined above, wherein said mixture comprises the compound of formula (I) comprising a compound of formula (I-1)

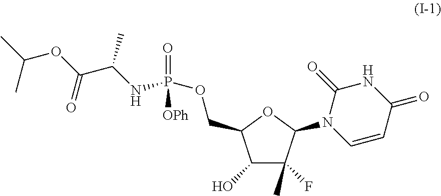 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and a compound of formula (I-2)

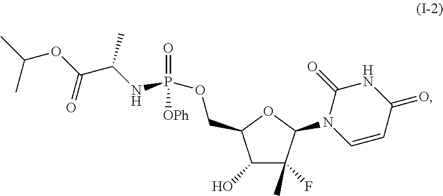 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

wherein the mixture, the molar ratio of the compound of formula (I-1) relative to the compound of formula (I-2) is at least 65:35, preferably at least 70:30, more preferably at least 75:25, more preferably at least 80:20, said mixture further comprising a hydrogen chloride binding base, which is not N-methylimidazole, with bound hydrogen chloride. Regarding further preferred embodiments of said mixture, reference is made to the disclosure above, in particular regarding preferred molar ratios disclosed above and preferred hydrogen chloride bases and, preferably, preferred Lewis bases. Further reference is made to the respective disclosure in the embodiment section of the present application. A preferred mixture of the present invention comprises the compound of formula (I) comprising a compound of formula (I-1)

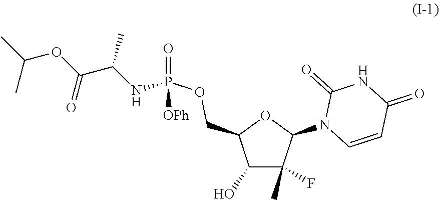 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and a compound of formula (I-2)

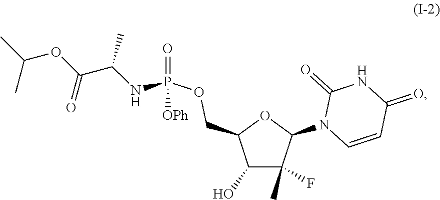 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

wherein in the mixture, the molar ratio of the compound of formula (I-1) relative to the compound of formula (I-2) is at least 80:20, said mixture further comprising a hydrogen chloride binding base which is triethylamine with bound hydrogen chloride, a Lewis base which is ZnBr₂, and preferably a solvent which is preferably tetrahydrofuran.

Preferably, this mixture is used for obtaining the compound of formula

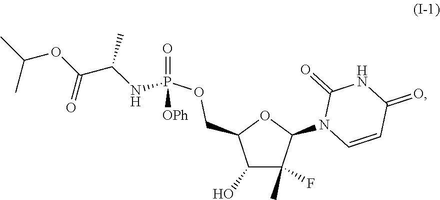 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

wherein obtaining the compound of formula (I-1) preferably comprises separating the compound of formula (I) from the mixture and crystallizing the compound of formula (I-1), obtaining the crystallized compound of formula (I-1) in its mother liquor and separating the crystallized compound of formula (I-1) from its mother liquor.

As mentioned above, the process of the present invention allows the diastereoselective preparation of the compound of formula (I-1) based on a phosphoric acid chloride. This reaction is based on a specific starting mixture which is subjected to reaction conditions. Therefore, the present invention also relates to this novel mixture which comprises a compound of formula (II)

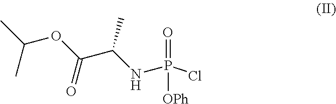 [SMILES: CC(C)OC(=O)[C@H](C)NP(=O)(Cl)Oc1ccccc1]

and a compound of formula (III)

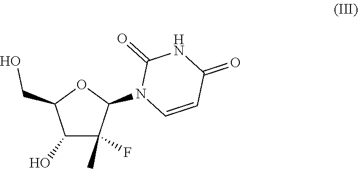 [SMILES: C[C@@]1(F)[C@H](O)[C@@H](CO)O[C@H]1n1ccc(=O)[nH]c1=O]

and a hydrogen chloride binding base which is not N-methylimidazole, wherein the compound of formula (II) comprises a compound of formula (II-1)

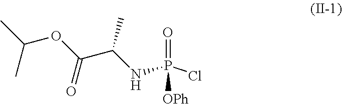 [SMILES: CC(C)OC(=O)[C@H](C)N[P@](=O)(Cl)Oc1ccccc1]

and a compound of formula (II-2)

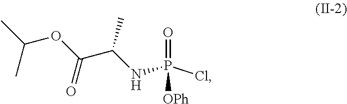 [SMILES: CC(C)OC(=O)[C@H](C)N[P@](=O)(Cl)Oc1ccccc1]

wherein the molar ratio of the compound of formula (II-1) relative the compound of formula (II-2) is in the range of from 55:45 to 45:55, preferably in the range of from 52:48 to 48:52, more preferably in the range of from 51:49 to 49:51, wherein more preferably, the molar ratio of the compound of formula (II-1) relative the compound of formula (II-2) is 1:1.

Regarding further preferred embodiments of said mixture, reference is made to the disclosure above, in particular regarding preferred molar ratios disclosed above and preferred hydrogen chloride bases and, preferably, preferred Lewis bases. Further reference is made to the respective disclosure in the embodiment section of the present application. A preferred mixture of the present invention comprises a compound of formula (II)

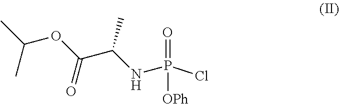 [SMILES: CC(C)OC(=O)[C@H](C)NP(=O)(Cl)Oc1ccccc1]

and a compound of formula (III)

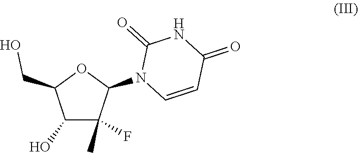 [SMILES: C[C@@]1(F)[C@H](O)[C@@H](CO)O[C@H]1n1ccc(=O)[nH]c1=O]

and a hydrogen chloride binding base which is triethylamine and a Lewis base which is ZnBr₂, wherein the compound of formula (II) comprises a compound of formula (II-1)

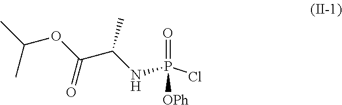 [SMILES: CC(C)OC(=O)[C@H](C)N[P@](=O)(Cl)Oc1ccccc1]

and a compound of formula (II-2)

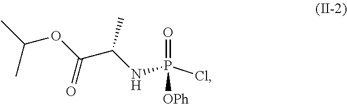 [SMILES: CC(C)OC(=O)[C@H](C)N[P@](=O)(Cl)Oc1ccccc1]

wherein the molar ratio of the compound of formula (II-1) relative the compound of formula (II-2) is 1:1, the molar ratio of the hydrogen chloride binding base relative to the compound of formula (III) is in the range of from 2.5:1 to 3.5:1, the molar ratio of the compound of formula (II) relative to the compound of formula (III) is in the range of from 0.9:1 to 1.2:1, and the molar ratio of the Lewis acid relative to the compound of formula (III) is in the range of from 0.75 to 1.5:1, the mixture preferably comprising a solvent, more preferably tetrahydrofuran.

Preferably, this mixture is used for obtaining the compound of formula

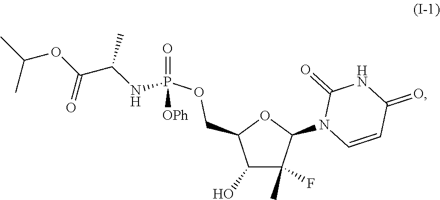 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

wherein obtaining the compound of formula (I-1) preferably comprises subjecting the mixture to reaction conditions obtaining a mixture comprising the compound of formula (I), separating the compound of formula (I) from the mixture and crystallizing the compound of formula (I-1), obtaining the crystallized compound of formula (I-1) in its mother liquor and separating the crystallized compound of formula (I-1) from its mother liquor.

As also mentioned above, the use of this mixture allows improving the diastereoselectivity to the compound of formula (I-1) when using the phosphoric acid chloride as starting material. Thus, the present invention also relates to the use of this mixture for improving the selectivity to the compound of formula (I-1)

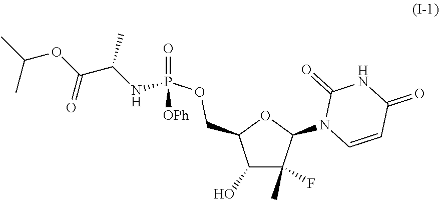 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

of the reaction of a compound of a compound of formula (III) with a compound of formula (II) comprising a compound of formula (II-1) and a compound of formula (II-2) wherein the molar ratio of the compound of formula (II-1) relative the compound of formula (II-2) is in the range of from 55:45 to 45:55, preferably in the range of from 52:48 to 48:52, more preferably in the range of from 51:49 to 49:51, wherein more preferably, the molar ratio of the compound of formula (II-1) relative the compound of formula (II-2) is 1:1. Further, the present invention relates to a method for improving the selectivity to the compound of formula (I-1)

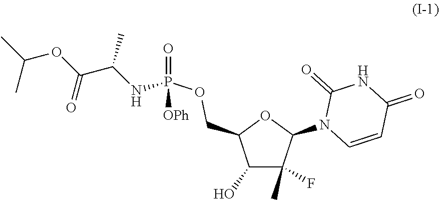 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

of the reaction of a compound of a compound of formula (III) with a compound of formula (II) comprising a compound of formula (II-1) and a compound of formula (II-2) wherein the molar ratio of the compound of formula (II-1) relative the compound of formula (II-2) is in the range of from 55:45 to 45:55, preferably in the range of from 52:48 to 48:52, more preferably in the range of from 51:49 to 49:51, wherein more preferably, the molar ratio of the compound of formula (II-1) relative the compound of formula (II-2) is 1:1, wherein said method comprises employing the above-mentioned mixture as starting material in said reaction.

Generally, it was found that in particular the combination of a Lewis acid and a hydrogen chloride binding base lead to such an improved selectivity to the compound of formula (I-1) when using the phosphoric acid chloride as starting material.

Thus, the present invention also relates to the use of a combination of a Lewis acid and a hydrogen chloride binding base which is not N-methylimidazole for improving the selectivity to the compound of formula (I-1)

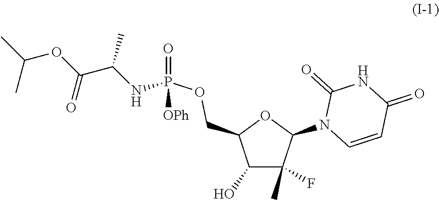 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

of the reaction of a compound of a compound of formula (III)

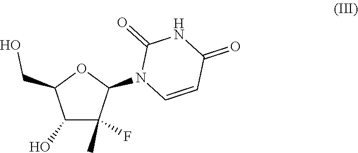 [SMILES: C[C@@]1(F)[C@H](O)[C@@H](CO)O[C@H]1n1ccc(=O)[nH]c1=O]

with a compound of formula (II)

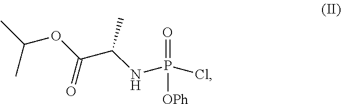 [SMILES: CC(C)OC(=O)[C@H](C)NP(=O)(Cl)Oc1ccccc1]

said compound of formula (II) comprising a compound of formula (I1)

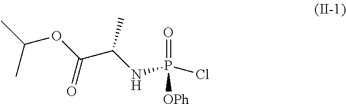 [SMILES: CC(C)OC(=O)[C@H](C)N[P@](=O)(Cl)Oc1ccccc1]

and a compound of formula (II-2)

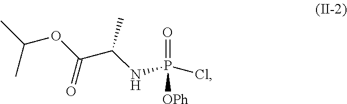 [SMILES: CC(C)OC(=O)[C@H](C)N[P@](=O)(Cl)Oc1ccccc1]

wherein the molar ratio of the compound of formula (II-1) relative the compound of formula (II-2) is in the range of from 55:45 to 45:55, preferably in the range of from 52:48 to 48:52, more preferably in the range of from 51:49 to 49:51, wherein more preferably, the molar ratio of the compound of formula (II-1) relative the compound of formula (II-2) is 1:1. Regarding further preferred combinations, reference is made to the disclosure above, in particular regarding preferred molar ratios disclosed above and preferred hydrogen chloride bases and preferred Lewis bases. Further reference is made to the respective disclosure in the embodiment section of the present application. According to preferred respective use of the present invention, the hydrogen chloride binding base is triethylamine and the Lewis acid is ZnBr₂. Further, the present invention relates to a method for improving the selectivity to the compound of formula (I-1)

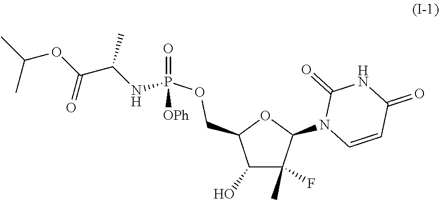 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

of the reaction of a compound of a compound of formula (III)

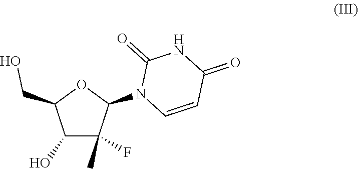 [SMILES: C[C@@]1(F)[C@H](O)[C@@H](CO)O[C@H]1n1ccc(=O)[nH]c1=O]

with a compound of formula (II)

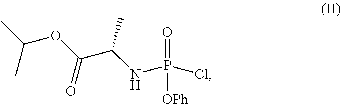 [SMILES: CC(C)OC(=O)[C@H](C)NP(=O)(Cl)Oc1ccccc1]

said compound of formula (II) comprising a compound of formula (II-1)

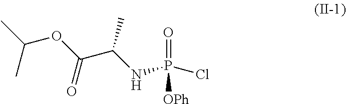 [SMILES: CC(C)OC(=O)[C@H](C)N[P@](=O)(Cl)Oc1ccccc1]

and a compound of formula (II-2)

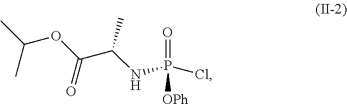 [SMILES: CC(C)OC(=O)[C@H](C)N[P@](=O)(Cl)Oc1ccccc1]

wherein the molar ratio of the compound of formula (II-1) relative the compound of formula (II-2) is in the range of from 55:45 to 45:55, preferably in the range of from 52:48 to 48:52, more preferably in the range of from 51:49 to 49:51, wherein more preferably, the molar ratio of the compound of formula (II-1) relative the compound of formula (II-2) is 1:1, wherein said method comprises employing a combination of a Lewis acid and a hydrogen chloride binding base which is not N-methylimidazole as starting material in said reaction.

Further preferred mixtures, compounds, and uses are explicitly mentioned in the embodiment section hereinbelow.

The present invention related to process carried out in the presence of a base and a Lewis acid is further illustrated by the following embodiments and combinations of embodiments as given by the respective dependencies and references.
1. A process for preparing of a compound of formula (I)

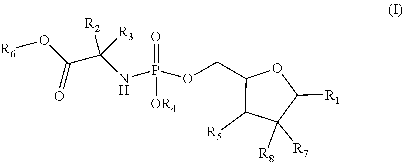

or a salt thereof, the process comprising
a) reacting a compound of formula (II)

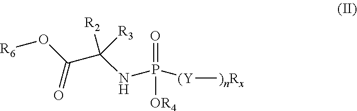

wherein (Y—)ₙRₓ is a leaving group for nucleophilic substitution reaction, wherein n is 0 or 1 and wherein Y is O, N or S, with a compound of formula (III)

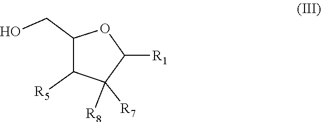

in the presence of a base and a Lewis acid and obtaining a mixture comprising the compound of formula (I).
2. The process of embodiment 1, wherein
   R₄ is phenyl, naphthyl, quinolinyl, isoquinolinyl, quinazolinyl or quinoxalinyl, each optionally substituted with at least one of C₁-C₆ alkyl, C₁-C₆ alkoxy, C₃-C₆ cycloalkyl, aryl, halogen, C(O)OH, CHO, C(O)(C₁-C₆ alkyl), C(O)(aryl), C(O)O(C₁-C₆ alkyl), C(O)ONH₂, C(O)ONH(C₁-C₆ alkyl) and CN;
   R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, NO₂, C(O)OH, CHO, C(O)(C₁-C₆ alkyl), C(O)(aryl), C(O)O(C₁-C₆ alkyl), C(O)ONH₂, C(O)ONH(C₁-C₆ alkyl) and CN;
   R₆ is C₁-C₆ alkyl or C₃-C₁₀ cycloalkyl optionally substituted with at least one of C₁-C₆ alkyl and aryl;
   R₁ is an optionally derivatized purinyl residue, including an adenine residue and a guanine residue, or an optionally derivatized pyrimidinyl residue, including a cytosine residue, a thymine residue and an uracil residue, linked to the furanose ring according to formula (III) through a carbon or nitrogen atom;
   R₇ and R₈ are independently H, OH, F, Cl, Br, I, azide, nitrile, NH₂, NHR₂₆, NR₂₆R₂₄, C(O)NH₂, C(O)NHR₂₆, C(O)NR₂₆R₂₄, C₁-C₆ alkyl optionally substituted with C₁-C₆ alkyl, or C₃-C₁₀ cycloalkyl optionally substituted with C₁-C₆ alkyl, wherein R₂₆ and R₂₄ are independently C₁-C₆ alkyl;
   R₅ is H, OH, C₁-C₆ alkoxy, OC(O)R₂₅, or C₁-C₆ alkyl optionally substituted with C₁-C₆ alkyl or aryl, wherein R₂₅ is C₁-C₆ alkyl or aryl and wherein when n is 1,
   Rₓ is alkyl, aryl, or heteroaryl, each optionally substituted with one or more electron-withdrawing groups, preferably aryl optionally substituted with one or more electron-withdrawing groups, more preferably phenyl optionally substituted with one or more electron-withdrawing groups, more preferably phenyl substituted with one or more electron-withdrawing groups, wherein the one or more electron-withdrawing groups are preferably F, Cl, Br, I, or NO₂; or
   Rₓ is a residue of formula (A)

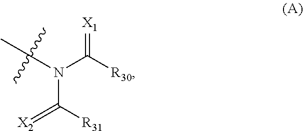

a residue of formula (B)

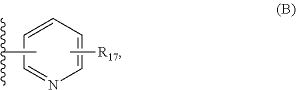

a residue of formula (C)

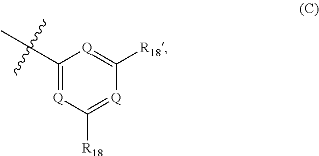

or a residue of formula (D)

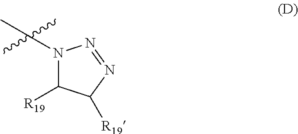

and wherein, when n is 0,
Rₓ is a residue of formula (A1)

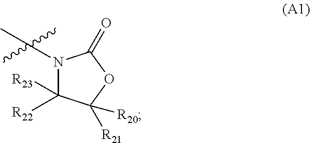

wherein at each occurrence
X₁ and X₂ are independently O or S;
R₃₀ and R₃₁ are independently H, OH, NH₂, C₁-C₆ alkyl or C₁-C₆ alkoxy, or
R₃₀ and R₃₁, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group, preferably F, Cl, Br, I, NO₂, CHO, COOH, COO—(C₁-C₆)alkyl, CN, or COCl;
R₁₈ and R_{18′} are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl;
R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.
3. The process of embodiment 1 or 2, wherein, when n is 1, Rₓ is selected form a residue of formula (A), a residue of formula (B), a residue of formula (C), a residue of formula (D), or when n is 0, Rₓ is selected form a residue of formula (A1).
4. The process of any one of embodiments 1 to 3, wherein n is 0 and Rₓ is a residue of formula (A1)

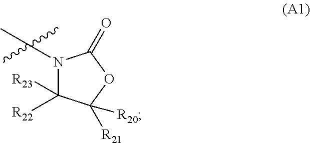

R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.
5. The process of embodiment 2 or 4, wherein the substituent of the optionally substituted 5-, 6-, or 7-membered saturated or partially unsaturated or aromatic ring which is an aryl, preferably benzo, or a heterocycle comprising one or more heteroatoms independently being N, O or S, is at least a substituent, preferably one substituent, selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
6. The process of embodiment 5, wherein the aromatic ring is a benzo substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
7. The process of embodiment 2 or 4, wherein R₂₂ and R₂₃ are each independently H, aryl, or C₁-C₆ alkyl substituted with at least one of C₁-C₆ alkoxy optionally substituted with at least one of OH and NH₂.
8. The process of any one of embodiments 1 to 3, wherein n is 1 and R_{R} is a residue of formula (A)

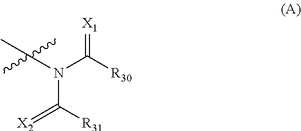

wherein
X₁ and X₂ are independently O or S;
R₃₀ and R₃₁ are independently H, OH, NH₂, C₁-C₆ alkyl or C₁-C₆ alkoxy, or
R₃₀ and R₃₁, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S.
9. The process of any one of embodiments 1 to 3 and 8, wherein Rₓ is a residue of formula (IIb)

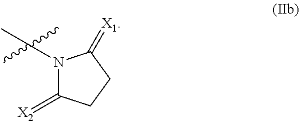

10. The process of any one of embodiments 1 to 3 and 8, wherein Rₓ is a residue of formula (IIc)

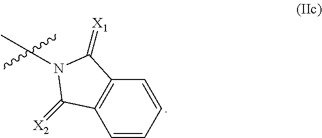

11. The process of any one of embodiments 1 to 3, and 8 to 10, wherein X₁ is O and X₂ is O.
12. The process of any one of embodiments 1 to 3, wherein n is 1 and Rₓ is a residue of formula (B)

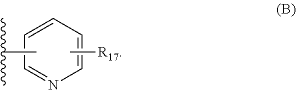

13. The process of any one of embodiments 1 to 3, and 12, wherein R₁₇ is selected from the group consisting of F, Cl, Br, I, NO₂, CHO, COOH, COO—(C₁-C₆)alkyl, CN and COCl.
14. The process of any one of embodiments 1 to 3, wherein n is 1 and Rₓ a residue of formula (C)

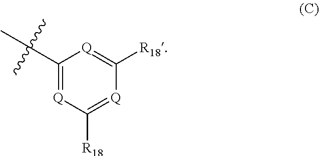

15. The process of any one of embodiments 1 to 3, and 14, wherein R₁₈ and R₁₈, are independently F, Cl, Br, I, or C₁-C₆ alkoxy and each Q is independently C or N, wherein at least one Q is N.
16. The process of any one of embodiments 1 to 3, wherein n is 1 and Rₓ or a residue of formula (D)

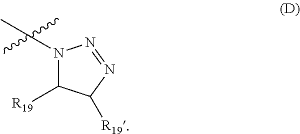

R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the aromatic ring is preferably benzo,
wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl.
17. The process of any one of embodiments 1 to 3, and 16, wherein the substituent of the optionally substituted 5-, 6-, or 7-membered saturated or partially unsaturated or aromatic ring is at least a substituent, preferably one substituent, selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
18. The process of any one of embodiments 1 to 3, and 16 to 17, wherein the aromatic ring formed by R₁₉ and R₁₉, taken together is a benzo substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
19. The process of embodiment 1, wherein n is 0 and Rₓ is Cl and wherein the base is preferably not N-methylimidazole.
20. The process of any one of embodiments 1 to 3, and 8 to 9, for preparing of a compound of formula (I)

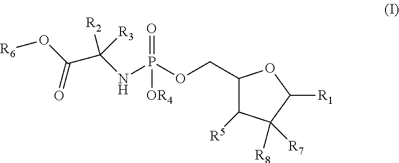

or a salt thereof, the process comprising
a) reacting a compound of formula (II)

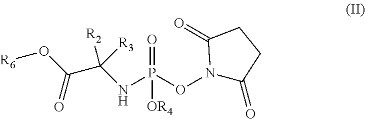

with a compound of formula (III)

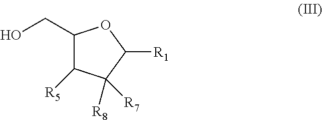

in the presence of a base and a Lewis acid and obtaining a mixture comprising the compound of formula (I).
21. The process of any one of embodiments 1 to 20, wherein the compound of formula (II) comprises a compound of formula (II-A),

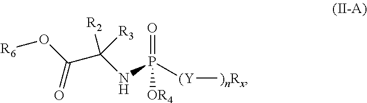

and a compound of formula (II-B),

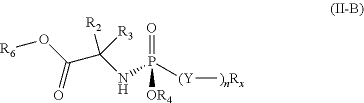

wherein the molar ratio of the compound of formula (II-A) relative the compound of formula (II-B) is in the range of from 45:55 to 72:28, preferably in the range of from 45:55 to 60:40, more preferably in the range of from 45:55 to 55:45.
22. The process of embodiment 21, wherein the molar ratio of the compound of formula (II-A) relative the compound of formula (II-B) is in the range of from 55:45 to 45:55, preferably in the range of from 52:48 to 48:52, more preferably in the range of from 51:49 to 49:51.
23. The process of embodiment 21 or 22, wherein the molar ratio of the compound of formula (II-A) relative the compound of formula (II-B) is 1:1.
24. The process of any one of embodiments 1 to 3, 8 to 9, and 20 to 23, wherein the compound of formula (II) is a compound of formula

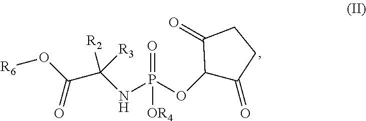

the compound of formula (II-A) is a compound of formula

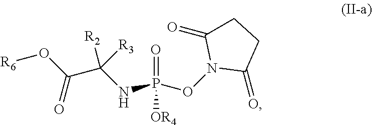

and the compound of formula (II-B) is a compound of formula

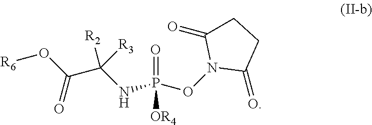

25. The process of any one of embodiments 1 to 3, 8 to 9, and 20 to 24, wherein the compound of formula (II) is a compound of formula (II-0)

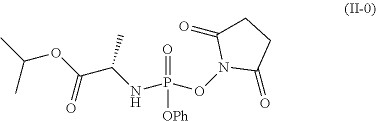 [SMILES: CC(C)OC(=O)[C@H](C)NP(=O)(Oc1ccccc1)ON1C(=O)CCC1=O]

wherein the compound of formula (II-0) comprises, preferably consists of, a compound of formula (II-a)

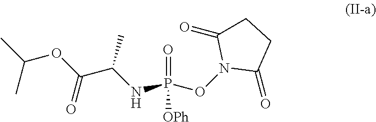 [SMILES: CC(C)OC(=O)[C@H](C)N[P@](=O)(Oc1ccccc1)ON1C(=O)CCC1=O]

and a compound of formula (II-b)

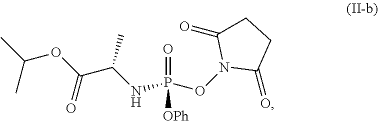 [SMILES: CC(C)OC(=O)[C@H](C)N[P@@](=O)(Oc1ccccc1)ON1C(=O)CCC1=O]

wherein the molar ratio of the compound of formula (II-a) relative the compound of formula (II-b) is in the range of from 45:55 to 72:28, preferably in the range of from 45:55 to 60:40, more preferably in the range of from 45:55 to 55:45.
26. The process of embodiment 25, wherein the molar ratio of the compound of formula (II-a) relative the compound of formula (II-b) is in the range of from 55:45 to 45:55, preferably in the range of from 52:48 to 48:52, more preferably in the range of from 51:49 to 49:51.
27. The process of embodiment 25 or 26, wherein the molar ratio of the compound of formula (II-a) relative the compound of formula (II-b) is 1:1.
28. The process of any one of embodiments 1 to 27, wherein in the mixture obtained from a), the compound of formula (I) comprises, preferably consists of, a compound of formula (I-1)

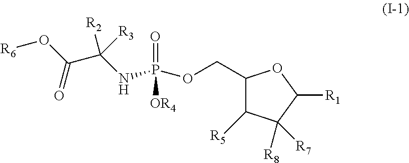

and a compound of formula (I-2)

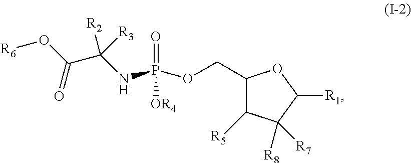

wherein the molar ratio of the compound of formula (I-1) relative to the compound of formula (I-2) is at least 65:35, preferably at least 70:30, more preferably at least 75:25, more preferably at least 80:20, more preferably at least 85:15, more preferably at least 90:10, more preferably at least 95:5, more preferably at least 99:1, more preferably at least 99.8:0.2; or wherein the molar ratio of the compound of formula (I-1) relative to the compound of formula (I-2) is in the range of from 95:5 to 99.8:0.2, preferably in the range of from 97:3 to 99.8:0.2, more preferably in the range of from 99:1 to 99.8:0.2, more preferably is 99.8:0.2.
29. The process of embodiment 28, wherein the molar ratio of the compound of formula (I-1) relative to the compound of formula (I-2) is in the range of from 65:35 to 90:10, preferably in the range of from 75:25 to 90:10, more preferably in the range of from 85:15 to 90:10.
30. The process of embodiment 28 or 29, wherein the compound of formula (II-a) is a compound of formula

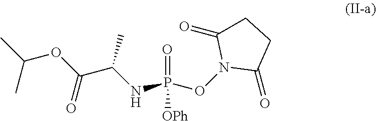 [SMILES: CC(C)OC(=O)[C@H](C)N[P@](=O)(Oc1ccccc1)ON1C(=O)CCC1=O]

and the compound of formula (II-b) is a compound of formula

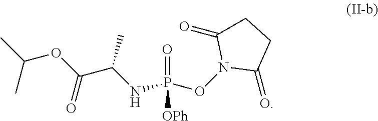 [SMILES: CC(C)OC(=O)[C@H](C)N[P@@](=O)(Oc1ccccc1)ON1C(=O)CCC1=O]

31. The process of any one of embodiments 1 to 30, wherein the compound of formula (III) is a compound of formula

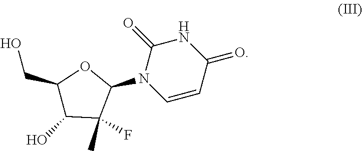 [SMILES: C[C@@]1(F)[C@H](O)[C@@H](CO)O[C@H]1n1ccc(=O)[nH]c1=O]

32. The process of any one of embodiments 1 to 31, wherein R₇ and R₈ are independently H, F, OH or methyl, wherein the compound of formula (III) is preferably

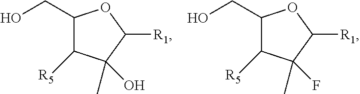

more preferably

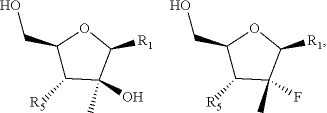

more preferably

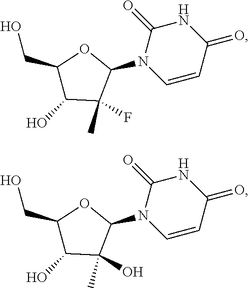

33. The process of embodiment 32, wherein the compound of formula (I-1) is a compound

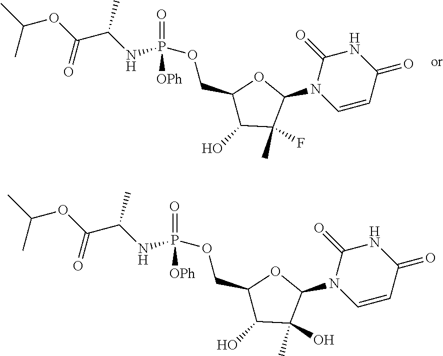 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1 ; CC(C)OC(=O)[C@H](C)N[P@](=O)(OC[C@H]1O[C@@H](n2ccc(=O)[nH]c2=O)[C@@](C)(O)[C@@H]1O)Oc1ccccc1]

and the compound of formula (I-2) is a compound

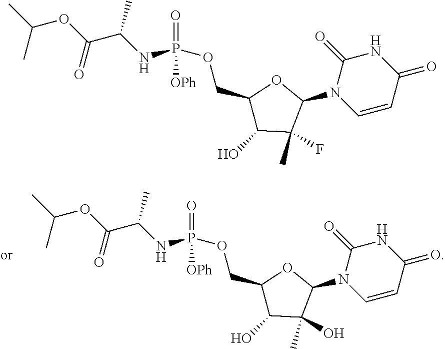 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1 ; CC(C)OC(=O)[C@H](C)N[P@@](=O)(OC[C@H]1O[C@@H](n2ccc(=O)[nH]c2=O)[C@@](C)(O)[C@@H]1O)Oc1ccccc1]

34. A process for preparing of a compound of formula (I)

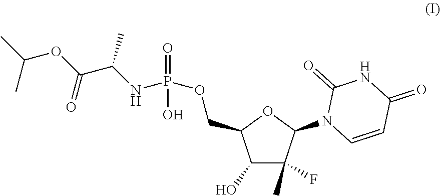 [SMILES: CC(C)OC(=O)[C@H](C)NP(=O)(O)OC[C@H]1O[C@@H](n2ccc(=O)[nH]c2=O)[C@](C)(F)[C@@H]1O]

or a salt thereof, the process comprising
a) reacting a compound of formula (II-0)

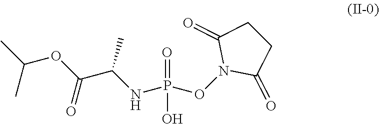 [SMILES: CC(C)OC(=O)[C@H](C)NP(=O)(O)ON1C(=O)CCC1=O]

with a compound of formula (III)

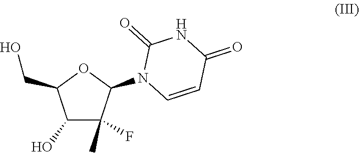 [SMILES: C[C@@]1(F)[C@H](O)[C@@H](CO)O[C@H]1n1ccc(=O)[nH]c1=O]

in the presence of a base and a Lewis acid
obtaining a mixture comprising the compound of formula (I),
wherein the compound of formula (II-0) comprises a compound of formula (II-a)

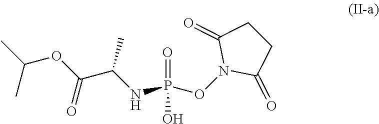 [SMILES: CC(C)OC(=O)[C@H](C)N[P@](=O)(O)ON1C(=O)CCC1=O]

and a compound of formula (II-b)

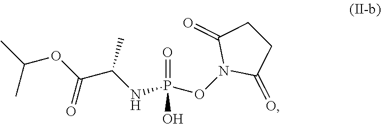 [SMILES: CC(C)OC(=O)[C@H](C)N[P@@](=O)(O)ON1C(=O)CCC1=O]

wherein the molar ratio of the compound of formula (II-a) relative the compound of formula (II-b) is in the range of from 45:55 to 72:28, preferably in the range of from 45:55 to 60:40, more preferably in the range of from 45:55 to 55:45.
35. The process of embodiment 34, wherein the molar ratio of the compound of formula (II-a) relative the compound of formula (II-b) is in the range of from 55:45 to 45:55, preferably in the range of from 52:48 to 48:52, more preferably in the range of from 51:49 to 49:51.
36. The process of embodiment 34 or 35, wherein the molar ratio of the compound of formula (II-a) relative the compound of formula (II-b) is 1:1.
37. The process of any one of embodiments 34 to 36, wherein the compound of formula (II-0) consists of the compound of formula (II-a) and of the compound of formula (II-b).
38. The process of any one of embodiments 34 to 37, wherein in the mixture obtained from a), the compound of formula (I) comprises a compound of formula (I-1)

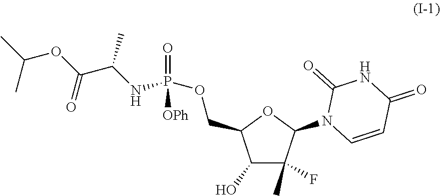 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and a compound of formula (I-2)

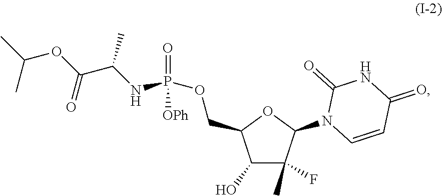 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

wherein the molar ratio of the compound of formula (I-1) relative to the compound of formula (I-2) is at least 65:35, preferably at least 70:30, more preferably at least 75:25, more preferably at least 80:20, more preferably at least 85:15, more preferably at least 90:10, more preferably at least 95:5, more preferably at least 99:1, more preferably at least 99.8:0.2; or wherein the molar ratio of the compound of formula (I-1) relative to the compound of formula (I-2) is in the range of from 95:5 to 99.8:0.2, preferably in the range of from 97:3 to 99.8:0.2, more preferably in the range of from 99:1 to 99.8:0.2, more preferably is 99.8:0.2.
39. The process of embodiment 38, wherein the molar ratio of the compound of formula (I-1) relative to the compound of formula (I-2) is in the range of from 65:35 to 90:10, preferably in the range of from 75:25 to 90:10, more preferably in the range of from 85:15 to 90:10.
40. The process of embodiment 38 or 39, wherein the molar ratio of the compound of formula (I-1) relative to the compound of formula (I-2) is in the range of from 85:15 to 90:10.
41. The process of any one of embodiments 38 to 40, wherein in the mixture obtained from a), the compound of formula (I) consists of a compound of formula (I-1) and a compound of formula (I-2).
42. The process of any one of embodiments 1 to 41, preferably of any one of embodiments 34 to 41, wherein the base is an organic base, preferably an organic nitrogenous base, more preferably a tertiary organic nitrogenous base.
43. The process of embodiment 42, wherein the organic base comprises one or more of an amine, an amidine, and a heteroaromatic compound comprising a basic ring-nitrogen atom.
44. The process of embodiment 42 or 43, wherein the organic base comprises, preferably consists of one or more of ethyldiisopropylamine, triethylamine, diethylamine, 1,8-diazabicycloundec-7-ene, pyridine, quinoline, isoquinoline, acridine, pyrazine, imidazole, benzimidazole, ephedrine, piperidine, tetramethylguanidine and pyrazole, preferably, the organic base consists of one or more of ethyldiisopropylamine, triethylamine, diethylamine, 1,8-diazabicycloundec-7-ene, pyridine, ephedrine, piperidine, tetramethylguanidine.
45. The process of any one of embodiments 1 to 44, preferably of any one of embodiments 34 to 44, wherein the base comprises, preferably consists of one or more of ethyldiisopropylamine, triethylamine, 1,8-diazabicycloundec-7-ene, ephedrine, piperidine, and tetramethylguanidine, preferably ethyldiisopropylamine.
46. The process of any one of embodiments 1 to 45, preferably of any one of embodiments 34 to 45, wherein prior to the reaction according to a), the molar ratio of the base relative to the compound of formula (III) is in the range of from 0.1:1 to 5:1.
47. The process of any one of embodiments 1 to 46, preferably of any one of embodiments 34 to 46, wherein prior to the reaction according to a), the molar ratio of the base relative to the compound of formula (III) is in the range of from 0.5:1 to 5:1, preferably in the range of from 1:1 to 5:1, more preferably in the range of from 2:1 to 5:1, more preferably in the range of from 3:1 to 5:1.
48. The process of any one of embodiments 1 to 47, preferably of any one of embodiments 34 to 47, wherein prior to the reaction according to a), the molar ratio of the base relative to the compound of formula (III) is in the range of from 2:1 to 4:1, preferably in the range of from 2.5:1 to 4:1, more preferably in the range of from 2.5:1 to 3.5:1.
49. The process of any one of embodiments 1 to 48, preferably of any one of embodiments 34 to 48, wherein prior to the reaction according to a), the molar ratio of the base relative to the compound of formula (III) is in the range of from 1:1 to 3:1.
50. The process of any one of embodiments 1 to 49, preferably of any one of embodiments 34 to 49, wherein according to a), the compound of formula (II) or of formula (II-0) is reacted with the compound of formula (III) in the presence of the base and in the presence of the Lewis acid.
51. The process of any one of embodiments 1 to 50, preferably of any one of embodiments 34 to 50, wherein the Lewis acid comprises a twice positively charged ion or a three times positively charged ion.
52. The process of any one of embodiments 1 to 51, preferably of any one of embodiments 34 to 51, wherein the Lewis acid comprises a twice positively charged metal ion or a three times positively charged metal ion.
53. The process of any one of embodiments 1 to 52, preferably of any one of embodiments 34 to 52, wherein the twice positively charged ion is a Zn ion, a Mg ion, a Cu ion, or an Fe ion.
54. The process of any one of embodiments 1 to 53, preferably of any one of embodiments 34 to 53, wherein the twice positively charged ion is a Zn ion.
55. The process of any one of embodiments 1 to 54, preferably of any one of embodiments 34 to 54, wherein the Lewis acid comprises, preferably is, one or more of ZnBr₂, ZnCl₂, and ZnI₂.
56. The process of any one of embodiments 1 to 55, preferably of any one of embodiments 34 to 55, wherein the Lewis acid comprises, preferably is, ZnBr₂.
57. The process of any one of embodiments 1 to 56, preferably of any one of embodiments 34 to 56, wherein the Lewis acids is one or more of ZnBr₂, ZnCl₂, ZnI₂, MgBr₂, MgBr₂.OEt₂, CuCl₂, Cu(acetylacetonate)₂, and Fe(II) fumarate.
58. The process of embodiment 51 or 52, wherein the three times positively charged ion is a Mn ion.
59. The process of embodiment 58, wherein the Lewis acid comprises, preferably is, Mn(acetylacetonate)₃.
60. The process of any one of embodiments 1 to 59, preferably of any one of embodiments 34 to 59, wherein prior to the reaction according to a), the molar ratio of the Lewis acid relative to the compound of formula (III) is in the range of from 0.1:1 to 5:1.
61. The process of any one of embodiments 1 to 60, preferably of any one of embodiments 34 to 60, wherein prior to the reaction according to a), the molar ratio of the Lewis acid relative to the compound of formula (III) is in the range of from 0.2:1 to 5:1, preferably in the range of from 0.5:1 to 3:1, more preferably in the range of from 0.75:1 to 1.5:1.
62. The process of any one of embodiments 1 to 61, preferably of any one of embodiments 34 to 61, wherein prior to the reaction according to a), the molar ratio of the compound of formula (II) relative to the compound of formula (III) is in the range of from 0.5:1 to 5:1, is in the range of 0.5 to 6:1.
63. The process of any one of embodiments 1 to 62, preferably of any one of embodiments 34 to 62, wherein the molar ratio of the compound of formula (II) relative to the compound of formula (III) is in the range of from 0.8:1 to 2:1, preferably in the range of from 0.9:1 to 1.2:1.
64. The process of any one of embodiments 1 to 63, preferably of any one of embodiments 34 to 63, wherein the molar ratio of the compound of formula (II) relative to the compound of formula (III) is in the range of from 1.4:1 to 6:1, preferably in the range of from 1.4:1 to 4.9:1, more preferably in the range of from 2.1:1 to 5.5:1; more preferably in the range of from 2.1:1 to 4.9:1, more preferably in the range of from 3:1 to 5:1, more preferably in the range of from 3:1 to 4.9:1, more preferably in the range of from 3:1 to 4:1.
65. The process of any one of embodiments 1 to 64, preferably of any one of embodiments 34 to 64, wherein according to a), the compound of formula (II) is reacted with the compound of formula (III) in the presence of the base and a Lewis acid, and in the presence of a solvent.
66. The process of embodiment 65, wherein the solvent comprises, preferably is, one or more organic solvents.
67. The process of embodiment 65 or 66, wherein the solvent comprises, preferably is, one or more aprotic organic solvents.
68. The process of any one of embodiments 65 to 67, wherein the solvent comprises one or more of dichloromethane, methyl tert-butyl ether, tetrahydrofuran, dimethylsulphoxide, and dimethylformamide.
69. The process of any one of embodiments 65 to 68, wherein the solvent comprises, preferably is, tetrahydrofuran.
70. The process of any one of embodiments 1 to 69, preferably of any one of embodiments 34 to 69, wherein the reacting according to a) is carried out at a temperature in the range of from 0 to 80° C., preferably in the range of from 0 to 25° C.
71. The process of any one of embodiments 1 to 70, preferably of any one of embodiments 34 to 70, wherein the reacting according to a) is carried out at a temperature in the range of from 16 to 25° C.
72. The process of any one of embodiments 1 to 71, preferably of any one of embodiments 34 to 71, wherein the reacting according to a) is carried out at a temperature in the range of from 0 to 10° C. or at a temperature in the range of from 0 to 8° C. or at a temperature in the range of from 0 to 5° C.
73. The process of any one of embodiments 1 to 72, preferably of any one of embodiments 34 to 72, wherein the reacting according to a) is carried out at a temperature in the range of from 0 to 8° C. or at a temperature in the range of from 0 to 5° C.
74. The process of any one of embodiments 1 to 73, preferably of any one of embodiments 34 to 73, wherein the reacting according to a) is carried out at a temperature in the range of from 0 to 5° C.
75. The process of any one of embodiments 1 to 74, preferably of any one of embodiments 34 to 74, wherein the reacting according to a) is carried out for a period of time in the range of from 0.5 to 48 h, preferably in the range of from 0.75 to 42 h, more preferably in the range of from 1 to 36 h.
76. The process of any one of embodiments 1 to 75, preferably of any one of embodiments 34 to 75, wherein the reacting according to a) is carried out for a period of time in the range of from 2 to 24 h, preferably in the range of from 15 to 24 h.
77. The process of any one of embodiments 1 to 76, preferably of any one of embodiments 34 to 76, further comprising
   b) separating the compound of formula (I) from the mixture obtained in a).
78. The process of embodiment 77, wherein the separating according to b) comprises filtration, centrifugation, drying, or a combination of two or more thereof.
79. The process of embodiment 78, wherein drying comprises drying in an atmosphere comprising oxygen, nitrogen, or a mixture thereof.
80. The process of embodiment 78 or 79, wherein drying comprises rapid-drying, preferably spray-drying.
81. The process of any one of embodiments 77 to 80, wherein the separating in b) comprises separating the compound of formula (I) from the mixture obtained in a), obtaining the compound of formula (I) dissolved in a solvent, preferably an organic solvent.
82. The process of embodiment 81, further comprising
   c) crystallizing the compound of formula (I), preferably from the mixture obtained from b) comprising the compound of formula (I) dissolved in a solvent, wherein the compound (I) comprises, preferably consists of, a compound of formula (I-1) and a compound of formula (I-2).
83. The process of embodiment 82, wherein the crystallizing in c) comprises seeding, preferably seeding with seed crystals of the compound of formula (I-1).
84. The process of embodiment 82 or 83, wherein the separating according to b) comprises
   c) crystallizing the compound of formula (I), preferably from the mixture obtained from b) comprising the compound of formula (I) dissolved in a solvent, obtaining the crystallized compound of formula (I) in its mother liquor;
   d) separating the crystallized compound of formula (I) from its mother liquor, obtaining the compound of formula (I) in crystalline form, said separating preferably comprising
      d1) subjecting the mother liquor comprising the crystallized compound of formula (I) to filtration, obtaining a filter cake comprising the compound of formula (I);
      d2) optionally washing the filter cake comprising the compound of formula (I);
      d3) drying the optionally washed filter cake, obtaining the compound of formula (I).
   e) optionally recrystallizing the compound of formula (I) from d) from a solvent,
   f) optionally separating the crystallized compound of formula (I) from e) from the solvent,
   wherein the compound (I) comprises, preferably consists of, a compound of formula (I-1) and a compound of formula (I-2).
85. The process of any one of embodiments 82 to 84 wherein in c) and in e) the solvent is an organic solvent, more preferably an organic solvent selected from the group consisting of a ketone, an ester, an ether, a C₁-C₇ alkane, a halo-alkane, a nitrile, an aromatic hydrocarbon solvent, an alcohol and a mixture thereof.
86. The process of embodiment 85, wherein the ketone is selected from the group consisting of acetone, methyl isobutyl ketone, diethyl ketone, methyl propyl ketone, methyl isopropyl ketone, acetophenone, diethyl butyl ketone and mixture thereof and wherein the ketone is optionally in combination with a solvent selected from the group consisting of methyl tert-butyl ether, isopropyl acetate and toluene.
87. The process of embodiment 85 or 86, wherein the ether is selected from the group consisting of methyl tert-butyl ether and tetrahydrofuran.
88. The process of any one of embodiments 85 to 87, wherein the ester is selected from the group consisting of ethyl acetate, isopropyl acetate and butyl acetate.
89. The process of any one of embodiments 85 to 88, wherein the C₁-C₇ alkane is selected from a C₅-C₇ alkane wherein the C₅-C₇ alkane is selected from the group consisting of cyclohexane and n-heptane and wherein the C₁-C₇ alkane is optionally in combination with a solvent selected form the group consisting of methyl tert-butyl ether, isopropyl acetate and toluene.
90. The process of any one of embodiment 85 to 89, wherein the halo-alkane is dichloromethane and wherein the halo-alkane is optionally in combination with a solvent selected from the group consisting of toluene, tetrahydrofuran, acetone and methyl isobutyl ketone.
91. The process of any one of embodiments 85 to 90, wherein the nitrile is acetonitrile and wherein the nitrile is optionally in combination with a solvent selected form the group consisting of diisopropyl ether or tert-butyl methyl ether.
92. The process of any one of embodiments 85 to 91, wherein the aromatic hydrocarbon solvent is selected from the group consisting of anisole and toluene.
93. The process of any one of embodiment 85 to 92, wherein the alcohol is selected from a C₁-C₈ alcohol, preferably the alcohol is n-butanol, optionally in combination with heptane, preferably n-heptane.
94. The process of any one of embodiments 85 to 93, wherein the solvent is selected from the group consisting of acetone, methyl isobutyl ketone, diethyl ketone, methyl propyl ketone, methyl isopropyl ketone, acetophenone, diethyl butyl ketone, methyl tert-butyl ether, tetrahydrofuran, ethyl acetate, isopropyl acetate, butyl acetate, cyclohexane, heptane, preferably n-heptane, dichloromethane, acetonitrile, anisole, toluene, n-butanol and mixture thereof, wherein preferably, the solvent is dichloromethane.
95. The process of any one of embodiments 85 to 94, wherein the organic solvent is selected from the group consisting of dichloromethane, acetonitrile and anisole or mixture thereof, wherein preferably, the solvent is dichloromethane.
96. The process of embodiment 95, the molar ratio of the compound of formula (I-1) relative to the compound of formula (I-2) after c) or from d) or from d3) or from e) is increased with respect to the molar ratio of the compound of formula (I-1) relative to the compound of formula (I-2) of a).
97. The process of embodiment 95 or 96, wherein the molar ratio of the compound of formula (I-1) relative to the compound of formula (I-2) after c) or from d) or from d3) or from e) is at least 95:5, preferably at least 97:3, more preferably at least 99:1, more preferably at least 99.8:0.2.
98. The process of any one of embodiment 95 to 97, wherein the molar ratio of the compound of formula (I-1) relative to the compound of formula (I-2), after c) or from d) or from d3) or of e) is in the range of from 95:5 to 99.8:0.2, preferably in the range of from 97:3 to 99.8:0.2, more preferably in the range of from 99:1 to 99.8:0.2.
99. The process of any one of embodiments 95 to 98, wherein the crystallization step c) is carried out at a temperature in the range of from −10 to 50° C.
100. The process of any one of embodiments 95 to 99, wherein the recrystallization step e) is carried out at a temperature in the range of from −10 to 50° C.
101. A mixture comprising a compound of formula (I), obtainable or obtained by a process according to any one of embodiments 1 to 100, preferably of any one of embodiments 34 to 100.
102. A mixture, preferably a mixture of embodiment 101, comprising the compound of formula (I)

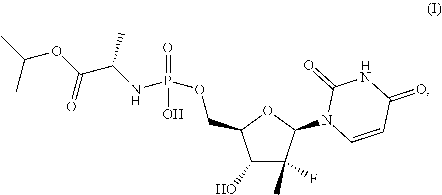 [SMILES: CC(C)OC(=O)[C@H](C)NP(=O)(O)OC[C@H]1O[C@@H](n2ccc(=O)[nH]c2=O)[C@](C)(F)[C@@H]1O]

wherein the compound of formula (I) comprises a compound of formula (I-1)

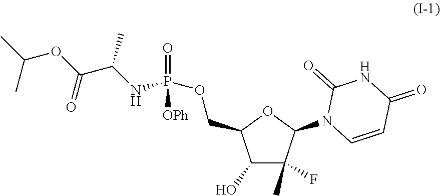 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and a compound of formula (I-2)

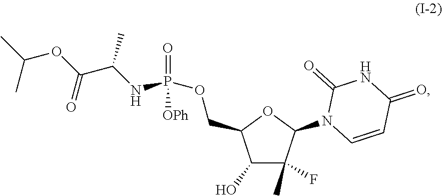 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

wherein the molar ratio of the compound of formula (I-1) relative to the compound of formula (I-2) is at least 65:35, preferably at least 70:30, more preferably at least 75:25, more preferably at least 80:20, more preferably at least 85:15, more preferably at least 90:10, more preferably at least 95:5, more preferably at least 99:1, more preferably at least 99.8:0.2.
103. The mixture of embodiment 101 or 102, wherein the molar ratio of the compound of formula (I-1) relative to the compound of formula (I-2) is in the range of from 65:35 to 90:10, preferably in the range of from 75:25 to 90:10, more preferably in the range of from 85:15 to 90:10.
104. The mixture of embodiment 101 or 102, wherein the molar ratio of the compound of formula (I-1) relative to the compound of formula (I-2) is at least 95:5, preferably at least 97:3, more preferably at least 99:1, more preferably at least 99.8:0.2.
105. The mixture of any one of embodiments 101, 102, and 104 wherein the molar ratio of the compound of formula (I-1) relative to the compound of formula (I-2) is in the range of from 95:5 to 99.8:0.2, preferably in the range of from 97:3 to 99.8:0.2, more preferably in the range of from 99:1 to 99.8:0.2.
106. The mixture of any one of embodiments 101 to 105, wherein the base is an organic base, preferably is an organic nitrogenous base, more preferably is a tertiary organic nitrogenous base.
107. The mixture of embodiment 106, wherein the organic base comprises one or more of an amine, an amidine, and a heteroaromatic compound comprising a basic ring-nitrogen atom.
108. The mixture of embodiment 106 or 107, wherein the organic base comprises one or more of ethyldiisopropylamine, triethylamine, diethylamine, 1,8-diazabicycloundec-7-ene, pyridine, quinoline, isoquinoline, acridine, pyrazine, imidazole, benzimidazole, ephedrine, piperidine, tetramethylguanidine and pyrazole, preferably, the organic base consists of one or more of ethyldiisopropylamine, triethylamine, diethylamine, 1,8-diazabicycloundec-7-ene, pyridine, ephedrine, piperidine, tetramethylguanidine.
109. The mixture of any one of embodiments 101 to 108, wherein the base comprises ethyldiisopropylamine, triethylamine, 1,8-diazabicycloundec-7-ene, ephedrine, piperidine, and tetramethylguanidine, preferably ethyldiisopropylamine.
110. The mixture of any one of embodiments 101 to 109, comprising a Lewis acid preferably comprising a twice positively charged ion or a three times positively charged ion, more preferably a twice positively charged metal ion or a three times positively charged metal ion, more preferably a twice positively charged metal ion preferably being a Zn ion, a Mg ion, a Cu ion, or an Fe ion, preferably a Zn ion, the Lewis acid more preferably comprising, more preferably being, one or more of ZnBr₂, ZnCl₂, and ZnI₂, more preferably comprising, more preferably being, ZnBr₂.
111. The mixture of any one of embodiments 101 to 110, further comprising a solvent preferably comprising one or more organic solvents, more preferably one or more aprotic organic solvents, more preferably comprising one or more of dichloromethane, methyl tert-butyl ether, tetrahydrofuran, dimethylsulphoxide, and dimethylformamide, wherein more preferably, the solvent comprises, more preferably is, tetrahydrofuran.
112. Use of a mixture of any one of embodiments 101 to 111 for obtaining the compound of formula (I)

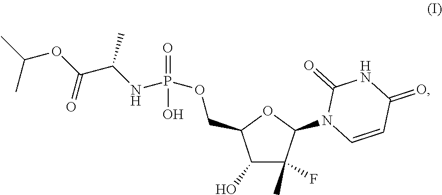 [SMILES: CC(C)OC(=O)[C@H](C)NP(=O)(O)OC[C@H]1O[C@@H](n2ccc(=O)[nH]c2=O)[C@](C)(F)[C@@H]1O]

comprising preferably consisting of a compound of formula (I-1) and a compound of formula (I-2), wherein the molar ratio of the compound of formula (I-1) relative to the compound of formula (I-2) is at least 65:35, preferably at least 70:30, more preferably at least 75:25, more preferably at least 80:20, more preferably at least 85:15, more preferably at least 90:10, more preferably at least 95:5, more preferably at least 99:1, more preferably at least 99.8:0.2.
113. The use of embodiment 112, wherein the molar ratio of the compound of formula (I-1) relative to the compound of formula (I-2) is in the range of from 65:35 to 90:10, preferably in the range of from 75:25 to 90:10, more preferably in the range of from 85:15 to 90:10.
114. The use of embodiment 112 or 113, wherein the molar ratio of the compound of formula (I-1) relative to the compound of formula (I-2) is at least 95:5, preferably at least 97:3, more preferably at least 99:1, more preferably at least 99.8:0.2.
115. The use of any one of embodiments 112 to 114, wherein the molar ratio of the compound of formula (I-1) relative to the compound of formula (I-2) is in the range of from 95:5 to 99.8:0.2, preferably in the range of from 97:3 to 99.8:0.2, more preferably in the range of from 99:1 to 99.8:0.2.
116. The use of embodiment 112, wherein obtaining the compound of formula (I) comprises separating the compound of formula (I) from the mixture and crystallizing the compound of formula (I), obtaining the crystallized compound of formula (I) in its mother liquor and separating the crystallized compound of formula (I) from its mother liquor.
117. A mixture comprising a compound of formula (II-0)

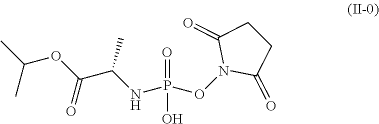 [SMILES: CC(C)OC(=O)[C@H](C)NP(=O)(O)ON1C(=O)CCC1=O]

and a compound of formula (III)

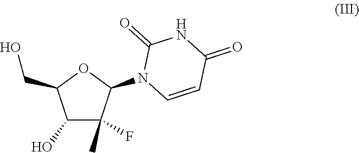 [SMILES: C[C@@]1(F)[C@H](O)[C@@H](CO)O[C@H]1n1ccc(=O)[nH]c1=O]

a base, and Lewis acid, wherein the compound of formula (II-0) comprises a compound of formula (II-a)

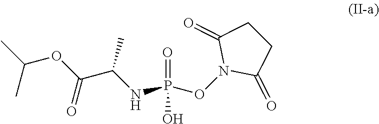 [SMILES: CC(C)OC(=O)[C@H](C)N[P@](=O)(O)ON1C(=O)CCC1=O]

and a compound of formula (II-b)

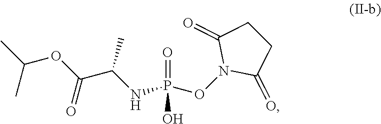 [SMILES: CC(C)OC(=O)[C@H](C)N[P@@](=O)(O)ON1C(=O)CCC1=O]

wherein the molar ratio of the compound of formula (II-a) relative the compound of formula (II-b) is in the range of from 45:55 to 72:28, preferably in the range of from 45:55 to 60:40, more preferably in the range of from 45:55 to 55:45.
118. The mixture of embodiment 117, wherein the molar ratio of the compound of formula (II-a) relative the compound of formula (II-b) is in the range of from 55:45 to 45:55, preferably in the range of from 52:48 to 48:52, more preferably in the range of from 51:49 to 49:51.
119. The mixture of embodiment 117 or 118, wherein the molar ratio of the compound of formula (II-a) relative the compound of formula (II-b) is 1:1.
120. The mixture of any one of embodiments 117 to 119, wherein the base is an organic base, preferably an organic nitrogenous base, more preferably a tertiary organic nitrogenous base.
121. The mixture of embodiment 120, wherein the organic base comprises one or more of an amine, an amidine, and a heteroaromatic compound comprising a basic ring-nitrogen atom.
122. The mixture of embodiment 120 or 121, wherein the organic base comprises, preferably consists of one or more of ethyldiisopropylamine, triethylamine, diethylamine, 1,8-diazabicycloundec-7-ene, pyridine, quinoline, isoquinoline, acridine, pyrazine, imidazole, benzimidazole, ephedrine, piperidine, tetramethylguanidine and pyrazole, preferably, the organic base consists of one or more of ethyldiisopropylamine, triethylamine, diethylamine, 1,8-diazabicycloundec-7-ene, pyridine, ephedrine, piperidine, tetramethylguanidine.
123. The mixture of any one of embodiments 117 to 122, wherein the base comprises, preferably consists of one or more of ethyldiisopropylamine, triethylamine, 1,8-diazabicycloundec-7-ene, ephedrine, piperidine, and tetramethylguanidine, preferably ethyldiisopropylamine.
124. The mixture of any one of embodiments 117 to 123, wherein the molar ratio of the base relative to the compound of formula (III) is in the range of from 0.1:1 to 5:1.
125. The mixture of any one of embodiments 117 to 124, wherein the molar ratio of the base relative to the compound of formula (III) is in the range of from 0.5:1 to 5:1, preferably in the range of from 1:1 to 5:1, more preferably in the range of from 2:1 to 5:1.
126. The mixture of any one of embodiments 117 to 125, wherein the molar ratio of the base relative to the compound of formula (III) is in the range of from 2:1 to 4:1, preferably in the range of from 2.5:1 to 4:1, more preferably in the range of from 2.5:1 to 3.5:1.
127. The mixture of any one of embodiments 117 to 126, wherein the molar ratio of the base relative to the compound of formula (III) is in the range of from 1:1 to 3:1.
128. The mixture of any one of embodiments 117 to 127, comprising a Lewis acid preferably comprising a twice positively charged ion or a three times positively charged ion, more preferably a twice positively charged metal ion or a three times positively charged metal ion, more preferably a twice positively charged metal ion preferably being a Zn ion, a Mg ion, a Cu ion, or an Fe ion, preferably a Zn ion, the Lewis acid more preferably comprising, more preferably being, one or more of ZnBr₂, ZnCl₂, and ZnI₂, more preferably comprising, more preferably being, ZnBr₂.
129. The mixture of any one of embodiments 117 to 128, wherein the molar ratio of the Lewis acid relative to the compound of formula (III) is in the range of from 0.1:1 to 5:1.
130. The mixture of any one of embodiments 1117 to 129, wherein the molar ratio of the Lewis acid relative to the compound of formula (III) is in the range of from 0.2:1 to 5:1, preferably in the range of from 0.5:1 to 3:1, more preferably in the range of from 0.75:1 to 1.5:1.
131. The mixture of any one of embodiments 117 to 130, further comprising a solvent preferably comprising one or more organic solvents, more preferably one or more aprotic organic solvents, more preferably comprising one or more of dichloromethane, methyl tert-butyl ether, tetrahydrofuran, dimethylsulphoxide, and dimethylformamide, wherein more preferably, the solvent comprises, more preferably is, tetrahydrofuran.
132. Use of a mixture according to any one of embodiments 117 to 131 for preparing a compound of formula (I-1)

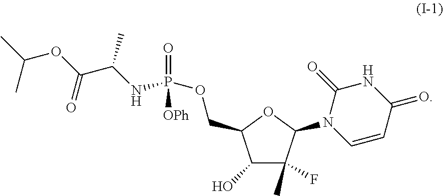 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

133. Use of a mixture according to any one of embodiments 117 to 132 for improving the selectivity to the compound of formula (I-1)

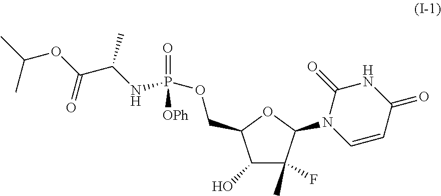 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

of the reaction of compound of formula (III) with a compound of formula (II-0) to the compound of formula (I-1)

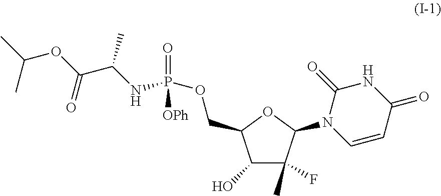 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

wherein the compound of formula (II-0) comprises a compound of formula (II-a) and a compound of formula (II-b) wherein the molar ratio of the compound of formula (II-a) relative the compound of formula (II-b) in the range of from 45:55 to 72:28, preferably in the range of from 45:55 to 60:40, more preferably in the range of from 45:55 to 55:45.
134. The use of embodiment 133, wherein the molar ratio of the compound of formula (II-a) relative the compound of formula (II-b) is in the range of from 55:45 to 45:55, preferably in the range of from 52:48 to 48:52, more preferably in the range of from 51:49 to 49:51.
135. The use of embodiment 133 or 134, wherein the molar ratio of the compound of formula (II-a) relative the compound of formula (II-b) is 1:1.
136. Use of a combination of a Lewis acid and a base for improving the selectivity of the reaction of a compound of formula (III)

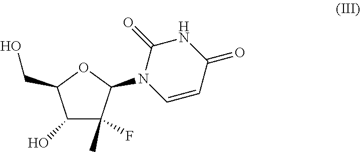 [SMILES: C[C@@]1(F)[C@H](O)[C@@H](CO)O[C@H]1n1ccc(=O)[nH]c1=O]

with a compound of formula (I-0)

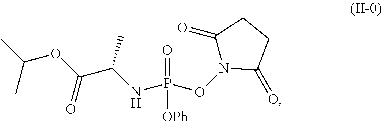 [SMILES: CC(C)OC(=O)[C@H](C)NP(=O)(Oc1ccccc1)ON1C(=O)CCC1=O]

to the compound of formula (I-1)

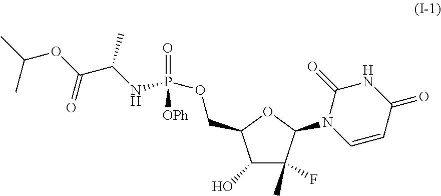 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

obtained from said reaction,
said compound of formula (II-0) comprising a compound of formula (II-0)

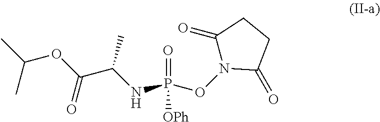 [SMILES: CC(C)OC(=O)[C@H](C)N[P@](=O)(Oc1ccccc1)ON1C(=O)CCC1=O]

and a compound of formula (II-b)

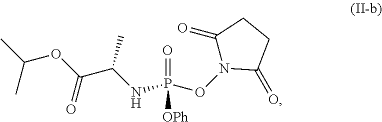 [SMILES: CC(C)OC(=O)[C@H](C)N[P@@](=O)(Oc1ccccc1)ON1C(=O)CCC1=O]

wherein the molar ratio of the compound of formula (II-a) relative the compound of formula (II-b) is in the range of from 45:55 to 72:28, preferably in the range of from 45:55 to 60:40, more preferably in the range of from 45:55 to 55:45.
137. The use of embodiment 136, wherein the molar ratio of the compound of formula (II-a) relative the compound of formula (II-b) is in the range of from 55:45 to 45:55, preferably in the range of from 52:48 to 48:52, more preferably in the range of from 51:49 to 49:51.
138. The use of embodiment 136 or 137, wherein the base is an organic base, preferably an organic nitrogenous base, more preferably a tertiary organic nitrogenous base.
139. The use of embodiment 138, wherein the organic base comprises one or more of an amine, an amidine, and a heteroaromatic compound comprising a basic ring-nitrogen atom.
140. The use of embodiment 138 or 139, wherein the organic base comprises, preferably consists of, one or more of ethyldiisopropylamine, triethylamine, diethylamine, 1,8-diazabicycloundec-7-ene, pyridine, quinoline, isoquinoline, acridine, pyrazine, imidazole, benzimidazole, ephedrine, piperidine, tetramethylguanidine and pyrazole, preferably, the organic base consists of one or more of ethyldiisopropylamine, triethylamine, diethylamine, 1,8-diazabicycloundec-7-ene, pyridine, ephedrine, piperidine, tetramethylguanidine.
141. The use of any one of embodiments 136 to 140, wherein the base comprises, preferably consists of, one or more of ethyldiisopropylamine, triethylamine, 1,8-diazabicycloundec-7-ene, ephedrine, piperidine, and tetramethylguanidine, preferably ethyldiisopropylamine.
142. The use of any one of embodiments 136 to 141, wherein the Lewis acid comprises a twice positively charged ion or a three times positively charged ion, more preferably a twice positively charged metal ion or a three times positively charged metal ion, more preferably a twice positively charged metal ion preferably being a Zn ion, a Mg ion, a Cu ion, or an Fe ion, preferably a Zn ion, the Lewis acid more preferably comprising, more preferably being, one or more of ZnBr₂, ZnCl₂, and ZnI₂, more preferably comprising, more preferably being, ZnBr₂.
143. A compound of formula (I)

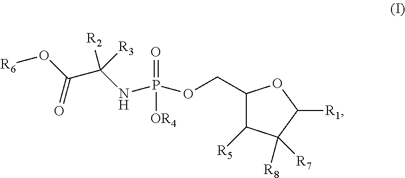

preferably a compound of formula (I)

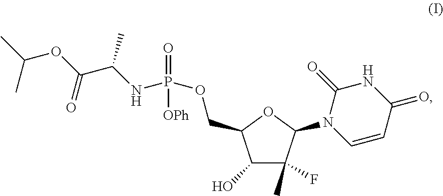 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

more preferably a compound of formula (I-1)

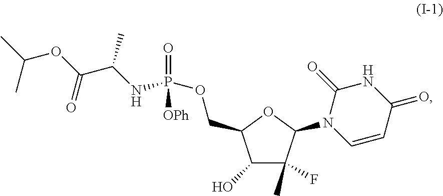 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

obtainable or obtained by a process according to any one of embodiments 1 to 100, preferably according to any one of embodiments 77 to 100, more preferably according to any one of embodiments 84 to 98.
144. Use of the compound of embodiment 143 for the preparation of a pharmaceutical composition.
145. A method of using the compound of embodiment 143 for the preparation of a pharmaceutical composition.
146 A pharmaceutical composition, comprising the compound of embodiment 143 and preferably at least one pharmaceutically acceptable excipient.
147. The pharmaceutical composition of embodiment 146 for use in a method for treating hepatitis C in a human.
148. Use of the pharmaceutical composition of embodiment 146 or 147 for treating hepatitis C in a human.
149. A method of treating hepatitis C in a human, comprising administering the pharmaceutical composition of embodiments 146 or 147 to a human.
150. Use of the crystalline form of sofosbuvir obtainable or obtained by a process according to any of one embodiments 84 to 100, for preparing a medicament for the treatment hepatitis C in a human.
151. Use of the compound of embodiment 143, for the treatment of hepatitis C in a human.
150. The compound of embodiment 143 for use in the treatment of hepatitis C in a human.
151. The compound of embodiment 143 for the treatment of hepatitis C in a human.
152. A method of treating hepatitis C in a human comprising administering the compound of embodiment 143 to a human.
153. The process of any one of embodiments 1 to 100, wherein n is 0 and Rₓ is Cl and wherein the base preferably is not tert-butylmagnesium chloride, more preferably is not an in-organic and organic alkali selected from sodium hydride, tert-butylmagnesium chloride, lithium hydride, lithium tert-butoxide, potassium tert-butoxide, sodium tert-butoxide, NaHMDS, LiHMDS, methylimidazole, 1-methylimidazole, 2-methylimidazole, 4-methylimidazoles, and DBU.

The present invention, relating to a process wherein Cl is used as a leaving group, is further illustrated by the following embodiments and combinations of embodiments resulting from the given dependencies and back-references:
1′. A process for preparing of a compound of formula (I)

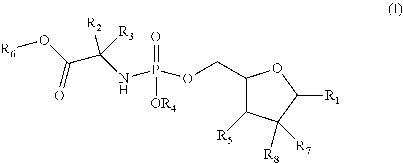

or a salt thereof, the process comprising
a′) reacting a compound of formula (II)

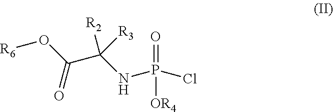

with a compound of formula (III)

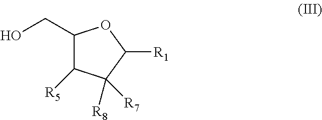

in the presence of a hydrogen chloride binding base, obtaining a mixture comprising the compound of formula (I), wherein preferably, the hydrogen chloride binding base is not, more preferably does not comprise, N-methylimidazole.
2′. The process of embodiment 1′, wherein
   R₄ is phenyl, naphthyl, quinolinyl, isoquinolinyl, quinazolinyl or quinoxalinyl, each optionally substituted with at least one of C₁-C₆ alkyl, C₁-C₆ alkoxy, C₃-C₆ cycloalkyl, aryl, halogen, C(O)OH, CHO, C(O)(C₁-C₆ alkyl), C(O)(aryl), C(O)O(C₁-C₆ alkyl), C(O)ONH₂, C(O)ONH(C₁-C₆ alkyl) and CN;
   R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, NO₂, C(O)OH, CHO, C(O)(C₁-C₆ alkyl), C(O)(aryl), C(O)O(C₁-C₆ alkyl), C(O)ONH₂, C(O)ONH(C₁-C₆ alkyl) and CN;
   R₆ is C₁-C₆ alkyl or C₃-C₁₀ cycloalkyl optionally substituted with at least one of C₁-C₆ alkyl and aryl;
   R₁ is an optionally derivatized purinyl residue, including an adenine residue and a guanine residue, or an optionally derivatized pyrimidinyl residue, including a cytosine residue, a thymine residue and an uracil residue, linked to the furanose ring according to formula (III) through a carbon or nitrogen atom;
   R₇ and R₈ are independently H, OH, F, Cl, Br, I, azide, nitrile, NH₂, NHR₂₆, NR₂₆R₂₄, C(O)NH₂, C(O)NHR₂₆, C(O)NR₂₆R₂₄, C₁-C₆ alkyl optionally substituted with C₁-C₆ alkyl, or C₃-C₁₀ cycloalkyl optionally substituted with C₁-C₆ alkyl, wherein R₂₆ and R₂₄ are independently C₁-C₆ alkyl;
   R₉ is H, OH, C₁-C₆ alkoxy, OC(O)R₂₅, or C₁-C₆ alkyl optionally substituted with C₁-C₆ alkyl or aryl, wherein R₂₅ is C₁-C₆ alkyl or aryl.
3′. The process of embodiment 1′ or 2′, wherein the compound of formula (II) comprises, preferably consists of, a compound of formula (II-1)

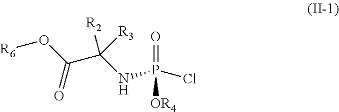

and a compound of formula (II-2)

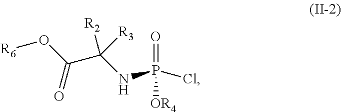

wherein the molar ratio of the compound of formula (II-1) relative the compound of formula (II-2) is preferably in the range of from 55:45 to 45:55, more preferably in the range of from 52:48 to 48:52, more preferably in the range of from 51:49 to 49:51.
4′. The process of embodiment 3′, wherein the molar ratio of the compound of formula (II-1) relative the compound of formula (II-2) is 1:1.
5′. The process of embodiment 3′ or 4′, wherein in the mixture obtained from a′), the compound of formula (I) comprises, preferably consists of, a compound of formula (I-1)

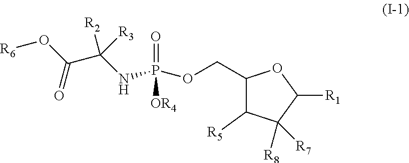

and a compound of formula (I-2)

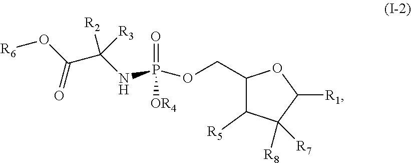

wherein the molar ratio of the compound of formula (I-1) relative to the compound of formula (I-2) is at least 65:35, preferably at least 70:30, more preferably at least 75:25.
6′. The process of embodiment 5′, wherein the molar ratio of the compound of formula (I-1) relative to the compound of formula (I-2) is at least 80:20.
7′. The process of embodiment 5′ or 6′, wherein the compound of formula (II-1) is a compound of formula

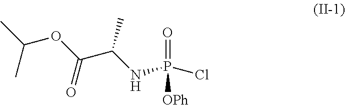 [SMILES: CC(C)OC(=O)[C@H](C)N[P@](=O)(Cl)Oc1ccccc1]

and the compound of formula (II-2) is a compound of formula

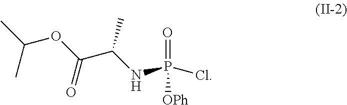 [SMILES: CC(C)OC(=O)[C@H](C)N[P@@](=O)(Cl)Oc1ccccc1]

8′. The process of any one of embodiments 1′ to 7′, wherein the compound of formula (III) is a compound of formula

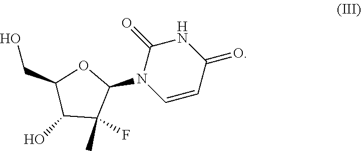 [SMILES: C[C@@]1(F)[C@H](O)[C@@H](CO)O[C@H]1n1ccc(=O)[nH]c1=O]

9′. The process of any one of embodiments 1′ to 7′, wherein R₇ and R₈ are independently H or methyl, wherein the compound of formula (III) is preferably

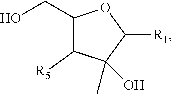

more preferably

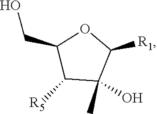

more preferably

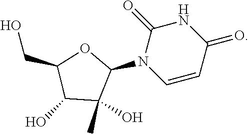 [SMILES: C[C@@]1(O)[C@H](O)[C@@H](CO)O[C@H]1n1ccc(=O)[nH]c1=O]

10′. The process of embodiment 9′, wherein the compound of formula (I-1) is a compound

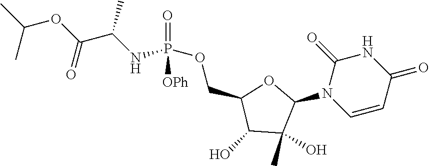 [SMILES: CC(C)OC(=O)[C@H](C)N[P@](=O)(OC[C@H]1O[C@@H](n2ccc(=O)[nH]c2=O)[C@](C)(O)[C@@H]1O)Oc1ccccc1]

and the compound of formula (I-2) is a compound

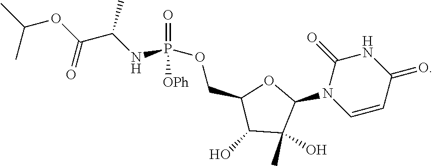 [SMILES: CC(C)OC(=O)[C@H](C)N[P@@](=O)(OC[C@H]1O[C@@H](n2ccc(=O)[nH]c2=O)[C@](C)(O)[C@@H]1O)Oc1ccccc1]

11′. A process for preparing of a compound of formula (I)

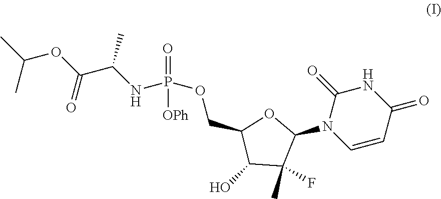 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a salt thereof, the process comprising
a′) reacting a compound of formula (II)

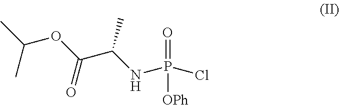 [SMILES: CC(C)OC(=O)[C@H](C)NP(=O)(Cl)Oc1ccccc1]

with a compound of formula (III)

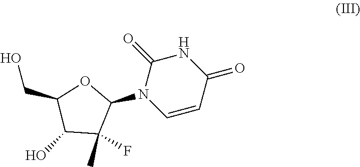 [SMILES: C[C@@]1(F)[C@H](O)[C@@H](CO)O[C@H]1n1ccc(=O)[nH]c1=O]

in the presence of a hydrogen chloride binding base which is not N-methylimidazole, obtaining a mixture comprising the compound of formula (I), wherein the compound of formula (II) comprises a compound of formula (II-1)

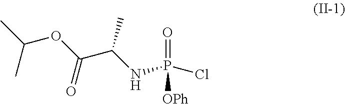 [SMILES: CC(C)OC(=O)[C@H](C)N[P@](=O)(Cl)Oc1ccccc1]

and a compound of formula (II-2)

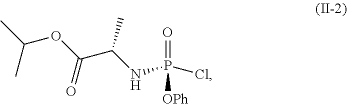 [SMILES: CC(C)OC(=O)[C@H](C)N[P@](=O)(Cl)Oc1ccccc1]

wherein the molar ratio of the compound of formula (II-1) relative the compound of formula (II-2) is preferably in the range of from 55:45 to 45:55, wherein the hydrogen chloride binding base is not N-methylimidazole.
12′. The process of embodiment 11′, wherein the molar ratio of the compound of formula (II-1) relative the compound of formula (II-2) is in the range of from 52:48 to 48:52, more preferably in the range of from 51:49 to 49:51.
13′. The process of embodiment 11′ or 12′, wherein the molar ratio of the compound of formula (II-1) relative the compound of formula (II-2) is 1:1.
14′. The process of any one of embodiments 11′ to 13′, wherein the compound of formula (II) consists of the compound of formula (II-1) and the compound of formula (II-2).
15′. The process of any one of embodiments 11′ to 14′, wherein in the mixture obtained from a′), the compound of formula (I) comprises a compound of formula (I-1)

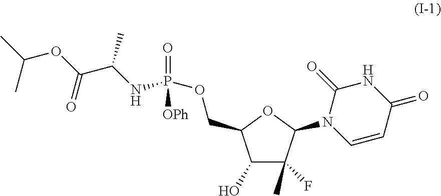 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and a compound of formula (I-2)

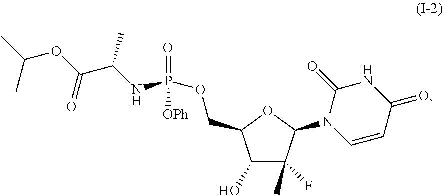 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

wherein the molar ratio of the compound of formula (I-1) relative to the compound of formula (I-2) is at least 65:35.
16′. The process of embodiment 15′, wherein the molar ratio of the compound of formula (I-1) relative to the compound of formula (I-2) is at least 70:30, preferably at least 75:25.
17′. The process of embodiment 15′ or 16′, wherein the molar ratio of the compound of formula (I-1) relative to the compound of formula (I-2) is at least 80:20.
18′. The process of any one of embodiments 11′ to 17′, wherein in the mixture obtained from a′), the compound of formula (I) consists of a compound of formula (I-1) and a compound of formula (I-2).
19′. The process of any one of embodiments 1′ to 18′, preferably 11′ to 18′, wherein the hydrogen chloride binding base is an organic base, preferably an organic nitrogenous base.
20′. The process of embodiment 19′, wherein the organic hydrogen chloride binding base comprises one or more of an amine, an amidine, and a heteroaromatic compound comprising a basic ring-nitrogen atom.
21′. The process of embodiment 19′ or 20′, wherein the organic hydrogen chloride binding base comprises one or more of ethyldiisopropylamine, triethylamine, diethylamine, 1,8-diazabicycloundec-7-ene, pyridine, quinoline, isoquinoline, acridine, pyrazine, imidazole, benzimidazole, and pyrazole.
22′. The process of any one of embodiments 1′ to 21′, preferably 11′ to 21′, wherein the hydrogen chloride binding base comprises, preferably is, triethylamine.
23′. The process of any one of embodiments 1′ to 22′, preferably 11′ to 22′, wherein prior to the reaction according to a′), the molar ratio of the hydrogen chloride binding base relative to the compound of formula (III) is in the range of from 0.1:1 to 5:1.
24′. The process of any one of embodiments 1′ to 23′, preferably 11′ to 23′, wherein prior to the reaction according to a′), the molar ratio of the hydrogen chloride binding base relative to the compound of formula (III) is in the range of from 0.5:1 to 5:1, preferably in the range of from 1:1 to 5:1, more preferably in the range of from 2:1 to 5:1.
25′. The process of any one of embodiments 1′ to 24′, preferably 11′ to 24′, wherein prior to the reaction according to a′), the molar ratio of the hydrogen chloride binding base relative to the compound of formula (III) is in the range of from 2:1 to 4:1, preferably in the range of from 2.5:1 to 4:1, more preferably in the range of from 2.5:1 to 3.5:1.
26′. The process of any one of embodiments 1′ to 25′, preferably 11′ to 25′, wherein according to a′), the compound of formula (II) is reacted with the compound of formula (III) in the presence of the hydrogen chloride binding base and in the presence of a Lewis acid.
27′. The process of embodiment 26′, wherein the Lewis acid comprises a twice positively charged ion or a three times positively charged ion.
28′. The process of embodiment 26′ or 27′, wherein the Lewis acid comprises a twice positively charged metal ion or a three times positively charged metal ion.
29′. The process of embodiment 27′ or 28′, wherein the twice positively charged ion is a Zn ion, a Mg ion, a Cu ion, or an Fe ion.
30′. The process of any one of embodiments 27′ to 29′, wherein the twice positively charged ion is a Zn ion.
31′. The process of any one of embodiments 26′ to 30′, wherein the Lewis acid comprises, preferably is, one or more of ZnBr₂, ZnCl₂, and ZnI₂.
32′. The process of any of one of embodiments 25′ to 29′, wherein the Lewis acid comprises, preferably is, ZnBr₂.
33′. The process of embodiments 26′, wherein the one or more Lewis acids is one or more of ZnBr₂, ZnCl₂, ZnI₂, MgBr₂, MgBr₂.OEt₂, CuCl₂, Cu(acetylacetonate)₂, and Fe(II) fumarate.
34′. The process of embodiment 27′ or 28′, wherein the three times positively charged ion is a Mn ion.
35′. The process of any one of embodiments 26′ to 28′ or 34′, wherein the Lewis acid comprises, preferably is, Mn(acetylacetonate)₃.
36′. The process of any one of embodiments 1′ to 35′, preferably 11′ to 35′, wherein prior to the reaction according to a′), the molar ratio of the Lewis acid relative to the compound of formula (III) is in the range of from 0.1:1 to 5:1.
37′. The process of any one of embodiments 1′ to 36′, preferably 11′ to 36′, wherein prior to the reaction according to a′), the molar ratio of the Lewis acid relative to the compound of formula (III) is in the range of from 0.2:1 to 5:1, preferably in the range of from 0.5:1 to 3:1, more preferably in the range of from 0.75:1 to 1.5:1.
38′. The process of any one of embodiments 1′ to 37′, preferably 11′ to 37′, wherein prior to the reaction according to a′), the molar ratio of the compound of formula (II) relative to the compound of formula (III) is in the range of from 0.5:1 to 5:1.
39′. The process of any one of embodiments 1′ to 38′, preferably 11′ to 38′, wherein the molar ratio of the compound of formula (II) relative to the compound of formula (III) is in the range of from 0.8:1 to 2:1, preferably in the range of from 0.9:1 to 1.2:1.
40′. The process of any one of embodiments 1′ to 39′, preferably 11′ to 39′, wherein according to a′), the compound of formula (II) is reacted with the compound of formula (III) in the presence of the hydrogen chloride binding base, preferably in the presence of a Lewis acid, and in the presence of a solvent.
41′. The process of embodiment 40′, wherein the solvent comprises, preferably is, one or more organic solvents.
42′. The process of embodiment 40′ or 41′, wherein the solvent comprises, preferably is, one or more aprotic organic solvents.
43′. The process of any one of embodiments 40′ to 42′, wherein the solvent comprises one or more of methylene chloride, methyl tert-butyl ether, tetrahydrofuran, dimethylsulphoxide, and dimethylformamide.
44′. The process of any one of embodiments 40′ to 43′, wherein the solvent comprises, preferably is, tetrahydrofuran.
45′. The process of any one of embodiments 1′ to 44′, preferably 11′ to 44′, wherein the reacting according to a′) is carried out at a temperature in the range of from 0 to 80° C., preferably in the range of from 0 to 25° C.
46′. The process of any one of embodiments 1′ to 45′, preferably 11′ to 45′, wherein the reacting according to a′) is carried out at a temperature the range of from 0 to 5° C.
47′. The process of any one of embodiments 1′ to 46′, preferably 11′ to 46′, wherein the reacting according to a′) is carried out for a period of time in the range of from 0.5 to 48 h, preferably in the range of from 1 to 36 h.
48′. The process of any one of embodiments 1′ to 47′, preferably 11′ to 47′, wherein the reacting according to a′) is carried out for a period of time in the range of from 2 to 24 h.
49′. The process of any one of embodiments 1′ to 48′, preferably 11′ to 48′, further comprising b′) separating the compound of formula (I) from the mixture obtained in a′).
50′. The process of embodiment 49′, wherein the separating according to b′) comprises filtration, centrifugation, drying, or a combination of two or more thereof.
51′. The process of embodiment 50′, wherein drying comprises drying in an atmosphere comprising oxygen, nitrogen, or a mixture thereof.
52′. The process of embodiment 50′ or 51′, wherein drying comprises rapid-drying, preferably spray-drying.
53′. The process of any one of embodiments 49′ to 52′, wherein the separating in b′) comprises separating the compound of formula (I) from the mixture obtained in a′), obtaining the compound of formula (I) dissolved in a solvent, preferably an organic solvent.
54′. The process of embodiment 53′, further comprising c′) crystallizing the compound of formula (I-1), preferably from the mixture obtained from b′) comprising the compound of formula (I) dissolved in a solvent.
55′. The process of embodiment 54′, wherein the crystallizing in c) comprises seeding, preferably seeding with seed crystals of the compound of formula (I-1).
56′. The process of embodiment 54′ or 55′, wherein the separating according to b′) comprises
   c′) crystallizing the compound of formula (I-1), preferably from the mixture obtained from b′) comprising the compound of formula (I) dissolved in a solvent, obtaining the crystallized compound of formula (I-1) in its mother liquor;
   d′) separating the crystallized compound of formula (I-1) from its mother liquor, obtaining the compound of formula (I-1) in crystalline form, said separating comprising
      d1′) subjecting the mother liquor comprising the crystallized compound of formula (I-1) to filtration, obtaining a filter cake comprising the compound of formula (I-1);
      d2′) optionally washing the filter cake comprising the compound of formula (I-1);
      d3′) drying the optionally washed filter cake, obtaining the compound of formula (I-1).
57′. The process according to any one of embodiments 1′ to 56′, wherein step a′) is carried out in a solvent wherein the solvent is not an anhydrous solvents selected from dichloromethane, 2-methyl tetrahydrofuran, tetrahydrofuran, methyl-t-butyl ether, ethyl acetate, acetonitrile, cyclopentyl methylether, 1,4-dioxane, acetone, methyl ethyl ketone, methyl isobutyl ketone, diisopropyl ethyl amine, tripropylamine, tributylamine and their combinations, and any functional equivalent thereof.
58′. The process according to any one of embodiments 1′ to 57′, wherein the base is not selected from tripropyl amine, tributyl amine, diisopropyl ethyl amine, and their combinations, and any functional equivalent thereof.
59′. The process according to any one of embodiments 1 to 58′, wherein the base selected from tripropylamine, tributylamine, diisopropylethylamine or any functional equivalent bases is not in combination with a solvent selected from dichloromethane, 2-methyl tetrahydrofuran, tetrahydrofuran, methyl-t-butyl ether, ethyl acetate, acetonitrile, cyclopentyl methylether, 1,4-dioxane, acetone, methyl ethyl ketone, methyl isobutyl ketone, diisopropyl ethyl amine, tripropylamine.
60′. The process according to any one of embodiments 1′ to 59′, wherein when the base is tripropylamine the solvent is not acetone or methyl-isobutyl-ketone or methyl-t-butyl ether or ethyl acetate or when the base is diisopropylethylamine the solvent is not methyl-t-butyl ether or when the base is THF the solvent is not tributylamine.
61′. A mixture comprising a compound of formula (I), obtainable or obtained from step a′) of a process according to any one of embodiments 1′ to 59′, preferably 11′ to 56′.
62′. A mixture, preferably a mixture of embodiment 61′, comprising the compound of formula (I)

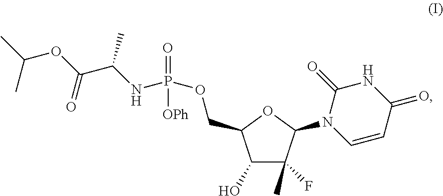 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

wherein the compound of formula (I) comprises a compound of formula (I-1)

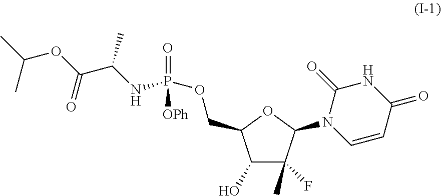 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and a compound of formula (I-2)

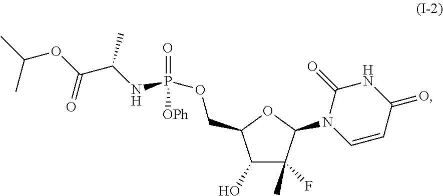 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

wherein in the mixture, the molar ratio of the compound of formula (I-1) relative to the compound of formula (I-2) is at least 65:35, preferably at least 70:30, more preferably at least 75:25, more preferably at least 80:20, said mixture further comprising a hydrogen chloride binding base, which is not N-methylimidazole, with bound hydrogen chloride.
63′ The mixture of embodiment 62′, wherein the hydrogen chloride binding base is an organic hydrogen chloride binding base, preferably comprising one or more of an amine, an amidine, and a heteroaromatic compound comprising a basic ring-nitrogen atom, more preferably comprising one or more of ethyldiisopropylamine, triethylamine, diethylamine, 1,8-diazabicycloundec-7-ene, pyridine, quinoline, isoquinoline, acridine, pyrazine, imidazole, benzimidazole, and pyrazole, the hydrogen chloride binding base more preferably comprising, more preferably being, triethylamine.
64′. The mixture of embodiment 62′ or 63′, further comprising a Lewis acid preferably comprising a twice positively charged ion or a three times positively charged ion, more preferably a twice positively charged metal ion or a three times positively charged metal ion, more preferably a twice positively charged metal ion preferably being a Zn ion, a Mg ion, a Cu ion, or an Fe ion, preferably a Zn ion, the Lewis acid more preferably comprising, more preferably being, one or more of ZnBr₂, ZnCl₂, and ZnI₂, more preferably comprising, more preferably being, ZnBr₂.
65′. The mixture of any one of embodiments 62′ to 64′, further comprising a solvent preferably comprising one or more organic solvents, more preferably one or more aprotic organic solvents, more preferably comprising one or more of methylene chloride, methyl tert-butyl ether, tetrahydrofuran, dimethylsulphoxide, and dimethylformamide, wherein more preferably, the solvent comprises, more preferably is, tetrahydrofuran.
66′. Use of a mixture of any one of embodiments 62′ to 65′ for obtaining the compound of formula (I-1)

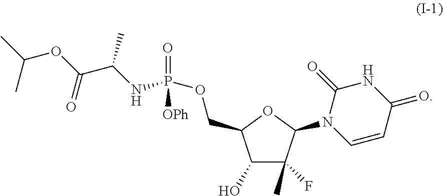 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

67′. The use of embodiment 66′, wherein obtaining the compound of formula (I-1) comprises separating the compound of formula (I) from the mixture and crystallizing the compound of formula (I-1), obtaining the crystallized compound of formula (I) in its mother liquor and separating the crystallized compound of formula (I-1) from its mother liquor.
68′. A mixture comprising a compound of formula (II)

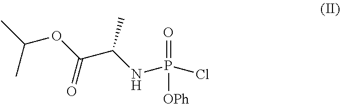 [SMILES: CC(C)OC(=O)[C@H](C)NP(=O)(Cl)Oc1ccccc1]

and a compound of formula (III)

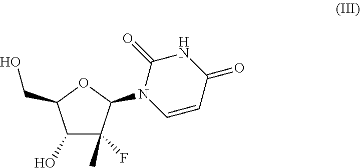 [SMILES: C[C@@]1(F)[C@H](O)[C@@H](CO)O[C@H]1n1ccc(=O)[nH]c1=O]

and a hydrogen chloride binding base which is not N-methylimidazole, wherein the compound of formula (II) comprises a compound of formula (II-1)

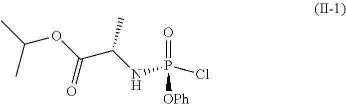 [SMILES: CC(C)OC(=O)[C@H](C)N[P@](=O)(Cl)Oc1ccccc1]

and a compound of formula (II-2)

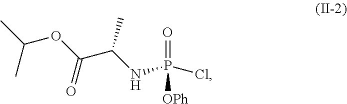 [SMILES: CC(C)OC(=O)[C@H](C)N[P@](=O)(Cl)Oc1ccccc1]

wherein the molar ratio of the compound of formula (II-1) relative the compound of formula (II-2) is preferably in the range of from 55:45 to 45:55, more preferably in the range of from 52:48 to 48:52, more preferably in the range of from 51:49 to 49:51, wherein more preferably, the molar ratio of the compound of formula (II-1) relative the compound of formula (II-2) is 1:1.
69′. The mixture of embodiment 68′, wherein the hydrogen chloride binding base is an organic hydrogen chloride binding base, preferably comprising one or more of an amine, an amidine, and a heteroaromatic compound comprising a basic ring-nitrogen atom, more preferably comprising one or more of ethyldiisopropylamine, triethylamine, diethylamine, 1,8-diazabicycloundec-7-ene, pyridine, quinoline, isoquinoline, acridine, pyrazine, imidazole, benzimidazole, and pyrazole, the hydrogen chloride binding base more preferably comprising, more preferably being, triethyl amine.
70′. The mixture of embodiment 68′ or 69′, wherein the molar ratio of the hydrogen chloride binding base relative to the compound of formula (III) is in the range of from 0.1:1 to 5:1, preferably in the range of from 0.5:1 to 5:1, more preferably in the range of from 1:1 to 5:1, more preferably in the range of from 2:1 to 5:1, more preferably in the range of from 2:1 to 4:1, more preferably in the range of from 2.5:1 to 4:1, more preferably in the range of from 2.5:1 to 3.5:1.
71′. The process of any one of embodiments 68′ to 69′, wherein the molar ratio of the compound of formula (II) relative to the compound of formula (III) is in the range of from 0.5:1 to 5:1, preferably in the range of from 0.8:1 to 2:1, more preferably in the range of from 0.9:1 to 1.2:1.
72′. The mixture of any one of embodiments 68′ or 71′, further comprising a Lewis acid preferably comprising a twice positively charged ion or a three times positively charged ion, more preferably a twice positively charged metal ion or a three times positively charged metal ion, more preferably a twice positively charged metal ion preferably being a Zn ion, a Mg ion, a Cu ion, or an Fe ion, preferably a Zn ion, the Lewis acid more preferably comprising, more preferably being, one or more of ZnBr₂, ZnCl₂, and ZnI₂, more preferably comprising, more preferably being, ZnBr₂.
73′. The mixture of embodiment 68, wherein the molar ratio of the Lewis acid relative to the compound of formula (III) is in the range of from 0.1:1 to 5:1, preferably in the range of from 0.2:1 to 5:1, more preferably in the range of from 0.5:1 to 3:1, more preferably in the range of from 0.75 to 1.5:1.
74′. The mixture of any one of embodiments 68′ to 73′, further comprising a solvent preferably comprising one or more organic solvents, more preferably one or more aprotic organic solvents, more preferably comprising one or more of methylene chloride, methyl tert-butyl ether, tetrahydrofuran, dimethylsulphoxide, and dimethylformamide, wherein more preferably, the solvent comprises, more preferably is, tetrahydrofuran.
75′. Use of a mixture according to any one of embodiments 68′ to 74′ for preparing a compound of formula (I-1)

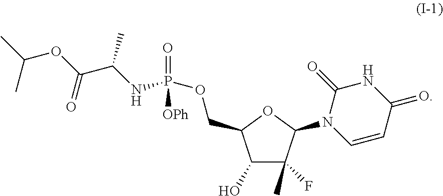 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

76′. Use of a mixture according to any one of embodiments 68′ to 75′ for improving the selectivity to the compound of formula (I-1)

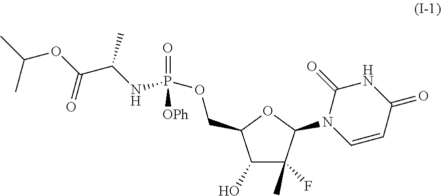 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

of the reaction of a compound of a compound of formula (III) with a compound of formula (II) comprising a compound of formula (II-1) and a compound of formula (II-2) wherein the molar ratio of the compound of formula (II-1) relative the compound of formula (II-2) is preferably in the range of from 55:45 to 45:55, more preferably in the range of from 52:48 to 48:52, more preferably in the range of from 51:49 to 49:51, wherein more preferably, the molar ratio of the compound of formula (II-1) relative the compound of formula (II-2) is 1:1.
77′. Use of a combination of a Lewis acid and a hydrogen chloride binding base which is not N-methylimidazole for improving the selectivity to the compound of formula (I-1)

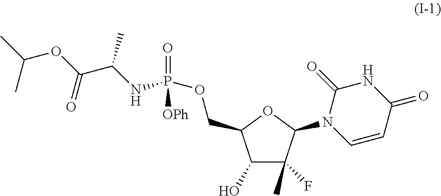 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

of the reaction of a compound of a compound of formula (III)

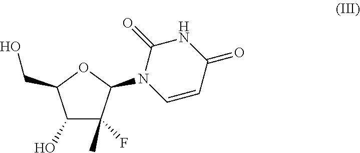 [SMILES: C[C@@]1(F)[C@H](O)[C@@H](CO)O[C@H]1n1ccc(=O)[nH]c1=O]

with a compound of formula (II)

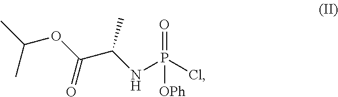 [SMILES: CC(C)OC(=O)[C@H](C)NP(=O)(Cl)Oc1ccccc1]

said compound of formula (II) comprising a compound of formula (II-1)

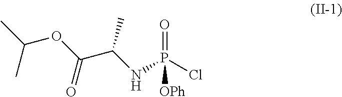 [SMILES: CC(C)OC(=O)[C@H](C)N[P@](=O)(Cl)Oc1ccccc1]

and a compound of formula (II-2)

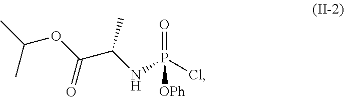 [SMILES: CC(C)OC(=O)[C@H](C)N[P@](=O)(Cl)Oc1ccccc1]

wherein the molar ratio of the compound of formula (II-1) relative the compound of formula (II-2) is preferably in the range of from 55:45 to 45:55, more preferably in the range of from 52:48 to 48:52, more preferably in the range of from 51:49 to 49:51, wherein more preferably, the molar ratio of the compound of formula (II-1) relative the compound of formula (II-2) is 1:1.
78′. The use of embodiment 77′, wherein the hydrogen chloride binding base is an organic hydrogen chloride binding base, preferably comprising one or more of an amine, an amidine, and a heteroaromatic compound comprising a basic ring-nitrogen atom, more preferably comprising one or more of ethyldiisopropylamine, triethylamine, diethylamine, 1,8-diazabicycloundec-7-ene, pyridine, quinoline, isoquinoline, acridine, pyrazine, imidazole, benzimidazole, and pyrazole, the hydrogen chloride binding base more preferably comprising, more preferably being, triethyl amine.
79′. The use of embodiment 77′ or 78′, wherein the Lewis acid comprises a twice positively charged ion or a three times positively charged ion, more preferably a twice positively charged metal ion or a three times positively charged metal ion, more preferably a twice positively charged metal ion preferably being a Zn ion, a Mg ion, a Cu ion, or an Fe ion, preferably a Zn ion, the Lewis acid more preferably comprising, more preferably being, one or more of ZnBr₂, ZnCl₂, and ZnI₂, more preferably comprising, more preferably being, ZnBr₂.
80′. A compound of formula (I)

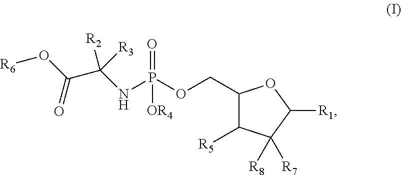

preferably a compound of formula (I)

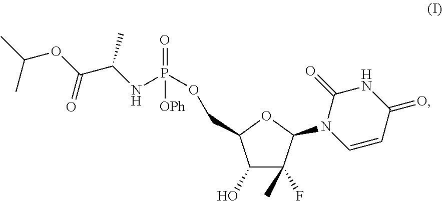 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

more preferably a compound of formula (I-1)

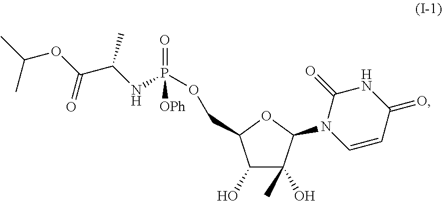 [SMILES: CC(C)OC(=O)[C@H](C)N[P@](=O)(OC[C@H]1O[C@@H](n2ccc(=O)[nH]c2=O)[C@](C)(O)[C@@H]1O)Oc1ccccc1]

obtainable or obtained by a process according to any one of embodiments 1 to 56, preferably according to any one of embodiments 49′ to 62′, more preferably according to embodiment 56′.
81′. Use of the compound of embodiment 80′ for the preparation of a pharmaceutical composition.
82′. A method of using the compound of embodiment 80′ for the preparation of a pharmaceutical composition.
83′. A pharmaceutical composition, comprising the compound of embodiment 80′ and preferably at least one pharmaceutically acceptable excipient.
84′. The pharmaceutical composition of embodiments 83′ for use in a method for treating hepatitis C in a human.
85′. Use of the pharmaceutical composition of embodiments 83′ or 84′ for treating hepatitis C in a human.
86′. A method of treating hepatitis C in a human comprising administering the pharmaceutical composition of embodiments 83′ or 84′ to a human.
87′. Use of the crystalline form of sofosbuvir according to any of embodiments 1′ to 7′, or the crystalline form of sofosbuvir obtainable or obtained by a process according to any of embodiments 8′ to 26′ for preparing a medicament for the treatment hepatitis C in a human.
88′. Use of the compound of embodiment 80′ for the treatment of hepatitis C in a human.
89′. The compound of embodiment 80′ for use in the treatment of hepatitis C in a human.
90′. The compound of embodiment 80′ for the treatment of hepatitis C in a human.
91′. A method of treating hepatitis C in a human comprising administering the compound of embodiment 80′ to a human.

The present invention is further illustrated by the following examples, comparative examples, and references examples.

### EXAMPLES

### List of Abbreviations

DCM dichloromethane
dr (or. d.r.) diastereomeric excess
equiv equivalents
HPLC high pressure liquid chromatography
M molar, molarity
MTBE methyl tert-butyl ether
NMI N-methylimidazole
NMR nuclear magnetic resonance
TEA triethylamine
THF tetrahydrofuran
   General Analytical Methods Reactions were monitored by HPLC on a C-18 reverse phase column with a gradient of acetonitrile in 10 mM ammonium sulfamate aqueous buffer at pH 5.6. NMR spectra were recorded in CDCl₃ or DMSO on a 300 MHz spectrometer. Diastereomeric excess of sofosbuvir (3) was determined using the same HPLC method. Diastereomeric excess of N-hydroxysuccinimide phosphoramidate (Sp)-2 was determined using ³¹P NMR. ¹H, and ¹³C chemical shifts are reported in ppm relative to TMS (0 ppm) with the solvent resonance as the internal standard (DMSO, ¹H: 2.50 ppm, ¹³C: 39.52 ppm, CDCl₃, ¹H: 7.26 ppm, ¹³C: 77.16 ppm). ³¹P NMR chemical shifts are reported in ppm relative to 85% phosphoric acid (0 ppm).

### Succinimide as Leaving Group

### Example 1: Coupling with Non-Diastereopure II-a—Demonstration of Diastereoselectivity

### Example 1.1 Synthesis of the Compound of Formula (II-a) with N-Hydroxysuccinimide as LG and Crystallization Thereof to Obtain Compound of Formula (II-a′) (dr 67:33)

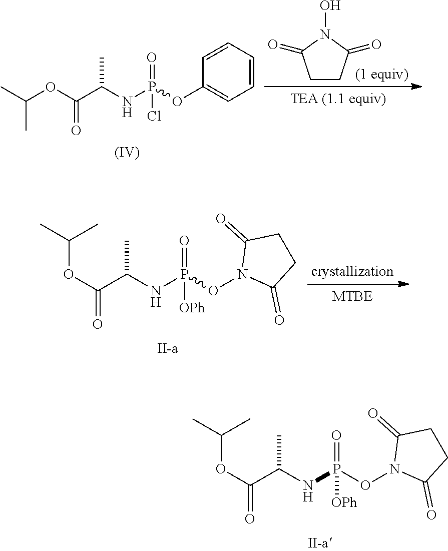

In a dry two-neck round bottom flask equipped with a dropping funnel was dissolved crude phosphoryl chloride (IV) prepared according J. Org. Chem 2011, 76, 8311 (20 g, 43.8 mmol, 1 equiv, 67% w/w purity by NMR) in dichloromethane (140 mL) and the solution was cooled to ca. 5° C. with an ice bath. N-hydroxysuccinimide (7.53 g, 65.4 mmol, 1 equiv) was added (only partial dissolution).

To this suspension, triethylamine (10 mL, 71.9 mmol, 1.1 equiv) in dichloromethane (20 mL) was added dropwise with stirring, and the dropping funnel was rinsed with a further 5 mL of dichloromethane, whereby all of N-hydroxysuccinimide dissolved and a precipitation of triethylamine hydrochloride was observed. The ice bath was removed, the reaction mixture was allowed to warm up to room temperature and extracted with 90 mL of distilled water. The organic phase was washed with a further 40 mL of distilled water and the volatiles were removed under reduced pressure. The crude solid was dissolved in 160 mL MTBE (methyl tert-butyl ether), charged with 5 mL triethylamine and left to stir overnight, upon which a solid agglomerate was formed. The mixture was diluted with 75 mL of MTBE and warmed up to 50° C. until all of the solid dissolved. Upon cooling, crystals formed which were filtered and dried to give 10.53 g of II-a′(dr 67:33).

### Example 1.2

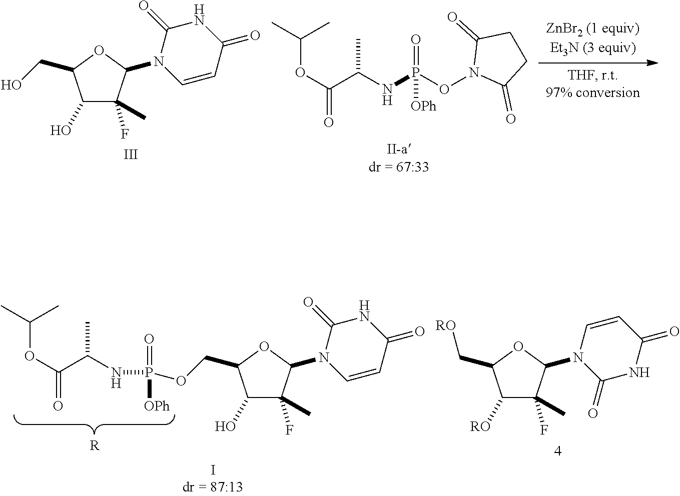

To a two-neck round bottom flask equipped with a reflux condenser and purged with nitrogen was added N-hydroxysuccinimide phosphoramidate II-a′ with dr=76:33 (Sp:Rp) (463 mg, 1.20 mmol, 1.56 equiv) prepared according to Example 1.1 and THF (2.5 mL, anhydrous) to obtain a clear solution.

To this solution was added 2′-deoxy-2′-fluoro-2′C-methyluridine III (200 mg, 0.77 mmol, 1 equiv), followed by ZnBr₂ (173 mg, 0.77 mmol, 1 equiv), 4 Å molecular sieves (235 mg) and Et₃N (320 mL, 2.31 mmol, 3 equiv). The mixture was stirred at room temperature for 4 hours. HPLC analysis with individual response factor correction indicated 3% of unreacted nucleoside III, 94% of sofosbuvir (I) with dr=87:13 (Sp:Rp), and 2% of 3′,5′-bis-phosphoramidate impurity 4 (dr not determined).

### Example 1.3

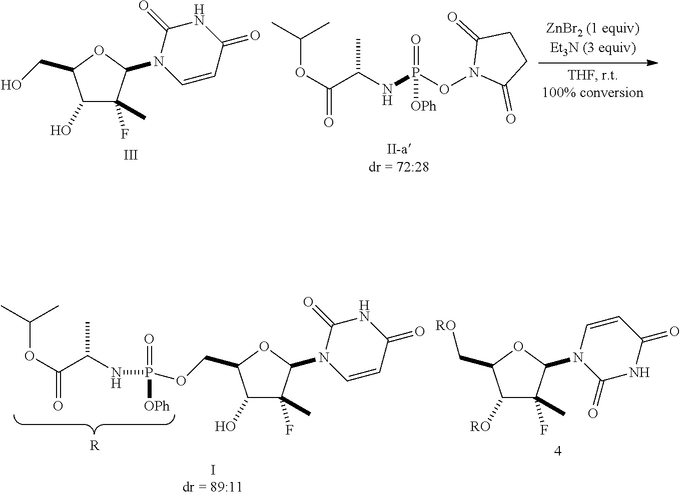

To a two-neck round bottom flask equipped with a reflux condenser and purged with nitrogen was added N-hydroxysuccinimide phosphoramidate II-a′ with dr=72:28 (Sp:Rp) (493 mg, 1.28 mmol, 1.67 equiv) prepared according to Example 1.1 (further crystallized in MTBE to obtain (Sp)-2 with a dr 72:28) and THF (2.5 mL, anhydrous) to obtain a clear solution.

To this solution was added 2′-deoxy-2′-fluoro-2′C-methyluridine III (200 mg, 0.77 mmol, 1 equiv), followed by ZnBr₂ (173 mg, 0.77 mmol, 1 equiv), 4 Å molecular sieves (235 mg) and Et₃N (320 mL, 2.31 mmol, 3 equiv). The mixture was stirred at room temperature for 4 hours. HPLC analysis with individual response factor correction indicated complete conversion of III, 95% of sofosbuvir (I) with dr=89:11 (Sp:Rp), and 5% of 3′,5′-bis-phosphoramidate impurity 4 (dr not determined).

### Example 2: Coupling with P-Racemic II-0

### Example 2.1: Preparation of P-Racemic Phosphoramidate II-0

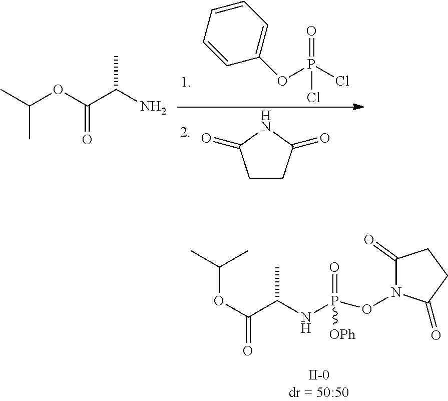

In a jacketed reactor equipped with a mechanical stirrer, a nitrogen bubbler and a dropping funnel was dissolved L-alanine isopropyl ester (20.0 g, 119.3 mmol, 1 equiv) in THF (200 mL) and the internal temperature set to 0° C. To this solution, phenyl phosphorodichloridate (18.8 mL, 95% purity, 119.5 mmol, 1 equiv) was added at 20° C., followed by a dropwise addition of triethylamine (34.8 mL, 250 mmol, 2.1 equiv) over 2 h at 0-7° C., upon which a white precipitate was formed. The solution was stirred at 0° C. for 2 h 20 min. N-hydroxysuccinimide (17.8 g, 154.7 mmol, 1.3 equiv) was added, followed by additional triethylamine (24.8 mL, 177.9 mmol, 1.5 equiv) which was added dropwise over 56 min. The reaction was stirred at 0° C. for 17.5 h, diluted with MTBE (1200 mL), stirred at 0° C. for 1 h and filtered over a Nutsche filter twice. The filtrate was concentrated to 100 mL, diluted with MTBE to a total volume of 600 mL and filtered over a plug of silica (20 g). The resulting clear filtrate (650 mL) was cooled to 20° C. The solution was cooled gradually to −5° C., stirred for 2 h at this temperature and filtered over a Nutsche filter. The solid was dried at 40° C. under vacuum to afford 38.7 g II-0 (100.7 mmol, 84%, dr=49.8:50.2 as determined by ³¹P NMR in DMSO-d6:5.3 ppm (Rp-diastereomer), 4.3 (Sp-diastereomer)).

### Example 2.2: Synthesis and Re-Crystallization

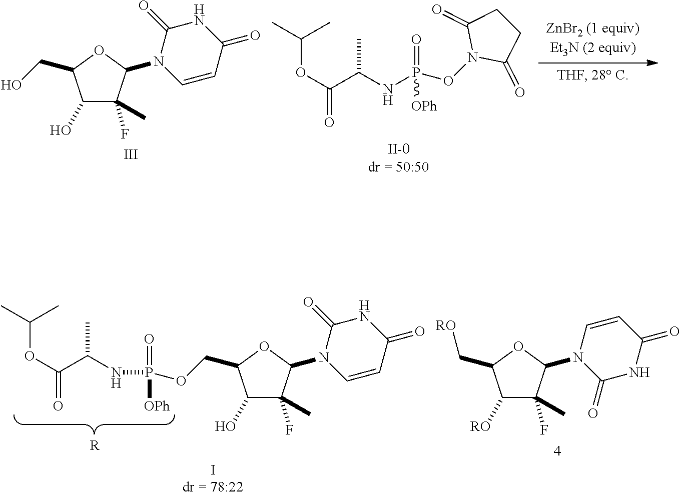

In a three-neck round bottom flask equipped with nitrogen bubbler, N-hydroxysuccinimide phosphoramidate II-0, prepared according to Example 2.1 (10 g, 25.7 mmol, 1.2 equiv, dr=50:50 (Sp:Rp)) was dissolved in THF (125 mL). To this solution were added 4 A mol. sieves (6.7 g), 2′-deoxy-2′-fluoro-2′C-methyluridine 1 (5.58 g, 21.4 mmol, 1 equiv), ZnBr₂ (4.82 g, 21.4 mmol, 1 equiv) and triethylamine (5.93 mL, 42.8 mmol, 2 equiv). The resulting thick suspension was stirred at 28° C. for 16.5 h. HPLC analysis with individual response factor correction indicated 2% of III, 96% of sofosbuvir (I) with dr=78:22 (Sp:Rp), and 1% of 3′,5′-bis-phosphoramidate impurity 4 (dr not determined). The reaction was filtered over a Nutsche filter, washing with THF (10 mL) and charged with 1M HCl (100 mL). This solution was distilled at 45° C./80 mbar until no more distillate was observed to obtain a 2-phase water/oil mixture. This mixture was charged with dichloromethane (95 mL) and the phases were separated. The dichloromethane phase was concentrated to an end mass of 68 g, and transferred to a jacketed reaction vessel with a mechanical stirrer, pre-warmed to 35° C. The solution gradually cooled to 20° C., seeded with crystals of sofosbuvir at this temperature and stirred for 17 h at 15° C., 1 h at 0° C. and 2 h at −10° C. The crystal suspension was filtered over a Nutsche filter washing with chilled dichloromethane (2×5 mL) and dried under vacuum at 40° C. The product I (sofosbuvir) was obtained as white crystals (HPLC purity 99%, dr=97:3, 5.17 g, 9.76 mmol, 46%). Of this product, 4.5 g were dissolved in THF (45 mL) and charged with 1M HCl (40 mL). This solution was distilled at 40° C./80 mbar until an end mass of 37.8 g. This 2-phase mixture was charged with dichloromethane (36 mL) and the phases were separated. The dichloromethane phase was concentrated to an end mass of 23 g, warmed to 35° C., whereby it crystallized spontaneously, and cooled to 0° C. over 2 h. The crystal suspension was stirred for 1 h at 0° C. and 15.5 h at −10° C. The crystal suspension was filtered over a Nutsche filter washing with chilled dichloromethane (2×1 mL) and dried under vacuum at 40° C. The product I (sofosbuvir) was obtained as white crystals (HPLC purity >99%, dr=99.4:0.6, 4.01 g, 89%).

### Example 2.3: Table Entry 8

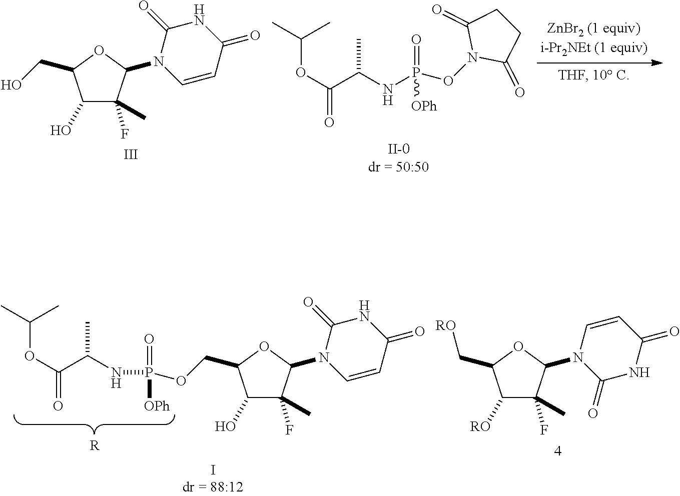

In a two-neck round bottom flask equipped with nitrogen bubbler, N-hydroxysuccinimide phosphoramidate II-0, prepared according to Example 2.1 (468 mg, 1.2 mmol, 5 equiv, dr=50:50 (Sp:Rp)) was dissolved in THF (1.5 mL). To this solution were added 4 A mol. sieves (152 mg), 2′-deoxy-2′-fluoro-2′C-methyluridine III (63.3 mg, 0.24 mmol, 1 equiv) and ZnBr₂ (59 mg, 0.26 mmol, 1.1 equiv). The suspension was cooled to 10° C. and Hünig's base (82 mL, 0.24 mmol, 1 equiv) was added. The reaction mixture was stirred at 10° C. for 24 h. HPLC analysis with individual response factor correction indicated complete consumption of III, 97% of sofosbuvir (I) with dr=88:12 (Sp:Rp), and 3% of 3′,5′-bis-phosphoramidate impurity 4 (dr not determined).

The following syntheses were carried out following the procedure of example 2.2 (entry 8 of the table) with the conditions varied as specified in Table 1 below.

TABLE 1

| | Equiv | | Base | ZnBr₂ | | | | % | dr |
|---|---|---|---|---|---|---|---|---|---|
| Entry | II-0 | Base | equiv | equiv | Temp | Time | | SOFOS | SOFOS |
| 1 | 3 | TEA | 2 | 1 | r.t. | 15.5 | h | 96% | 81:19 |
| 2 | 5.1 | TEA | 3 | 2 | r.t. | 4 | h | 96% | 73:27 |
| 3 | 5.1 | TEA | 2 | 0.5 | r.t. | 4 | h | 92% | 82:18 |
| 4 | 5.1 | TEA | 2 | 0.25 | r.t. | 4 | h | 94% | 75:25 |
| 5 | 2 | TEA | 1.5 | 1 | r.t. | 20.5 | h | 96% | 83:17 |
| 6 | 5.1 | DBU | 1 | 1 | r.t. | 4 | h | 95% | 87:13 |
| 7 | 5 | PIP | 1 | 1 | 10° C. | 4 | h | 47% | 79:21 |
| 8 | 5 | Hünig | 1 | 1 | 10° C. | 24 | h | 97% | 88:12 |
| 9 | 5 | TMG | 1 | 1 | 10° C. | 4 | h | 95% | 88:12 |
| 10 | 2.2 | DBU | 1 | 1 | r.t. | 3 | h | 72% | 86:14 |
| 11 | 3 | EPH | 2 | 1 | r.t. | 4 | h | 72% | 86:14 |
| 12 | 3 | Hünig | 3 | 1 | 10° C. | 21 | h | 92% | 89:11 |

TEA = triethylamine
PIP = piperidine
TMG = tetramethylguanidine
EPH = ephedrine,
DBU = diazabicycloundecen,
Hünig = ethyldiisopropylamine

The reactions were monitored via 1H NMR. The 1H NMR experiments showed that the dr of the phosphorylating agent II-0 remains constant at 50:50, while sofosbuvir is formed with a diastereoisomeric excess as indicated in the dr SOFOS column of above table 1. This indicates that the dynamic resolution of III is occurring.

### Example 2.4: Coupling with Dr 1:1 of Compound 2 in DMF as Solvent

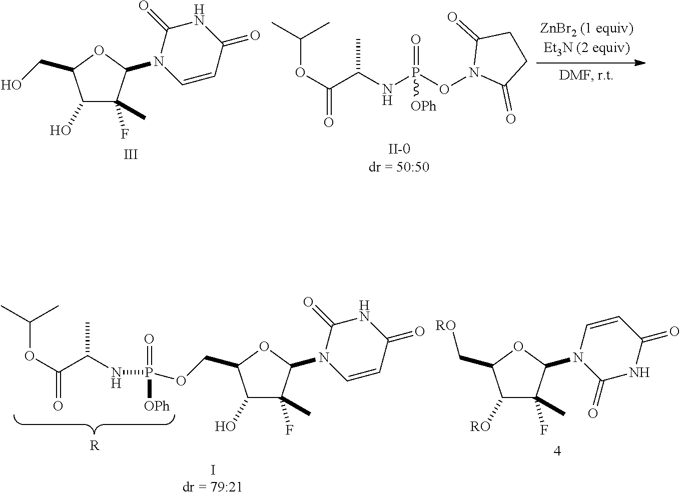

To a two-neck round bottom flask equipped with a reflux condenser and purged with nitrogen was added N-hydroxysuccinimide phosphoramidate II-0 with dr=50:50 (Sp:Rp) (177 mg, 0.46 mmol, 1.2 equiv) prepared according to example 2.1 and DMF (2.88 mL) to obtain a clear solution. To this solution was added 4 Å molecular sieves (67 mg), 2′-deoxy-2′-fluoro-2′C-methyluridine III (100 mg, 0.38 mmol, 1 equiv), ZnBr₂ (86 mg, 0.38 mmol, 1 equiv), and Et₃N (106 mL, 0.77 mmol, 2 equiv). The mixture was stirred at room temperature for 18.5 hours. HPLC analysis with individual response factor correction indicated 60% of unreacted nucleoside III, 39% of sofosbuvir (I) with dr=79:21 (Sp:Rp), and 1% of 3′,5′-bis-phosphoramidate impurity 4 (dr not determined).

### Comparative Example 2.1: Chloro-Phosphate Coupling with NMI

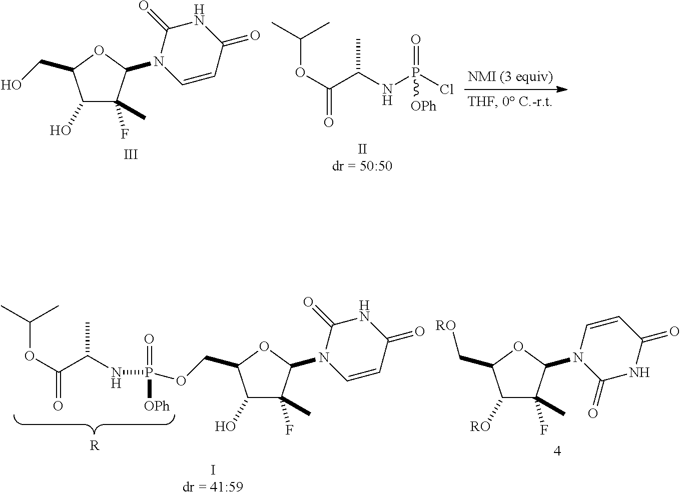

To a two-neck round bottom flask equipped with a reflux condenser and purged with nitrogen was added 2′-deoxy-2′-fluoro-2′C-methyluridine III (100 mg, 0.38 mmol, 1 equiv) followed by a THF solution of chlorophosphate II (dr=1:1, 1.15 mmol, 3 equiv in 3.52 mL THF). The slightly turbid solution was cooled to 0° C., charged with NMI (196 mL, 2.46 mmol, 6.4 equiv) and stirred at 0° C. for 15 min and at room temperature for 2.5 h. HPLC analysis with individual response factor correction indicated 78% of III, 9% of sofosbuvir (I) with dr=41:59 (Sp:Rp), and 13% of 3′,5′-bis-phosphoramidate impurity 4 (dr not determined).

### Comparative Example 2.2: Coupling with tBuMgCl

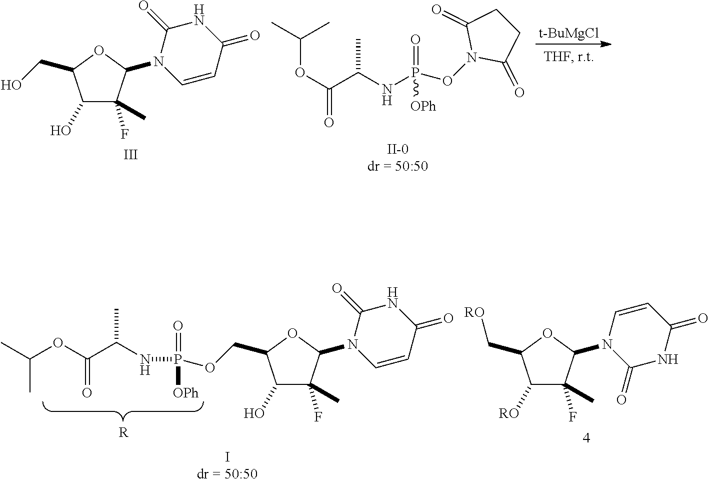

To a two-neck round bottom flask equipped with a reflux condenser and purged with nitrogen was added 2′-deoxy-2′-fluoro-2′C-methyluridine III (100 mg, 0.384 mmol, 1 equiv) followed by a THF (4 mL). The suspension was heated until most of the material dissolved and cooled to room temperature. To this solution were added 4 Å molecular sieves (130 mg) followed by N-hydroxysuccinimide phosphoramidate II-0 (dr=1:1, 221 mg, 0.58 mmol, 1.5 equiv). To this suspension was added t-BuMgCl (1M in THF, 422 mL, 0.42 mmol, 1.1 equiv), and the reaction was stirred at room temperature. After 18 h, HPLC analysis with individual response factor correction indicated 32% of III, 36% of sofosbuvir (I) with dr=61:39 (Sp:Rp), and 33% of 3′,5′-bis-phosphoramidate impurity 4 (dr not determined).

### Comparative Example 2.3: Coupling with tBuMgCl and ZnBr₂

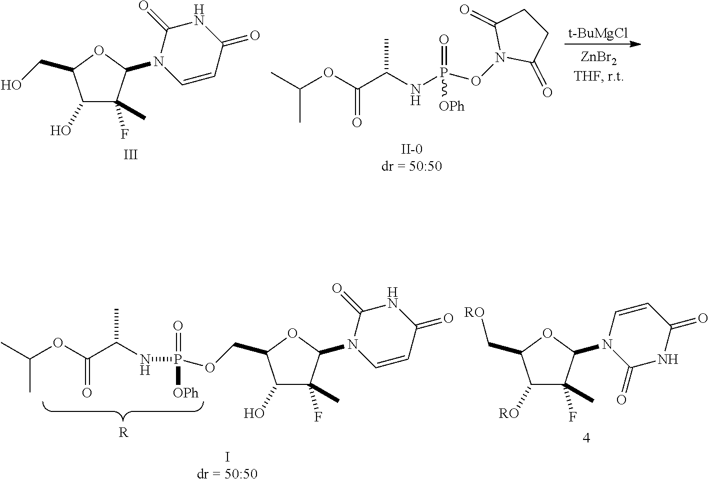

To a two-neck round bottom flask equipped with a reflux condenser and purged with nitrogen was added 2′-deoxy-2′-fluoro-2′C-methyluridine III (1 g, 3.84 mmol, 1 equiv) followed by a THF (5 mL). The suspension was heated until most of the material dissolved and cooled to room temperature. To this solution was added consecutively t-BuMgCl (1M in THF, 7.98 mL, 7.98 mmol, 1.5 equiv), ZnBr₂ (898 mg, 3.99 mmol, 1.05 equiv) [caution: exotherm!] and a solution N-hydroxysuccinimide phosphoramidate II-0 (dr=1:1, 2.07 g, 5.39 mmol, 1.42 equiv) in THF (5 mL). The reaction was stirred at room temperature. After 18 h, HPLC analysis with individual response factor correction indicated 65% of III, 5% of sofosbuvir (I) with dr=47:53 (Sp:Rp), and 29% of 3′,5′-bis-phosphoramidate impurity 4 (dr not determined).

### Results

As shown in Comparative Example 2.1, the coupling of the chloro-phosphoramidate in the presence of the NMI, as taught in the art, leads to a compound (I) with a dr of 41:59. Compared to Comparative Example 1, Examples 1.1, 1.2 and 2.2 and 2.3 the dr ratio of the compound I is quite higher in favor of the valuable stereoisomer. It was very surprising that when using the phosphoramidate derivative according to the invention in combination with a base, in particular the Hünig base and trimethylamine and with a Lewis acid better dr can be obtained compared to the use of a chloro-phosphoramidate in the presence of NMI. The use of a higher excess of compound (II) relative to compound (III) further improve the diastereoselectivity of the process. Furthermore, as shown in Comparative Example 2.2 and 2.3, the use of non-nitrogenous base (t-BuMgCl) with or without a Lewis acid leads to much lower yield and diastereoselectivity for the valuable stereoisomer of I.

### Example 3: Synthesis of the Compound of Sofosbuvir as Compound of Formula (I-1) by Via Chlorophosphate (Compound of Formula (II))

### Example 3.1: Chlorophosphate Coupling with ZnBr₂ and TEA—Improved Conditions (Lower Excess of Compound II)

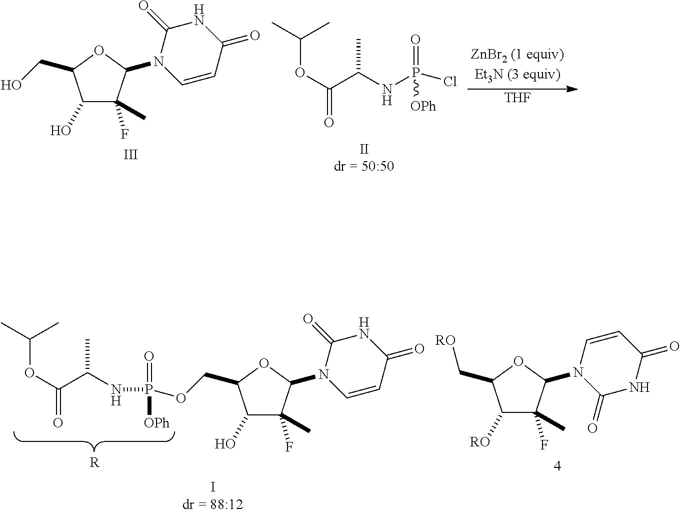

To a two-neck round bottom flask equipped with a reflux condenser and purged with nitrogen was added 20 wt % THF solution of chlorophosphate II (dr=1:1, 874 mg, 0.58 mmol, 1.5 equiv), followed by 2′-deoxy-2′-fluoro-2′C-methyluridine III (100 mg, 0.38 mmol, 1 equiv) and THF (1.8 mL). To this solution was added ZnBr₂ (86 mg, 0.38 mmol, 1 equiv) and the suspension was cooled to 0° C. Triethylamine (160 mL, 1.15 mmol, 3 equiv) was added and the mixture was stirred at 0° C. for 10 min and allowed to warm up to room temperature. After 18.5 h, HPLC analysis with individual response factor correction indicated 4% of III, 89% of sofosbuvir (I) with dr=89:11 (Sp:Rp), and 7% of 3′,5′-bis-phosphoramidate impurity 4 (dr not determined).

### Example 3.2: Chloro-Phosphate Coupling with ZnBr₂ and TEA, with MIBK as Solvent

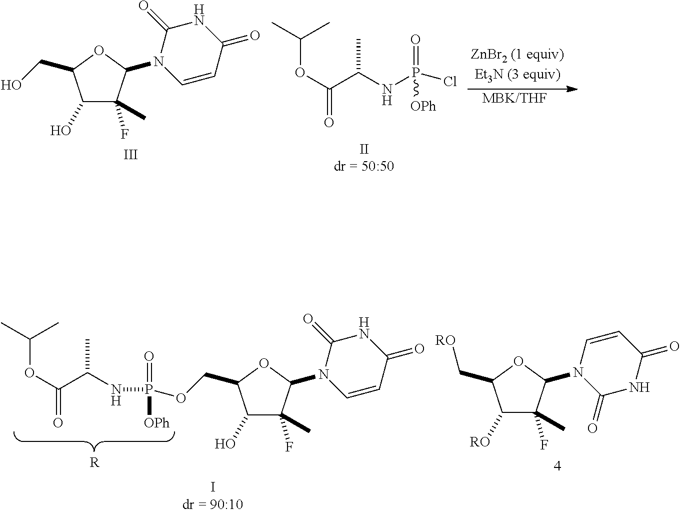

To a two-neck round bottom flask equipped with a reflux condenser and purged with nitrogen was added 20 wt % THF solution of chlorophosphate II (dr=1:1, 874 mg, 0.58 mmol, 1.5 equiv), followed by 2′-deoxy-2′-fluoro-2′C-methyluridine III (100 mg, 0.38 mmol, 1 equiv) and MIBK (1.8 mL). To this solution was added ZnBr₂ (86 mg, 0.38 mmol, 1 equiv) and the suspension was cooled to 0° C. Triethylamine (160 mL, 1.15 mmol, 3 equiv) was added and the mixture was stirred at 0° C. for 10 min and allowed to warm up to room temperature. After 2 h, HPLC analysis with individual response factor correction indicated 38% of III, 58% of sofosbuvir (I) with dr=91:9 (Sp:Rp), and 4% of 3′,5′-bis-phosphoramidate impurity 4 (dr not determined). After 18 h, HPLC analysis with individual response factor correction indicated 33% of III, 62% of sofosbuvir (I) with dr=90:10 (Sp:Rp), and 5% of 3′,5′-bis-phosphoramidate impurity 4 (dr not determined).

### Example 4: Coupling in the Presence of a Hydrogen Chloride Binding Base (Trimethylamine)

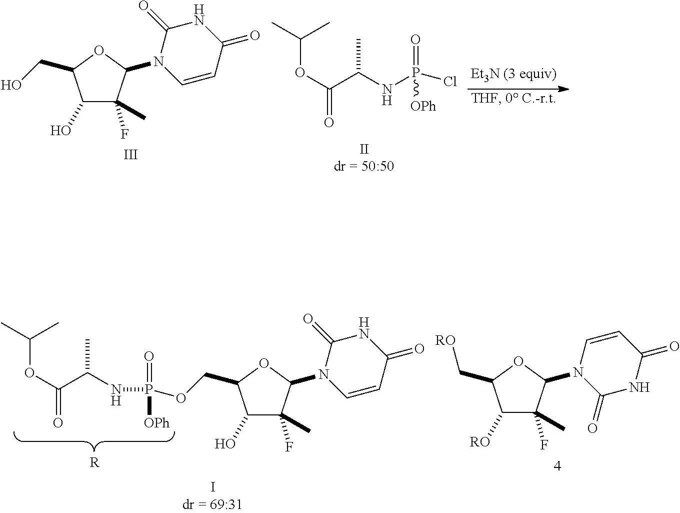

To a two-neck round bottom flask equipped with a reflux condenser and purged with nitrogen was added 2′-deoxy-2′-fluoro-2′C-methyluridine III (100 mg, 0.38 mmol, 1 equiv) followed by a THF solution of chlorophosphate II (dr=1:1, 0.96 mmol, 2.5 equiv in 2.94 mL THF). The slightly turbid solution was cooled to 0° C., charged with triethylamine (160 microL, 1.15 mmol, 3 equiv) and stirred at 0° C. for 2.5 h and at room temperature for 14.5 h. HPLC analysis with individual response factor correction indicated complete conversion of III, 97% of sofosbuvir (I) with dr=69:31 (Sp:Rp), and 3% of 3′,5′-bis-phosphoramidate impurity 4 (dr not determined).

### Comparative Example 3.1: Coupling in the Presence of a Lewis Acid (ZnBr₂) and N-Methylimidazole

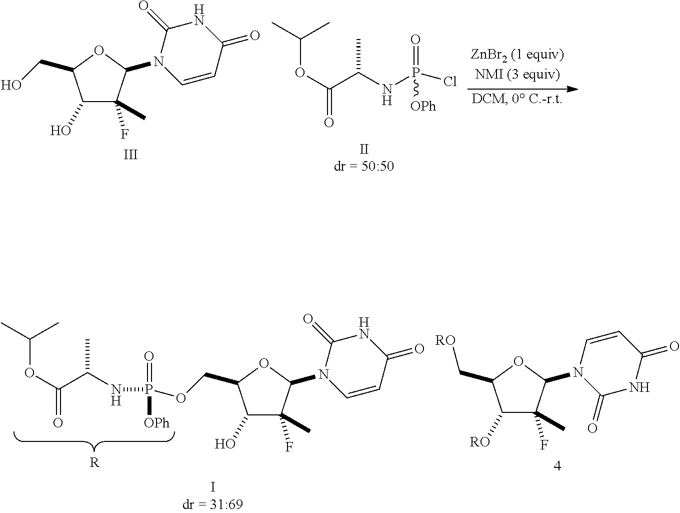

To a two-neck round bottom flask equipped with a reflux condenser and purged with nitrogen was added 2′-deoxy-2′-fluoro-2′C-methyluridine III (100 mg, 0.38 mmol, 1 equiv) followed by a DCM solution of chlorophosphate II (dr=1:1, 0.96 mmol, 2.5 equiv in 2.94 mL DCM). The solution was charged with ZnBr₂ (86 mg, 0.38 mmol, 1 equiv), cooled to 0° C. and charged with NMI (92 microL, 1.15 mmol, 3 equiv). The resulting suspension and stirred at 0° C. for 2.5 h and at room temperature for 14.5 h. HPLC analysis with individual response factor correction indicated 66% of III, 18% of sofosbuvir (I) with dr=31:69 (Sp:Rp), and 16% of 3′,5′-bis-phosphoramidate impurity 4 (dr not determined).

### Comparative Example 3.2: Chloro-Phosphate Coupling with tBuMgCl

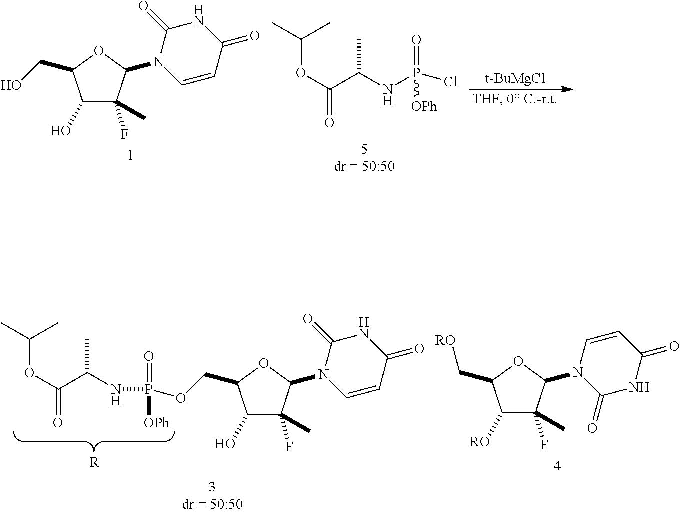

To a two-neck round bottom flask equipped with a reflux condenser and purged with nitrogen was added 2′-deoxy-2′-fluoro-2′C-methyluridine 1 (100 mg, 0.38 mmol, 1 equiv) followed by a THF (4 mL). The suspension was heated until clear and cooled to room temperature. To this solution were added 4 Å molecular sieves (130 mg) and a 20 wt % solution of chlorophosphate 5 (dr=1:1, 874 mg, 0.58 mmol, 1.5 equiv). To this suspension was added t-BuMgCl (1M in THF, 422 mL, 0.42 mmol, 1.1 equiv), and the reaction was stirred at room temperature. After 17.5 h, HPLC analysis with individual response factor correction indicated 28% of 1, 13% of sofosbuvir (3) with dr=50:50 (Sp:Rp), and 59% of 3′,5′-bis-phosphoramidate impurity 4 (dr not determined).

### Results

Example 4 where a hydrogen chloride binding base which is not NMI and which is, in particular, trimethylamine, shows a very good result, illustrated by the dr of 69:31. If in addition to NMI a Lewis acid is employed (see Comparative Example 2.1), the result is even worse than when using NMI alone, illustrated by the dr of 31:69. Therefore, it was very surprising that when using a hydrogen chloride binding base which is not NMI and which is, in particular, trimethylamine, in combination with a Lewis acid (see Example 3.1 to 3.2), an even better dr can be obtained (80:20) compared to the use a hydrogen chloride binding base which is not NMI alone.

### CITED PRIOR ART

WO 2008/121634 A
WO 2011/123668 A
WO 2010/135569 A
WO 2011/123645 A
WO 2011/123672 A
WO 2014/047117 A
WO 2014/164533 A
J. Org. Chem. 2011, 76, 8311

1. A process for preparing of a compound of formula (I) 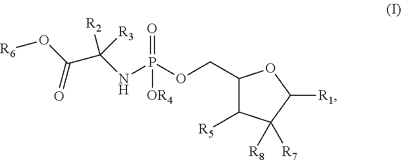
or a salt thereof, the process comprising
a) reacting a compound of formula (II) 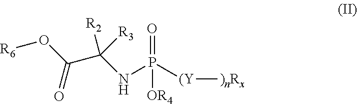
wherein (Y—)ₙRₓ is a leaving group for nucleophilic substitution reaction, wherein n is 0 or 1 and wherein Y is O, N or S, with a compound of formula (III) 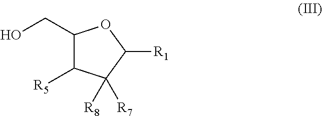
in the presence of a base and a Lewis acid and obtaining a mixture comprising the compound of formula (I) and
wherein
R₄ is phenyl, naphthyl, quinolinyl, isoquinolinyl, quinazolinyl or quinoxalinyl, each optionally substituted with at least one of C₁-C₆ alkyl, C₁-C₆ alkoxy, C₃-C₆ cycloalkyl, aryl, halogen, C(O)OH, CHO, C(O)(C₁-C₆ alkyl), C(O)(aryl), C(O)O(C₁-C₆ alkyl) C(O)ONH₂, C(O)ONH(C₁-C₆ alkyl) and CN;
R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, NO₂, C(O)OH, CHO, C(O)(C₁-C₆ alkyl), C(O)(aryl), C(O)O(C₁-C₆ alkyl), C(O)ONH₂, C(O)ONH(C₁-C₆ alkyl) and CN;
R₆ is C₁-C₆ alkyl or C₃-C₁₀ cycloalkyl optionally substituted with at least one of C₁-C₆ alkyl and aryl;
R₁ is an optionally derivatized purinyl residue, including an adenine residue and a guanine residue, or an optionally derivatized pyrimidinyl residue, including a cytosine residue, a thymine residue and an uracil residue, linked to the furanose ring according to formula (III) through a carbon or nitrogen atom;
R₇ and R₈ are independently H, OH, F, Cl, Br, I, azide, nitrile, NH₂, NHR₂₆, NR₂₆R₂₄, C(O)NH₂, C(O)NHR₂₆, C(O)NR₂₆R₂₄, C₁-C₆ alkyl optionally substituted with C₁-C₆ alkyl, or C₃-C₁₀ cycloalkyl optionally substituted with C₁-C₆ alkyl, wherein R₂₆ and R₂₄ are independently C₁-C₆ alkyl;
R₅ is H, OH, C₁-C₆ alkoxy, OC(O)R₂₅, or C₁-C₆ alkyl optionally substituted with C₁-C₆ alkyl or aryl, wherein R₂₅ is C₁-C₆ alkyl or aryl and wherein
when n is 1,
Rₓ is alkyl, aryl, or heteroaryl, each optionally substituted with one or more electron-withdrawing groups, or
Rₓ is a residue of formula (A) 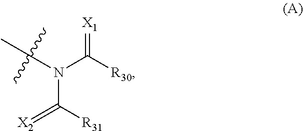
a residue of formula (B) 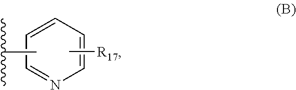
a residue of formula (C) 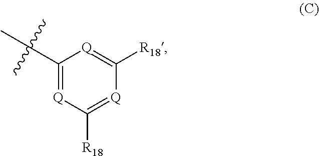
or a residue of formula (D) 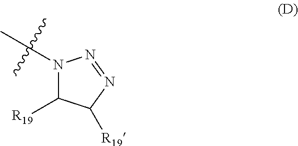
and wherein, when n is 0,
Rₓ is a residue of formula (A1) 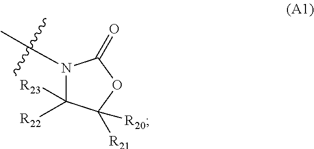
wherein at each occurrence
X₁ and X₂ are independently O or S;
R₃₀ and R₃₁ are independently H, OH, NH₂, C₁-C₆ alkyl or C₁-C₆ alkoxy, or
R₃₀ and R₃₁, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group:
R₁₈ and R_{18′} are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, or a heterocycle comprising one or more heteroatoms independently being N, O or S, the 5- or 6-membered optionally substituted ring;
R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, or a heterocycle comprising one or more heteroatoms independently being N, O or S, the 5-, 6-, or 7-membered saturated or partially unsaturated or aromatic ring.

2. (canceled)

3. The process of claim 1, wherein, when n is 1, Rₓ is selected form a residue of formula (A), a residue of formula (B), a residue of formula (C), a residue of formula (D), or when n is 0, Rₓ is selected form a residue of formula (A1).

4. The process of claim 1, wherein the base is an organic base.

5-9. (canceled)

10. The process of claim 1, wherein the Lewis acid comprises a twice positively charged ion or a three times positively charged ion.

11. The process of claim 1, wherein the Lewis acid comprises a twice positively charged metal ion or a three times positively charged metal ion.

12. The process of claim 10, wherein the twice positively charged ion is a Zn ion, a Mg ion, a Cu ion, or an Fe ion.

13. The process of claim 1, wherein the twice positively charged ion is a Zn ion.

14. The process of claim 1, wherein the Lewis acid comprises, one or more of ZnBr₂, ZnCl₂, and ZnI₂.

15. The process of claim 1, wherein the Lewis acid comprises ZnBr₂.

16. The process of claim 1, wherein the Lewis acids is one or more of ZnBr₂, ZnCl₂, ZnI₂, MgBr₂, MgBr₂.OEt₂, CuCl₂, Cu(acetylacetonate)₂, and Fe(II) fumarate, Mn(acetylacetonate)₃.

17-22. (canceled)

23. The process of claim 1, wherein n is 0 and Rₓ is Cl and wherein the base is not N-methylimidazole.

24. The process of claim 1, wherein n is 0 and Rₓ is Cl and wherein the base is not tert-butylmagnesium chloride.

25. The process of claim 1, wherein the compound of formula (II) comprises a compound of formula (II-A), 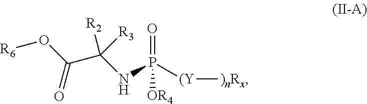
and a compound of formula (II-B), 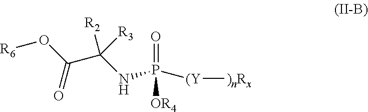
wherein the molar ratio of the compound of formula (II-A) relative the compound of formula (II-B) is in the range of from 45:55 to 72:28.

26. A mixture comprising a compound of formula (I), obtainable or obtained by a process according to claim 1.

27. A mixture comprising the compound of formula (I) 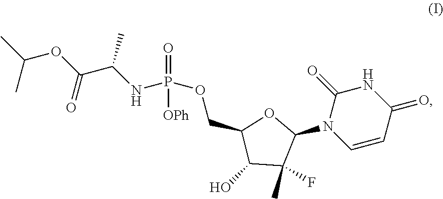 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]
wherein the compound of formula (I) comprises a compound of formula (I-1) 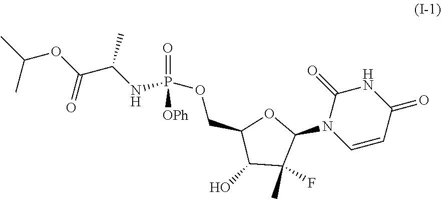 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]
and a compound of formula (I-2) 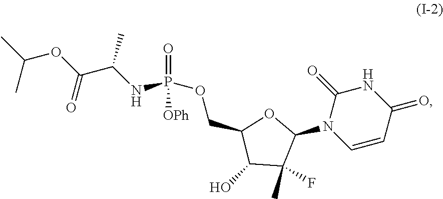 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]
wherein the molar ratio of the compound of formula (I-1) relative to the compound of formula (I-2) is at least 65:35.

28-32. (canceled)

33. A mixture comprising a compound of formula (II-0) 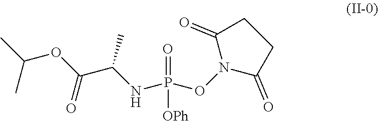 [SMILES: CC(C)OC(=O)[C@H](C)NP(=O)(Oc1ccccc1)ON1C(=O)CCC1=O]
and a compound of formula (III) 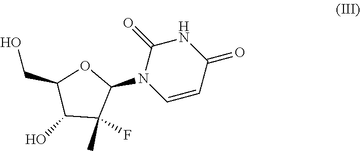 [SMILES: C[C@@]1(F)[C@H](O)[C@@H](CO)O[C@H]1n1ccc(=O)[nH]c1=O]
a base, and Lewis acid, wherein the compound of formula (II-0) comprises a compound of formula (II-a) 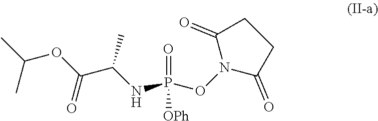 [SMILES: CC(C)OC(=O)[C@H](C)N[P@](=O)(Oc1ccccc1)ON1C(=O)CCC1=O]
and a compound of formula (II-b) 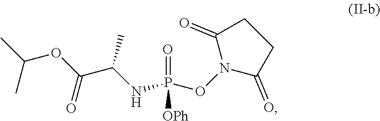 [SMILES: CC(C)OC(=O)[C@H](C)N[P@@](=O)(Oc1ccccc1)ON1C(=O)CCC1=O]
wherein the molar ratio of the compound of formula (II-a) relative the compound of formula (II-b) is in the range of from 45:55 to 72:28.

34-43. (canceled)

44. A process for preparing of a compound of formula (I) 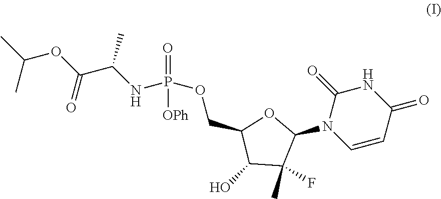 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]
or a salt thereof, the process comprising
a′) reacting a compound of formula (II) 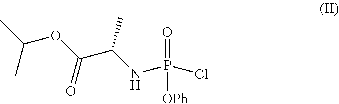 [SMILES: CC(C)OC(=O)[C@H](C)NP(=O)(Cl)Oc1ccccc1]
with a compound of formula (III) 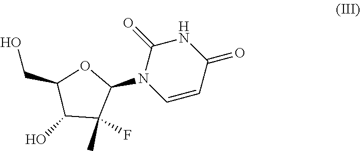 [SMILES: C[C@@]1(F)[C@H](O)[C@@H](CO)O[C@H]1n1ccc(=O)[nH]c1=O]
in the presence of a hydrogen chloride binding base which is not N-methylimidazole, obtaining a mixture comprising the compound of formula (I), wherein the compound of formula (II) comprises a compound of formula (II-1) 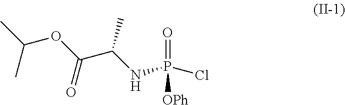 [SMILES: CC(C)OC(=O)[C@H](C)N[P@](=O)(Cl)Oc1ccccc1]
and a compound of formula (II-2) 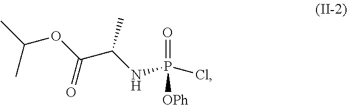 [SMILES: CC(C)OC(=O)[C@H](C)N[P@](=O)(Cl)Oc1ccccc1]
wherein the molar ratio of the compound of formula (II-1) relative the compound of formula (II-2) is in the range of from 55:45 to 45:55, wherein the hydrogen chloride binding base is not N-methylimidazole.

45. (canceled)

46. The process of claim 44, wherein in the mixture obtained from a′), the compound of formula (I) comprises a compound of formula (I-1) 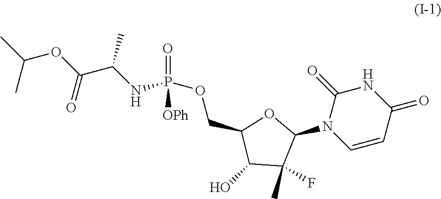 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]
and a compound of formula (I-2) 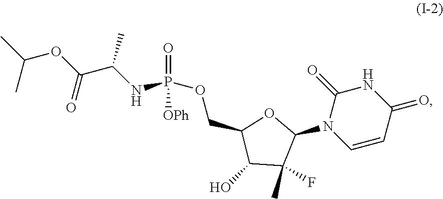 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]
wherein the molar ratio of the compound of formula (I-1) relative to the compound of formula (I-2) is at least 65:35.

47-58. (canceled)
